SEQUENCE LISTING (8 sequences)

SEQ ID NO: 1 - DNA, 28 bp, Homo sapiens
tggggagggtggggagggtggggaaggt

SEQ ID NO: 2 - DNA, 22 bp, Homo sapiens
tgagggtgggtagggtgggtaa

SEQ ID NO: 3 - DNA, 32 bp, Homo sapiens
agggcggtgtgggaagagggaagagggggagg

SEQ ID NO: 4 - DNA, 26 bp, Homo sapiens
ttagggttagggttagggttagggtt

SEQ ID NO: 5 - DNA, 19 bp, Artificial Sequence
  synthesized
gctgcttagacgctggatt

SEQ ID NO: 6 - DNA, 19 bp, Artificial Sequence
  synthesized
tcctcctcgtcgcagtaga

SEQ ID NO: 7 - DNA, 23 bp, Artificial Sequence
  synthesized
catgagaagtatgacaacagcct

SEQ ID NO: 8 - DNA, 22 bp, Artificial Sequence
  synthesized
agtccttccacgataccaaagt

(19) United States
(12) Patent Application Publication
(10) Pub. No.: US 2021/0382058 A1
(43) Pub. Date: Dec. 9, 2021

(54) PATIENT SELECTION FOR TREATMENT OF MYC POSITIVE CANCERS WITH INDENOISOQUINOLINES

(71) Applicant: Purdue Research Foundation, West Lafayette, IN (US)

(72) Inventors: Mark S. Cushman, West Lafayette, IN (US); Danzhou Yang, WEST LAFAYETTE, IN (US); Guanhui Wu, WEST LAFAYETTE, IN (US); Kaibo Wang, WEST LAFAYETTE, IN (US)

(21) Appl. No.: 17/333,384

(22) Filed: May 28, 2021

Related U.S. Application Data
(60) Provisional application No. 63/032,860, filed on Jun. 1, 2020.

(51) Int. Cl. G01N 33/574; C07D 471/04; C07D 217/16; C07D 401/06; C07D 491/056; G01N 33/542

(52) U.S. Cl. CPC G01N 33/57484; C07D 471/04; G01N 33/542; C07D 401/06; C07D 491/056; C07D 217/16

(57) ABSTRACT

The present disclosure is directed to a method for selecting a patient with cancer for treatment with a compound of formula (I) by determining if the patient's cancer cells are MYC-positive and when the MYC promoter sequence in those cancer cells contains a nucleic acid sequence capable of forming a MYC G-quadruplex (MYC G4) (i.e. are MYC G4-positive) and treating the patient with a compound of formula (I).

### CROSS-REFERENCE TO RELATED APPLICATIONS

This application claims the benefit under 35 U.S.C § 119(e) of U.S. Provisional Application No. 63/032,860 filed on Jun. 1, 2020, the entirety of the disclosure of which is incorporated herein by reference.

### GOVERNMENT SUPPORT CLAUSE

This invention was made with government support under contracts CA177585 and CA023168, both awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Jun. 4, 2021, is named seq_list_ST25_corrected.txt and is 1,605 bytes in size.

### TECHNICAL FIELD

The present disclosure generally relates to a method for selecting a patient with cancer for treatment and treating a patient with cancer, in particular to a method for selecting and treating a patient with cancer by using compounds that block the activities of MYC oncogene through binding the MYC promoter G-quadruplex. In some other embodiments, the invention disclosed herein relates to a method for treating a patient with cancer by a dual mechanism of action targeting both the MYC oncogene and human topoisomerase I.

### BACKGROUND

This section introduces aspects that may help facilitate a better understanding of the disclosure. Accordingly, these statements are to be read in this light and are not to be understood as admissions about what is or is not prior art.

Cancer is a group of most diverse diseases involving abnormal cell growth. Currently there are more than 100 types of identified cancer that affect human beings as well as animals. In 2016, there were an estimated 1,685,210 new human cancer cases diagnosed and 595,690 cancer deaths in the U.S. alone (Cancer Statistics 2016—American Cancer Society, Inc.). There are unmet and increasing needs for new and novel therapies for fighting cancers.

DNA is the target of many important anticancer agents, including human topoisomerase I inhibitors. Recently there has been significant progress in developing molecular-targeted therapies. A therapeutic advantage can be gained from DNA-targeted drugs combined with cancer-specific molecular targeting properties. Indenoisoquinolines are human topoisomerase I inhibitors with improved physicochemical and biological properties as compared to the traditional camptothecin topoisomerase I inhibitors that are clinically used for the treatment of various solid tumors.¹⁻⁶ Three indenoisoquinolines, indotecan (LMP400), indimitecan (LMP776), and LMP744 (FIG. 1A), have entered phase I clinical trials in adults with relapsed solid tumors and lymphomas.⁷⁻¹⁴ However, some indenoisoquinolines with potent anticancer activity surprisingly did not show strong topoisomerase I inhibition,^{3, 15} suggesting an additional mechanism of action. Notably, high concentrations of some indenoisoquinoline compounds have been reported to target DNA outside of topoisomerase I action.^{6-7, 16-17}

MYC is one of the most important oncogenes and is overexpressed in more than 80% of all types of cancer.¹⁸⁻¹⁹ The transcription factor MYC protein is involved in cell proliferation, differentiation, and apoptosis, and plays a pivotal role in tumor initiation and progression as well as drug resistance.²⁰′ MYC is found to be a general transcriptional “amplifier” in cancer cells.²⁵′ Even a brief inhibition of MYC expression has been shown to permanently stop tumor growth and induce tumor regression in vivo,²⁷ because of the “oncogene addiction” of tumor cells.²⁸ Therefore, MYC is a potential therapeutic target. However, the MYC protein is not an easy drug target due to its short half-life and lack of an apparent small molecule binding pocket.²⁹⁻³¹

The nuclease hypersensitive element (NHE) III₁ in the MYC promoter, which controls 85-90% of MYC transcriptional activity, forms a DNA G-quadruplex (G4) under transcription-associated negative supercoiling and functions as a transcriptional silencer (FIG. 1B, left).³²⁻³⁶ DNA G-quadruplexes (G4s) are globular four-stranded secondary structures consisting of stacked Hoogsteen hydrogen-bonded G-tetrads stabilized by K⁺ or Na⁺.³⁷ DNA G-quadruplexes found in promoter regions of key oncogenes have emerged as a promising new class of cancer-specific molecular targets for drug development.³⁸⁻⁴⁰ Using a G4-specific antibody, G4 structures have been visualized in human cells at both telomeric and non-telomeric sites on chromosomes, and G4-loci increase after exposure of live cells to G4 ligands.⁴¹ G4s detected in immortalized precancerous cells are at 10 times higher levels than in normal human cells, and G4-sites are found to be specifically enriched in regulatory, transcriptionally active regions of chromatin, particularly the MYC promoter region.⁴² The structures of the MYC promoter G-quadruplexes have been previously determined.⁴³⁻⁴⁴ The major MYC promoter G-quadruplex (MycG4) is a parallel-stranded structure with three G-tetrads connected by three propeller loops (FIG. 11B, right).^{32, 43, 45} Significantly, stabilization of the MYC promoter G-quadruplex by small molecules suppresses MYC transcription.^{32, 36, 46} For example, a quindoline anticancer agent was shown to stabilize the MYC G-quadruplex (abbreviated as MYC G4 or MycG4 interchangeably) and downregulate MYC.⁴⁶⁻⁴⁷ The molecular structure of the 2:1 quindoline-MycG4 complex has been determined, which shows specific recognition of the MycG4 by the crescent-shaped quindoline.⁴⁸ Indenoisoquinolines are crescent-shaped and share some structural similarity with the quindoline compound (FIG. 1(C)), which is consistent with the report that 6-substituted indenoisoquinolines¹⁵ bind the c-Kit promoter G4s.¹⁷

### BRIEF DESCRIPTION OF THE DRAWINGS

The above and other objects, features, and advantages of the present invention will become more apparent when taken in conjunction with the following description and drawings.

FIG. 1 Chemical structures of indenoisoquinoline topoisomerase I inhibitors in Phase I clinical trials and quindoline, as well as the MYC promoter and MYC promoter G-quadruplex. (A) Indenoisoquinoline topoisomerase I inhibitors currently in clinical trials. (B) Left: The structure of the human MYC gene promoter. The G4-forming region NHE III₁ sequence is shown, with the guanine runs underlined. The guanine runs involved in the formation of the major MycG4 are highlighted. Right: The folding topology of MycG4 adopted by the MycPu22 sequence is a parallel-stranded 3-tetrad G-quadruplex, with the two stabilizing potassium cations shown. (C) Left: a MycG4 stabilizer quindoline and a topoisomerase I inhibitor indenoisoquinoline. Right: overlay of the three-dimensional structures of quindoline and an indenoisoquinoline in their energy-minimized states.

FIG. 2 Indenoisoquinolines can induce and stabilize MycG4. (A) Left: schematic of the FRET-quenching assay used for compound screening. The FRET-quenching (shown as fluorophore in black color) caused by MycG4 folding can be induced by K⁺ or MycG4-inducing compounds. Right: relative fluorescence intensities of the labeled MycG4 in the presence of DMSO, 100 mM K⁺, and indenoisoquinoline analogs as shown by FRET-quenching assay. Data shown are the average values of two individual experiments. DMSO (negative control), 100 mM K⁺ (positive control), and six indenoisoquinolines used for further studies are highlighted and labeled. Conditions: 1 μM labeled DNA, 10 μM compound, 25° C., 50 mM Tris.acetate, pH 7. (B) Thermal stabilization values (ΔTₘ) of MycG4 by indenoisoquinoline analogs as shown by FRET-melting assay. Data shown are the average values of the two individual experiments. The six representative indenoisoquinolines used for further studies are highlighted and labeled. Conditions: 150 nM labeled DNA, 1.5 μM compound, 25° C., pH 7, 10 mM (C) Correlation of FRET-quenching and FRET-melting data. The Pearson correlation coefficient (r) is shown.

FIG. 3 MYC-inhibitory activities of indenoisoquinoline analogues. (A) MYC protein expression levels in the absence and presence of various concentrations of indenoisoquinolines (24 h treatment) were obtained by Western blotting experiments in MCF-7 breast cancer cells. GAPDH was used as an internal control. (B) Plot of the topoisomerase I inhibition levels against the MGM values of 31 indenoisoquinolines that were used to determine topoisomerase, MYC, and MGM activities. The enclosed region indicates more active indenoisoquinolines. Based on the MYC downregulation shown in the Western blotting results (panel A and FIGS. 12A to 12F), MYC inhibition levels were classified into four levels: strong inhibition, MYC expression inhibited at 0.5 to 1.0 μM; medium inhibition, MYC expression inhibited at 2.0 μM or no clear dose dependent MYC inhibition weak inhibition, MYC expression inhibited at 4.0 μM; no inhibition, no MYC expression inhibition up to 4.0 μM. The relative topoisomerase I (Top1) inhibition levels of the compounds were previously determined and classified into six levels (0-5).^{3, 6-9, 15, 50-52} The MGM values are the average of GI₅₀ values across the entire panel of NCI-60 cancer cell lines. The GI₅₀ values are the concentrations corresponding to 50% growth inhibition which were determined in the NCI-60 cancer cell lines drug screen (Table 3). (C) MYC transcription levels in the absence and presence of indenoisoquinolines (6 h treatment) were obtained by qRT-PCR experiments in MCF-7 cancer cells. DMSO was used as the negative control (no inhibition, 100%). The relative MYC mRNA levels were normalized with GAPDH. The experiments were run in triplicate. P values (***P<0.0004, ****P<0.0001) were determined by one-way ANOVA with post hoc Dunnett, relative to DMSO control.

FIG. 4 SAR of selected indenoisoquinolines. N.D., not determined.

FIG. 5A 1D ¹H NMR titrations of MycPu22 DNA with indenoisoquinolines and 7-azaindenoisoquinolines. Imino proton regions of the titration spectra of MycG4 with compound 5 (A), 6 (B), and 13 (C) are shown. In panel A (compound 5), the imino proton signals from the 5′ G-tetrad (FIG. 1B) are labeled 16, 7, 11, and 20, the imino proton signals from the middle G-tetrad are labeled 12, 21,17, and 8, and the imino proton signals from the 3′ G-tetrad are labeled 13, 22, 18, and 9. Conditions: 150 μM DNA, 25° C., pH 7, 100 mM K⁺.

FIG. 5B 1D ¹H NMR titrations of MycPu22 DNA with indenoisoquinolines and 7-azaindenoisoquinolines. Imino proton regions of the titration spectra of MycG4 with compound 9 (D), 12 (E), and 17 (F) are shown. Conditions: 150 μM DNA, 25° C., pH 7, 100 mM K+.

FIG. 6 A model of the 2:1 complex of 7-azaindenoisoquinoline 5:MycG4 suggested by Glide docking in different views. Intermolecular salt bridges are shown as black dashed lines.

FIG. 7 Binding selectivities of MYC G4-interactive indenoisoquinolines. Competition fluorescence displacement experiments with increasing concentrations of unlabeled G4s and dsDNA added to 3′-TAMRA-labeled MycPu22 (20 nM) mixed with 1 equivalent of compound 13 (A), 5 (B), 6 (C), 9 (D), and 12 (E). The normalized TAMRA fluorescence intensities at 580 nm were plotted as a function of molar ratio of added G4 DNA (in 3 G-tetrads) or calf thymus dsDNA (in 11 bp) to labeled MycPu22 DNA. The fluorescence intensity of free 3′-TAMRA labeled MycPu22 was defined as 100%, and 1:1 mixture of 3′-TAMRA labeled MycPu22 and indenoisoquinoline was defined as 0%. Conditions: 20° C., pH 7, 100 mM K⁺.

FIG. 8 (A) A schematic model showing the potential mechanisms of MYC suppression by indenoisoquinolines by (a) stabilization of MycG4 in the MYC promoter to inhibit transcription, and (b) inhibition of topoisomerase I to maintain negative supercoiling for G4 formation. (B) A heat map showing the synergistic effect of MYC inhibition and topoisomerase I inhibition on the anticancer activities of 29 indenoisoquinolines. The 29 indenoisoquinolines are grouped by their MYC inhibition levels and topoisomerase I inhibition levels. The anticancer activity for each group is determined by the mean(log₁₀ MGM) value of the grouped compounds (Table 4), which is displayed as gradient in the heat map. The MGM values are the approximate average of G150 values across the entire panel of NCI-60 cancer cell lines for each compound (Table 3). The synergistic effect of MYC inhibition and topoisomerase I inhibition is reflected by the increased anticancer activities (redder color) towards the bottom left corner with strong MYC and topoisomerase I inhibitory activities.

FIG. 9 Results from an MTS assay show dose-dependent cytotoxicity by active G4-interactive indenoisoquinolines in Raji cells (right bar) and CA46 cells (left bar). The Raji cells appear to be more sensitive than the CA46 cells at 100 nM and 300 nM treatments for 72 hours.

FIG. 10 Results of a 96-well in-cell western blot to examine the levels of MYC (top panel) and phosphorylated form of H2AX (γ-H2AX), a biomarker of DNA double-strand break (bottom panel), upon the treatment with the indicated indenoisoquinolines. The levels of target protein were normalized to cell number, in which cells were labeled with CellTag 700 staining.

FIG. 11 Fluorescence emission spectra of 5′-BHQ-MycPu28-3′-FAM (1 μM) or 5′-BHQ-MycPu22-3′-FAM (1 μM) in the presence or absence of 10 μM indenoisoquinoline 5. The levels of reduction in the fluorescence induced by indenoisoquinoline 5 are very similar for the MycPu28 and MycPu22, as shown by the numbers in parentheses. Conditions: 25° C., 50 mM Tris.acetate, pH 7.

FIG. 12A MYC protein expression levels in the absence and presence of various concentrations of the listed indenoisoquinolines (24 h treatment) obtained by western blotting experiments in MCF-7 breast cancer cell lines. GAPDH was used as an internal control.

FIG. 12B MYC protein expression levels in the absence and presence of various concentrations of listed indenoisoquinoline (24 h treatment) obtained by western blotting experiments in MCF-7 breast cancer cell lines. GAPDH was used as an internal control.

FIG. 12C MYC protein expression levels in the absence and presence of various concentrations of the listed indenoisoquinolines (24 h treatment) obtained by western blotting experiments in MCF-7 breast cancer cell lines. GAPDH was used as an internal control.

FIG. 12D MYC protein expression levels in the absence and presence of various concentrations of the listed indenoisoquinolines (24 h treatment) obtained by western blotting experiments in MCF-7 breast cancer cell lines. GAPDH was used as an internal control.

FIG. 12E MYC protein expression levels in the absence and presence of various concentrations of the listed indenoisoquinolines (24 h treatment) obtained by western blotting experiments in MCF-7 breast cancer cell lines. GAPDH was used as an internal control.

FIG. 12F MYC protein expression levels in the absence and presence of various concentrations of the listed indenoisoquinolines (24 h treatment) obtained by western blotting experiments in MCF-7 breast cancer cell lines. GAPDH was used as an internal control.

FIG. 13 Native PAGE experiments of MycPu22 G-quadruplex DNA in the presence and absence of various indenoisoquinolines. DNA bands were visualized using UV light. Each sample contains 4 μL of 150 μM DNA. Conditions: 25° C., TBE buffer containing 12.5 mM KCl, pH 8.0.

FIG. 14A CD spectra of MycPu22 G-quadruplex DNA (15 μM) with addition of 1, 2, 3, and 4 equivalents of indenoisoquinoline compound 9 (A), 5 (C), and 13 (E) Conditions: 25° C., pH 7, 5 mM K⁺.

FIG. 14B CD spectra of MycPu22 G-quadruplex DNA (15 μM) with addition of 1, 2, 3, and 4 equivalents of indenoisoquinoline compound 12 (B), 6 (D), and 17 (F). Conditions: 25° C., pH 7, 5 mM K⁺.

FIG. 15 Apparent binding affinities of the five indenoisoquinolines with MycPu22 determined by fluorescence-based binding assay. (A) Fluorescence intensity change of 3′-TAMRA-labeled MycPu22 DNA (0.5 nM) at 580 nm upon respective titration of six indenoisoquinolines. Conditions: 20° C., pH 7, 100 mM K+. (B) Apparent K_{d} values determined for six indenoisoquinolines. N.D. indicates that the value was not determined due to the negligible change of fluorescence signal. The apparent binding affinity K_{d} values were determined by fitting the data to a one-site specific binding model using GraphPad Prism software, with a simplified equation of ΔF_{obs}=ΔFₘₐₓ[L]_{T}/([L]_{T}+K_{d,app}), where ΔF represents the fluorescence intensity change of the indenoisoquinolines bound to MycPu22 DNA and [L]T represents the total ligand concentration.

FIG. 16A Binding selectivities of MycG4-interactive indenoisoquinolines. Competition fluorescence displacement experiments with increasing concentrations of unlabeled G4s and ds-DNA were added to 3′-TAMRA labeled MycPu22 (20 nM) mixed with 5 equivalents of indenoisoquinoline compound 13 (panel A). The normalized TAMRA fluorescence intensities at 580 nm were plotted as a function of molar ratio of G4 (in 3 G-tetrads) or calf thymus ds-DNA (in 11 bp) to labeled MycPu22. The vertical scale is the normalized fluorescence intensity recovery percentage. The fluorescence intensity of free 3′-TAMRA-labeled MycPu22 was defined as 100%, and the fluorescence intensity of a 1:5 mixture of 3′-TAMRA-labeled MycPu22 and indenoisoquinoline was defined as 0%. Conditions: 20° C., pH 7, 100 mM K⁺.

FIG. 16B Binding selectivities of MycG4-interactive indenoisoquinolines. Competition fluorescence displacement experiments with increasing concentrations of unlabeled G4s and ds-DNA were added to 3′-TAMRA labeled MycPu22 (20 nM) mixed with 5 equivalents of indenoisoquinoline compound 5 (panel B), and 9 (panel D). The normalized TAMRA fluorescence intensities at 580 nm were plotted as a function of molar ratio of G4 (in 3 G-tetrads) or calf thymus ds-DNA (in 11 bp) to labeled MycPu22. The vertical scale is the normalized fluorescence intensity recovery percentage. The fluorescence intensity of free 3′-TAMRA-labeled MycPu22 was defined as 100%, and the fluorescence intensity of a 1:5 mixture of 3′-TAMRA-labeled MycPu22 and indenoisoquinoline was defined as 0%. Conditions: 20° C., pH 7, 100 mM K⁺.

FIG. 16C Binding selectivities of MycG4-interactive indenoisoquinolines. Competition fluorescence displacement experiments with increasing concentrations of unlabeled G4s and ds-DNA were added to 3′-TAMRA labeled MycPu22 (20 nM) mixed with 5 equivalents of indenoisoquinoline compound 6 (panel C) and 12 (panel E). The normalized TAMRA fluorescence intensities at 580 nm were plotted as a function of molar ratio of G4 (in 3 G-tetrads) or calf thymus ds-DNA (in 11 bp) to labeled MycPu22. The vertical scale is the normalized fluorescence intensity recovery percentage. The fluorescence intensity of free 3′-TAMRA-labeled MycPu22 was defined as 100%, and the fluorescence intensity of a 1:5 mixture of 3′-TAMRA-labeled MycPu22 and indenoisoquinoline was defined as 0%. Conditions: 20° C., pH 7, 100 mM K⁺.

FIG. 17A Bar graphs showing the antiproliferation profiles (GI₅₀) of indenoisoquinolines 12 and 5 from the NCI-60 cancer cell line drug screen. MYC inhibition and topoisomerase I inhibition levels are shown at the top. Bar graphs are constructed for each compound, with bars depicting the deviation of individual cancer cell lines from the compound 4 mean(log₁₀ GI₅₀) value of −5.53. Compounds 12 and 5 with strong MYC inhibition and topoisomerase I inhibition show more potent anticancer activities compared to compounds 4 and 20. ND: GI₅₀ value not determined.

FIG. 17B Bar graphs showing the antiproliferation profiles (GI₅₀) of indenoisoquinolines 4 and 20 from the NCI-60 cancer cell line drug screen. MYC inhibition and topoisomerase I inhibition levels are shown at the top. Bar graphs are constructed for each compound, with bars depicting the deviation of individual cancer cell lines from the compound 4 mean(log₁₀ GI₅₀) value of −5.53. Compounds 12 and 5 with strong MYC inhibition and topoisomerase I inhibition show more potent anticancer activities compared to compounds 4 and 20. ND: GI₅₀ value not determined.

### DETAILED DESCRIPTION

While the concepts of the present disclosure are illustrated and described in detail herein, the descriptions are to be considered as exemplary and not restrictive in character; it being understood that only the illustrative embodiments are shown and described and that all changes and modifications that come within the spirit of the disclosure are desired to be protected.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 10%, within 5%, or within 1% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein the terms “indenoisoquinoline” and “(aza)indenoisoquinoline” refers to both indenoisoquinolines and azaindenoisoquinolines.

The term “substituted” as used herein refers to a functional group in which one or more hydrogen atoms contained therein are replaced by one or more non-hydrogen atoms. The term “functional group” or “substituent” as used herein refers to a group that can be or is substituted onto a molecule. Examples of substituents or functional groups include, but are not limited to, a halogen (e.g., F, Cl, Br, and I); an oxygen atom in groups such as hydroxyl groups, alkoxy groups, aryloxy groups, arylalkyloxy groups, oxo(carbonyl) groups, carboxyl groups including carboxylic acids, carboxylates, carboxamides, and carboxylate esters; acyl groups; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

The term “alkyl” as used herein refers to substituted or unsubstituted straight chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups, as described herein.

The term “alkenyl” as used herein refers to substituted or unsubstituted straight chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkynyl” as used herein refers to a substituted or unsubstituted, straight or branched chain carbon chain having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) containing at least one carbon-carbon triple bond.

The term “hydroxyalkyl” as used herein refers to alkyl groups as defined herein substituted with at least one hydroxyl (—OH) group.

The term “cycloalkyl” as used herein refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and car-3-enyl 1 groups, and fused rings such as, but not limited to, decalinyl, and the like.

The term “acyl” as used herein refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl cycloalkyl, cycloalkylalkyl, heterocyclyl, heterocyclylalkyl, heteroaryl, heteroarylalkyl group or the like. In the special case wherein the carbonyl carbon atom is bonded to a hydrogen, the group is a “formyl” group, an acyl group as the term is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning here. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The terms “aralkyl” and “arylalkyl” as used herein refer to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Arylalkenyl groups are alkenyl groups as defined herein in which a hydrogen or carbon bond of an alkyl group is replaced with a bond to an aryl group as defined herein.

The term “heterocyclyl” as used herein refers to substituted or unsubstituted aromatic and non-aromatic ring compounds containing 3 or more ring members, of which, one or more is a heteroatom such as, but not limited to, B, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. In some embodiments, heterocyclyl groups include heterocyclyl groups that include 3 to 8 carbon atoms (C₃-C₈), 3 to 6 carbon atoms (C₃-C₆) or 6 to 8 carbon atoms (C₆-C₈).

A heteroaryl ring is an embodiment of a heterocyclyl group. The phrase “heterocyclyl group” includes fused ring species including those that include fused aromatic and non-aromatic groups. Representative heterocyclyl groups include, but are not limited to pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, and benzimidazolinyl groups.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclylalkyl groups include, but are not limited to, furan-2-yl methyl, furan-3-yl methyl, pyridine-3-yl methyl, tetrahydrofuran-2-yl methyl, and indol-2-yl propyl.

The term “heteroarylalkyl” as used herein refers to alkyl groups as defined herein in which a hydrogen or carbon bond of an alkyl group is replaced with a bond to a heteroaryl group as defined herein.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can further include double or triple bonds, and can also include heteroatoms. For example, an allyloxy group is an alkoxy group within the meaning herein. A methoxyethoxy group is also an alkoxy group within the meaning herein, as is a methylenedioxy group in a context where two adjacent atoms of a structure are substituted therewith.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and RN wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, —CF(CH₃)₂ and the like.

The term “optionally substituted,” or “optional substituents,” as used herein, means that the groups in question are either unsubstituted or substituted with one or more of the substituents specified. When the groups in question are substituted with more than one substituent, the substituents may be the same or different. When using the terms “independently,” “independently are,” and “independently selected from” mean that the groups in question may be the same or different. Certain of the herein defined terms may occur more than once in the structure, and upon such occurrence each term shall be defined independently of the other.

The compounds described herein may contain one or more chiral centers, or may otherwise be capable of existing as multiple stereoisomers. It is to be understood that in one embodiment, the invention described herein is not limited to any particular stereochemical requirement, and that the compounds, and compositions, methods, uses, and medicaments that include them may be optically pure, or may be any of a variety of stereoisomeric mixtures, including racemic and other mixtures of enantiomers, other mixtures of diastereomers, and the like. It is also to be understood that such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. It is to be understood that in another embodiment, the invention described herein is not limited to any particular geometric isomer requirement, and that the compounds, and compositions, methods, uses, and medicaments that include them may be pure, or may be any of a variety of geometric isomer mixtures. It is also to be understood that such mixtures of geometric isomers may include a single configuration at one or more double bonds, while including mixtures of geometry at one or more other double bonds.

As used herein, the terms “salts” and “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic groups such as amines; and alkali or organic salts of acidic groups such as carboxylic acids. Pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic, and the like.

Pharmaceutically acceptable salts can be synthesized from the parent compound which contains a basic or acidic moiety by conventional chemical methods. In some instances, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., 1985, the disclosure of which is hereby incorporated by reference.

The term “solvate” means a compound, or a salt thereof, that further includes a stoichiometric or non-stoichiometric amount of solvent bound by non-covalent intermolecular forces. Where the solvent is water, the solvate is a hydrate.

Further, in each of the foregoing and following embodiments, it is to be understood that the formulae include and represent not only all pharmaceutically acceptable salts of the compounds, but also include any and all hydrates and/or solvates of the compound formulae or salts thereof. It is to be appreciated that certain functional groups, such as the hydroxy, amino, and like groups form complexes and/or coordination compounds with water and/or various solvents, in the various physical forms of the compounds. Accordingly, the above formulae are to be understood to include and represent those various hydrates and/or solvates. In each of the foregoing and following embodiments, it is also to be understood that the formulae include and represent each possible isomer, such as stereoisomers and geometric isomers, both individually and in any and all possible mixtures. In each of the foregoing and following embodiments, it is also to be understood that the formulae include and represent any and all crystalline forms, partially crystalline forms, and non-crystalline and/or amorphous forms of the compounds.

The term “pharmaceutically acceptable carrier” is art-recognized and refers to a pharmaceutically-acceptable material, composition or vehicle, such as a liquid or solid filler, diluent, excipient, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

As used herein, the term “administering” includes all means of introducing the compounds and compositions described herein to the patient, including, but are not limited to, oral (po), intravenous (iv), intramuscular (im), subcutaneous (sc), transdermal, inhalation, buccal, ocular, sublingual, vaginal, rectal, and the like. The compounds and compositions described herein may be administered in unit dosage forms and/or formulations containing conventional nontoxic pharmaceutically acceptable carriers, adjuvants, and vehicles.

Illustrative formats for oral administration include tablets, capsules, elixirs, syrups, and the like. Illustrative routes for parenteral administration include intravenous, intraarterial, intraperitoneal, epidural, intraurethral, intrasternal, intramuscular and subcutaneous, as well as any other art recognized route of parenteral administration.

Illustrative means of parenteral administration include needle (including microneedle) injectors, needle-free injectors and infusion techniques, as well as any other means of parenteral administration recognized in the art. Parenteral formulations are typically aqueous solutions which may contain excipients such as salts, carbohydrates and buffering agents (preferably at a pH in the range from about 3 to about 9), but, for some applications, they may be more suitably formulated as a sterile non-aqueous solution or as a dried form to be used in conjunction with a suitable vehicle such as sterile, pyrogen-free water. The preparation of parenteral formulations under sterile conditions, for example, by lyophilization, may readily be accomplished using standard pharmaceutical techniques well known to those skilled in the art. Parenteral administration of a compound is illustratively performed in the form of saline solutions or with the compound incorporated into liposomes. In cases where the compound in itself is not sufficiently soluble to be dissolved, a solubilizer such as ethanol can be applied.

The dosage of each compound of the claimed combinations depends on several factors, including: the administration method, the condition to be treated, the severity of the condition, whether the condition is to be treated or prevented, and the age, weight, and health of the person to be treated. Additionally, pharmacogenomic (the effect of genotype on the pharmacokinetic, pharmacodynamic or efficacy profile of a therapeutic) information about a particular patient may affect the dosage used.

It is to be understood that in the methods described herein, the individual components of a co-administration, or combination can be administered by any suitable means, contemporaneously, simultaneously, sequentially, separately or in a single pharmaceutical formulation. Where the co-administered compounds or compositions are administered in separate dosage forms, the number of dosages administered per day for each compound may be the same or different. The compounds or compositions may be administered via the same or different routes of administration. The compounds or compositions may be administered according to simultaneous or alternating regimens, at the same or different times during the course of the therapy, concurrently in divided or single forms.

The term “therapeutically effective amount” as used herein, refers to that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated. In one aspect, the therapeutically effective amount is that which may treat or alleviate the disease or symptoms of the disease at a reasonable benefit/risk ratio applicable to any medical treatment. However, it is to be understood that the total daily usage of the compounds and compositions described herein may be decided by the attending physician within the scope of sound medical judgment. The specific therapeutically-effective dose level for any particular patient will depend upon a variety of factors, including the disorder being treated and the severity of the disorder; activity of the specific compound employed; the specific composition employed; the age, body weight, general health, gender and diet of the patient: the time of administration, route of administration, and rate of excretion of the specific compound employed; the duration of the treatment; drugs used in combination or coincidentally with the specific compound employed; and like factors well known to the researcher, veterinarian, medical doctor or other clinician of ordinary skill.

Depending upon the route of administration, a wide range of permissible dosages are contemplated herein, including doses falling in the range from about 1 μg/kg to about 1 g/kg, 10 μg/kg to about 1 g/kg, 100 μg/kg to about 1 g/kg, 500 μg/kg to about 1 g/kg, 1 mg/kg to about 1 g/kg, 10 mg/kg to about 1 g/kg, 100 mg/kg to about 1 g/kg, 500 mg/kg to about 1 g/kg, 1 μg/kg to about 1 g/kg, 1 μg/kg to about 1 g/kg, 1 μg/kg to about 1 g/kg, 1 μg/kg to about 1 g/kg. The dosages may be single or divided, and may administered according to a wide variety of protocols, including q.d. (once a day), b.i.d. (twice a day), t.i.d. (three times a day), or even every other day, once a week, once a month, once a quarter, and the like. In each of these cases it is understood that the therapeutically effective amounts described herein correspond to the instance of administration, or alternatively to the total daily, weekly, month, or quarterly dose, as determined by the dosing protocol.

In addition to the illustrative dosages and dosing protocols described herein, it is to be understood that an effective amount of any one or a mixture of the compounds described herein can be determined by the attending diagnostician or physician by the use of known techniques and/or by observing results obtained under analogous circumstances. In determining the effective amount or dose, a number of factors are considered by the attending diagnostician or physician, including, but not limited to the species of mammal, including human, its size, age, and general health, the specific disease or disorder involved, the degree of or involvement or the severity of the disease or disorder, the response of the individual patient, the particular compound administered, the mode of administration, the bioavailability characteristics of the preparation administered, the dose regimen selected, the use of concomitant medication, and other relevant circumstances.

The term “patient” includes human and non-human animals such as companion animals (dogs and cats and the like) and livestock animals. Livestock animals are animals raised for food production. The patient to be treated is preferably a mammal, in particular a human being.

It has been discovered that potent anticancer activity of (aza)indenoisoquinolines can be correlated with strong MYC suppression and dual-targeting of topoisomerase I. It has also been discovered that the suppression of MYC expression and inhibition of topoisomerase I may be synergistic. It is believed that patients whose cancers are MYC-positive and which contain MYC G4 forming region(s) in the DNA of the cancer cell are good candidates for successful treatment of their cancers with (aza)indenoisoquinolines. As used herein, the term “MYC-positive,” indicates a cell that over-expresses the MYC protein compared to normal cells. It is understood that normal cells contain MYC. MYC-positive is used to indicate that the MYC protein levels are higher than in normal cells. These higher levels can be detected by commonly available procedures, such as IHC or western blot. Generally MYC protein levels in normal cells are very low and show as very faint band in a western blot, whereas in cancer cells that are “MYC-positive” a very clear band shows for the MYC protein in the western blot. As used herein, the phrase “a MYC-positive-cancer” indicates a cancer where the proliferation of the cancer cells is dependent or partially dependent on MYC.

As used herein, cancer cells are “MYC G4-positive” when the MYC promoter sequence in those cancer cells contains a nucleic acid sequence capable of forming a MYC G-quadruplex (MYC G4).

In some illustrative embodiments, the present disclosure relates to a method for selecting a patient for treatment and treating the patient with a compound of formula (I) or a pharmaceutically acceptable salt, hydrate, or solvate thereof; wherein the patient has a MYC-positive cancer comprising the steps of obtaining a sample of the patient's cancer; determining if the cancer cells in the sample are MYC-positive; and selecting the patient for treatment with the compound if the patient's cancer cells are MYC-positive; where the compound of formula (I) is

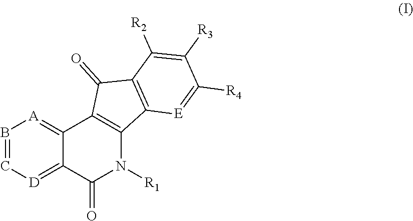

or a pharmaceutically acceptable salt, hydrate, or solvate thereof, wherein

A is N, CH, or CR; B is N, CH, or CR; C is N, CH, or CR; D is N, CH, or CR; E is N, CH, or CR, wherein R is a halo, azido, alkoxy, cyano, nitro, hydroxy, amino, thio, or a derivative thereof; or an alkyl, alkenyl, heteroalkyl, heteroalkenyl, heterocyclyl, cycloalkyl, cycloalkenyl, cycloheteroalkyl, cycloheteroalkenyl, aryl, arylalkyl, and arylalkenyl, each of which is optionally substituted;

R₁ is an alkyl, alkenyl, heteroalkyl, heteroalkenyl, heterocyclyl, hydroxyalkyl, hydroxyalkylaminoalkyl, and heterocyclylalkyl, cycloalkyl, cycloalkenyl, cycloheteroalkyl, cycloheteroalkenyl, aryl, arylalkyl, and arylalkenyl, each of which is optionally substituted;

R₂, R₃, and R₄ represent four substituents each independently selected from the group consisting of hydrogen, halo, azido, alkoxy, cyano, nitro, hydroxy, amino, thio, and derivatives thereof; or any two adjacent substituents that are taken together with the attached carbons to form an optionally substituted heterocycle, and each of other two substituents is defined as above.

In some illustrative embodiments, the present disclosure relates to a method for selecting and treating a patient if the patient's cancer cells are MYC-positive with a compound of formula (I) or a pharmaceutically acceptable salt, hydrate, or solvate thereof wherein R₁ is a C₁-C₁₂ alkyl, alkenyl, heteroalkyl, heteroalkenyl, hydroxyalkyl, hydroxyalkylaminoalkyl, and heterocyclylalkyl, (add a space here) or heterocyclyl, each of which is optionally substituted.

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein R₁ is

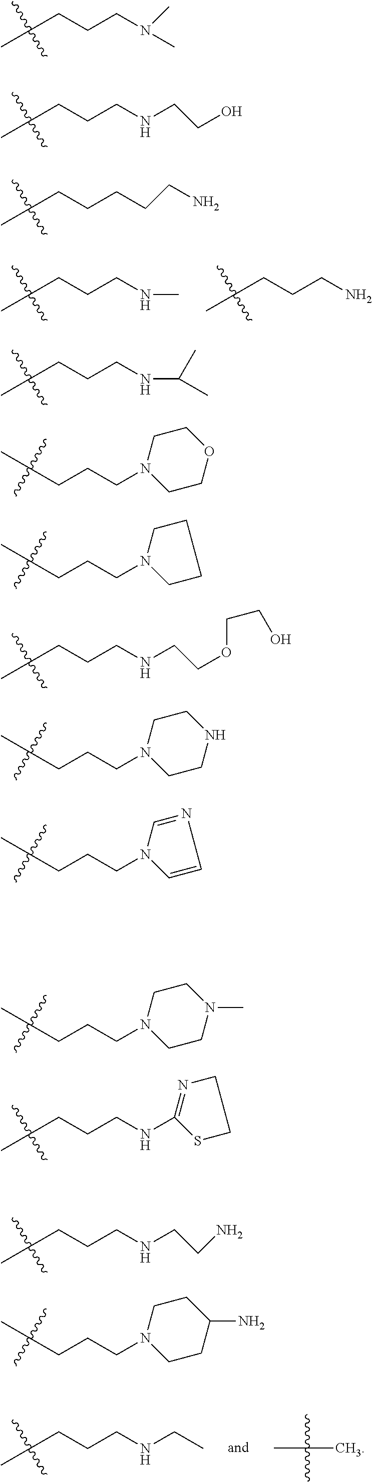

In some illustrative embodiments, the present disclosure relates to a method for selecting and treating a patient if the patient's cancer cells are MYC-positive with a compound of formula (I) or a pharmaceutically acceptable salt, hydrate, or solvate thereof wherein D is N.

In some illustrative embodiments, the present disclosure relates to a method for selecting and treating a patient if the patient's cancer cells are MYC-positive with a compound of formula (I) or a pharmaceutically acceptable salt, hydrate, or solvate thereof wherein E is N.

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein said compounds are selected from the following compound list (List 1).

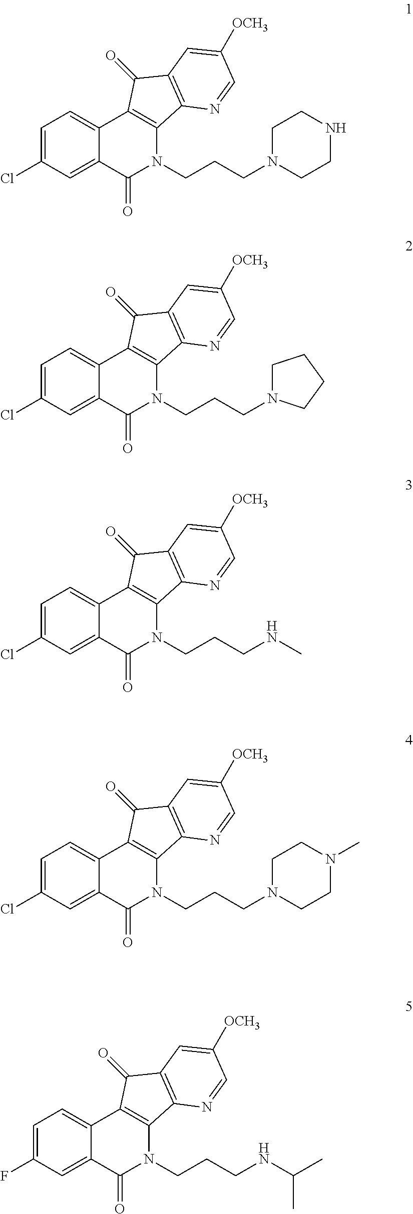 [SMILES: COc1cnc2c(c1)C(=O)c1c-2n(CCCN2CCNCC2)c(=O)c2cc(Cl)ccc12 ; COc1cnc2c(c1)C(=O)c1c-2n(CCCN2CCCC2)c(=O)c2cc(Cl)ccc12 ; CNCCCn1c2c(c3ccc(Cl)cc3c1=O)C(=O)c1cc(OC)cnc1-2 ; COc1cnc2c(c1)C(=O)c1c-2n(CCCN2CCN(C)CC2)c(=O)c2cc(Cl)ccc12 ; COc1cnc2c(c1)C(=O)c1c-2n(CCCNC(C)C)c(=O)c2cc(F)ccc12]

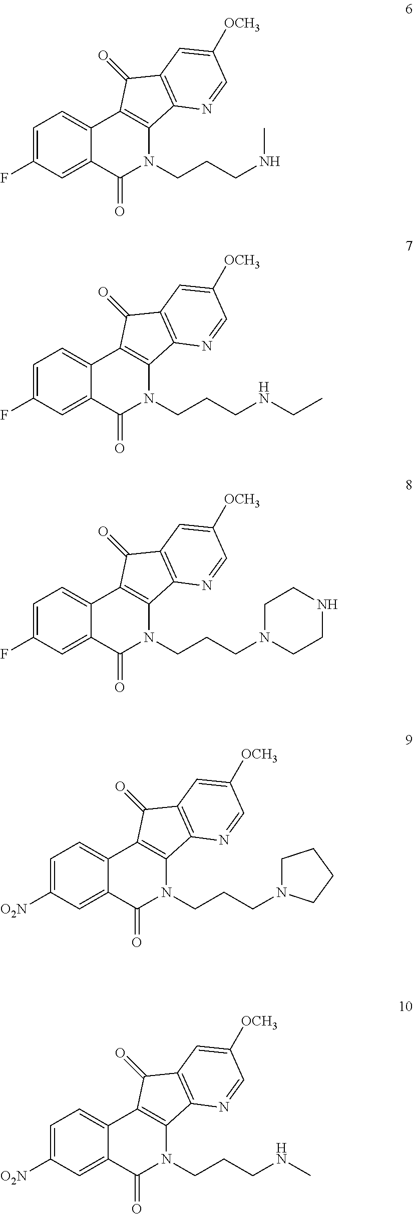 [SMILES: CNCCCn1c2c(c3ccc(F)cc3c1=O)C(=O)c1cc(OC)cnc1-2 ; CCNCCCn1c2c(c3ccc(F)cc3c1=O)C(=O)c1cc(OC)cnc1-2 ; COc1cnc2c(c1)C(=O)c1c-2n(CCCN2CCNCC2)c(=O)c2cc(F)ccc12 ; COc1cnc2c(c1)C(=O)c1c-2n(CCCN2CCCC2)c(=O)c2cc([N+](=O)[O-])ccc12 ; CNCCCn1c2c(c3ccc([N+](=O)[O-])cc3c1=O)C(=O)c1cc(OC)cnc1-2]

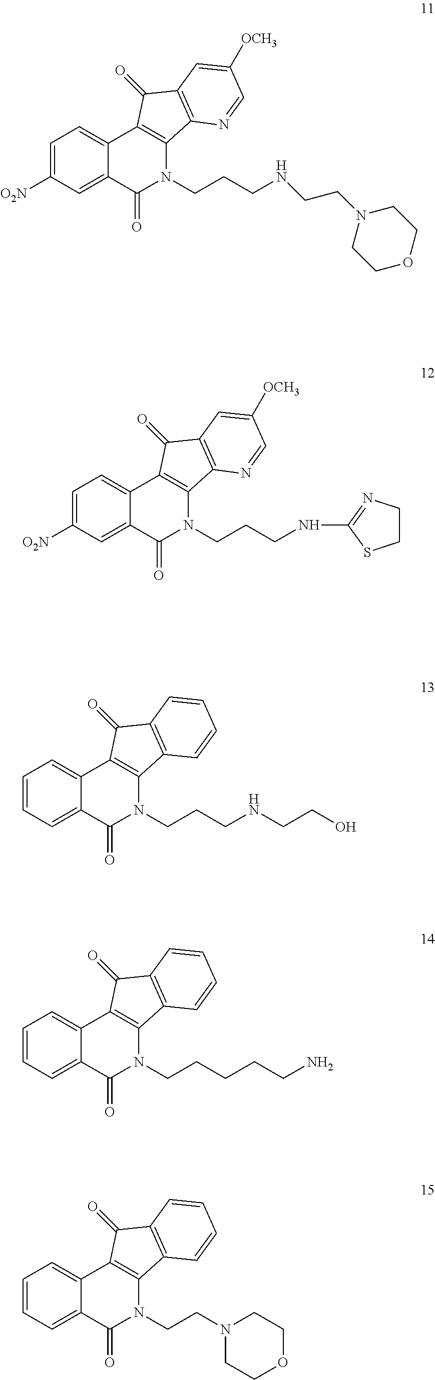 [SMILES: COc1cnc2c(c1)C(=O)c1c-2n(CCCNCCN2CCOCC2)c(=O)c2cc([N+](=O)[O-])ccc12 ; COc1cnc2c(c1)C(=O)c1c-2n(CCCNC2=NCCS2)c(=O)c2cc([N+](=O)[O-])ccc12 ; O=C1c2ccccc2-c2c1c1ccccc1c(=O)n2CCCNCCO ; NCCCCCn1c2c(c3ccccc3c1=O)C(=O)c1ccccc1-2 ; O=C1c2ccccc2-c2c1c1ccccc1c(=O)n2CCN1CCOCC1]

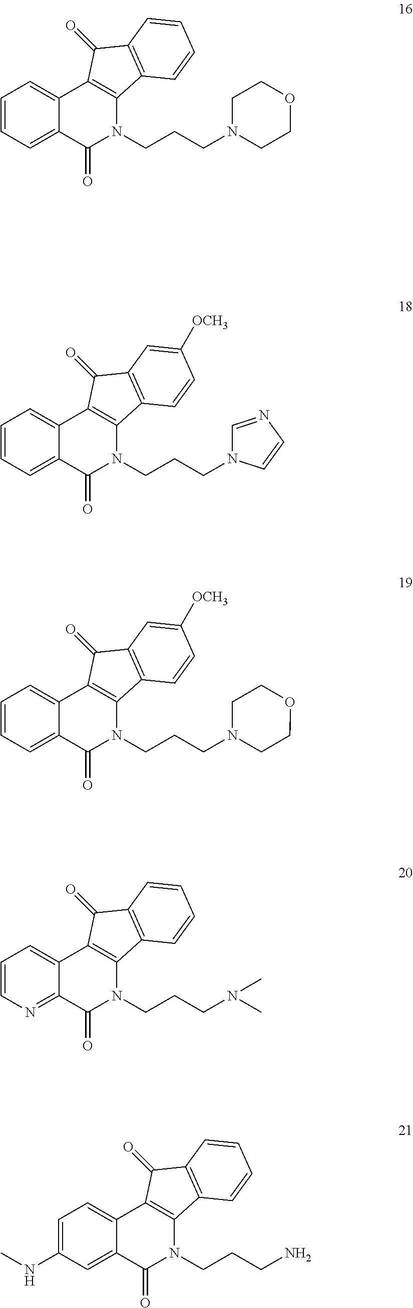

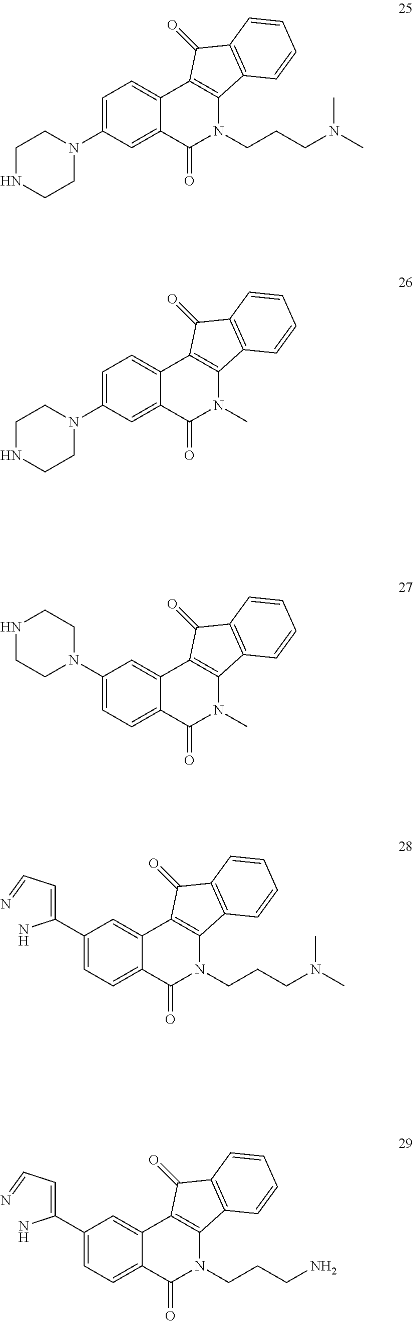 [SMILES: CN(C)CCCn1c2c(c3ccc(N4CCNCC4)cc3c1=O)C(=O)c1ccccc1-2 ; Cn1c2c(c3ccc(N4CCNCC4)cc3c1=O)C(=O)c1ccccc1-2 ; Cn1c2c(c3cc(N4CCNCC4)ccc3c1=O)C(=O)c1ccccc1-2 ; CN(C)CCCn1c2c(c3cc(/C(N)=C/C=N)ccc3c1=O)C(=O)c1ccccc1-2 ; N=C/C=C(\N)c1ccc2c(=O)n(CCCN)c3c(c2c1)C(=O)c1ccccc1-3]

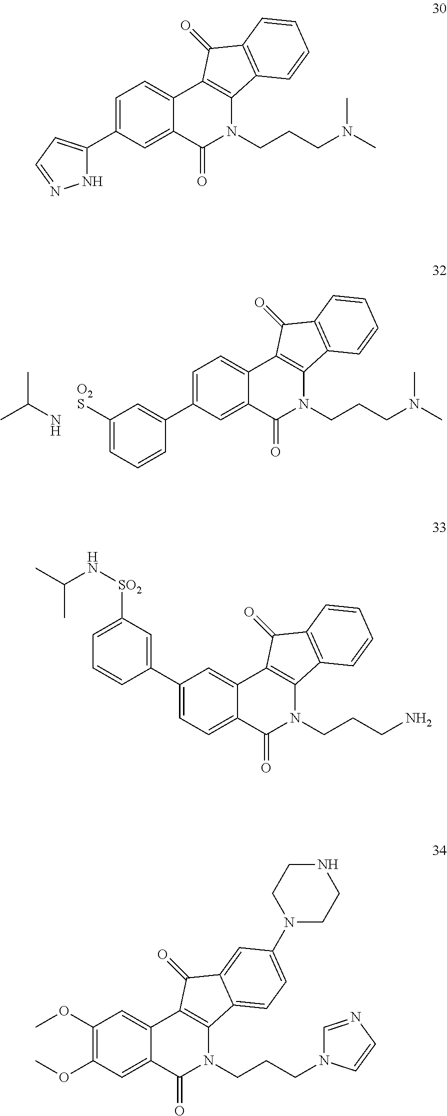

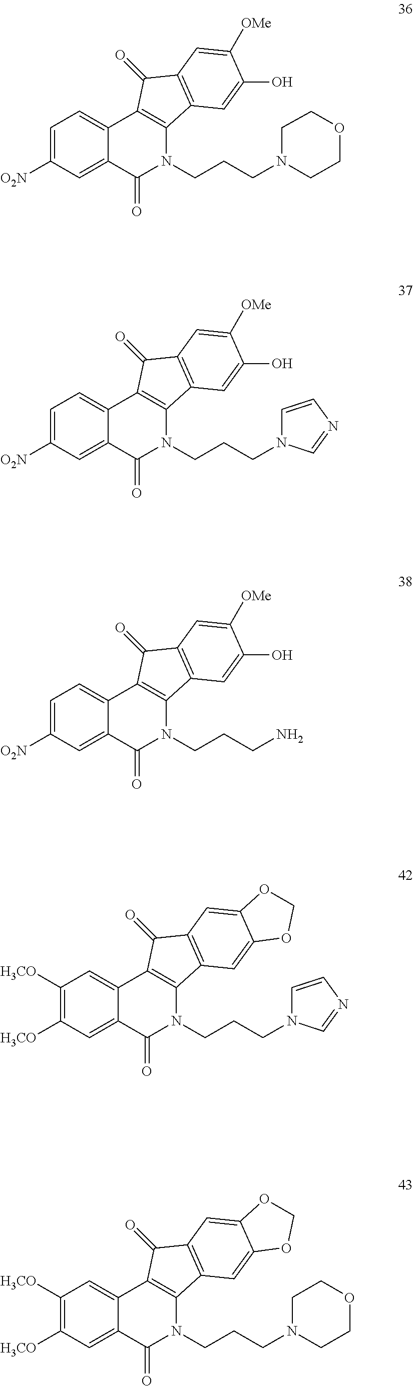 [SMILES: COc1cc2c(cc1O)-c1c(c3ccc([N+](=O)[O-])cc3c(=O)n1CCCN1CCOCC1)C2=O ; COc1cc2c(cc1O)-c1c(c3ccc([N+](=O)[O-])cc3c(=O)n1CCCn1ccnc1)C2=O ; COc1cc2c(cc1O)-c1c(c3ccc([N+](=O)[O-])cc3c(=O)n1CCCN)C2=O ; COc1cc2c3c(n(CCCn4ccnc4)c(=O)c2cc1OC)-c1cc2c(cc1C3=O)OCO2 ; COc1cc2c3c(n(CCCN4CCOCC4)c(=O)c2cc1OC)-c1cc2c(cc1C3=O)OCO2]

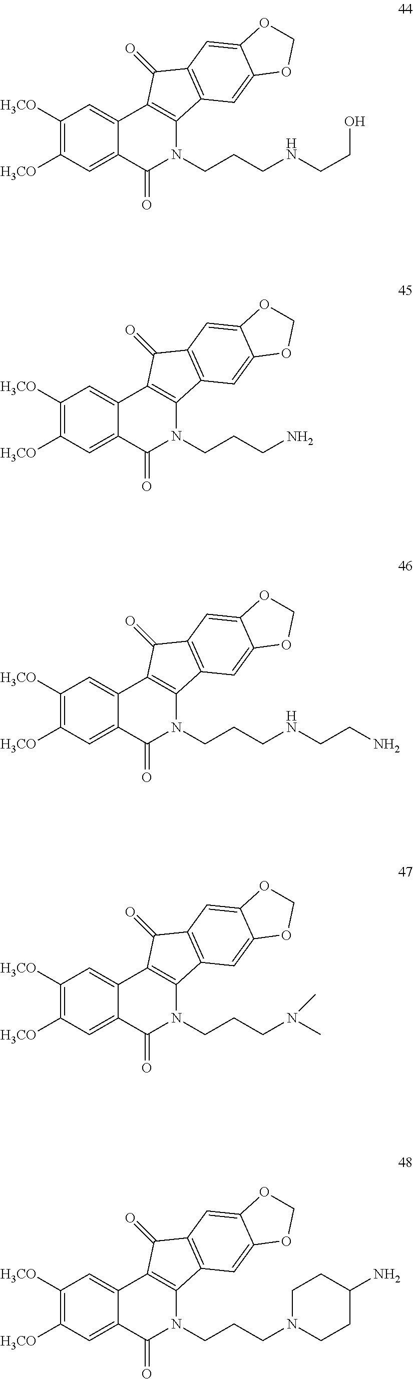 [SMILES: COc1cc2c3c(n(CCCNCCO)c(=O)c2cc1OC)-c1cc2c(cc1C3=O)OCO2 ; COc1cc2c3c(n(CCCN)c(=O)c2cc1OC)-c1cc2c(cc1C3=O)OCO2 ; COc1cc2c3c(n(CCCNCCN)c(=O)c2cc1OC)-c1cc2c(cc1C3=O)OCO2 ; COc1cc2c3c(n(CCCN(C)C)c(=O)c2cc1OC)-c1cc2c(cc1C3=O)OCO2 ; COc1cc2c3c(n(CCCN4CCC(N)CC4)c(=O)c2cc1OC)-c1cc2c(cc1C3=O)OCO2]

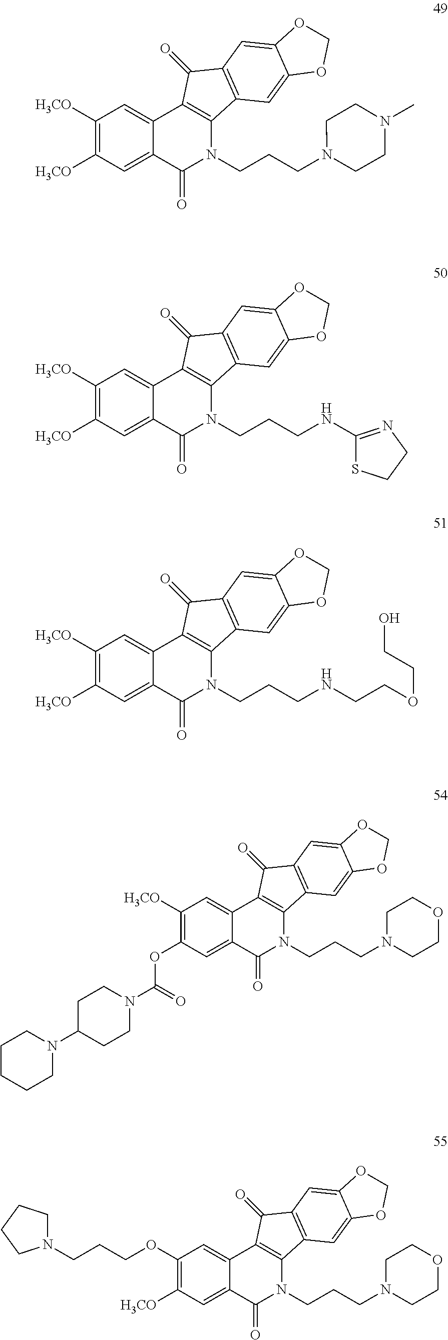 [SMILES: COc1cc2c3c(n(CCCN4CCN(C)CC4)c(=O)c2cc1OC)-c1cc2c(cc1C3=O)OCO2 ; COc1cc2c3c(n(CCCNC4=NCCS4)c(=O)c2cc1OC)-c1cc2c(cc1C3=O)OCO2 ; COc1cc2c3c(n(CCCNCCOCCO)c(=O)c2cc1OC)-c1cc2c(cc1C3=O)OCO2 ; COc1cc2c3c(n(CCCN4CCOCC4)c(=O)c2cc1OC(=O)N1CCC(N2CCCCC2)CC1)-c1cc2c(cc1C3=O)OCO2 ; COc1cc2c(=O)n(CCCN3CCOCC3)c3c(c2cc1OCCCN1CCCC1)C(=O)c1cc2c(cc1-3)OCO2]

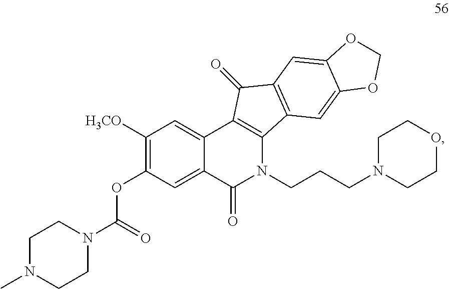 [SMILES: COc1cc2c3c(n(CCCN4CCOCC4)c(=O)c2cc1OC(=O)N1CCN(C)CC1)-c1cc2c(cc1C3=O)OCO2]

or a pharmaceutically acceptable salt, hydrate, or solvate thereof.

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein the compound of formula (I) is selected from the group consisting of

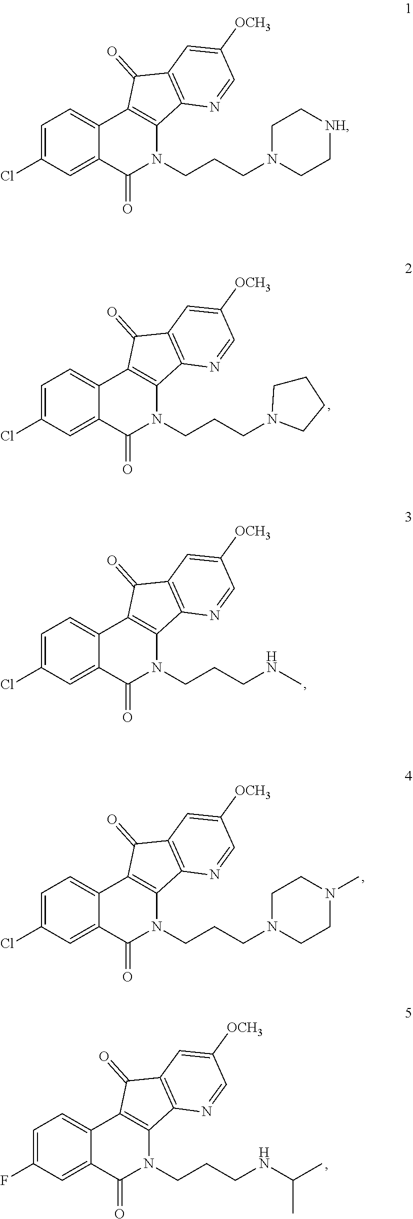 [SMILES: COc1cnc2c(c1)C(=O)c1c-2n(CCCN2CCNCC2)c(=O)c2cc(Cl)ccc12 ; COc1cnc2c(c1)C(=O)c1c-2n(CCCN2CCCC2)c(=O)c2cc(Cl)ccc12 ; CNCCCn1c2c(c3ccc(Cl)cc3c1=O)C(=O)c1cc(OC)cnc1-2 ; COc1cnc2c(c1)C(=O)c1c-2n(CCCN2CCN(C)CC2)c(=O)c2cc(Cl)ccc12 ; COc1cnc2c(c1)C(=O)c1c-2n(CCCNC(C)C)c(=O)c2cc(F)ccc12]

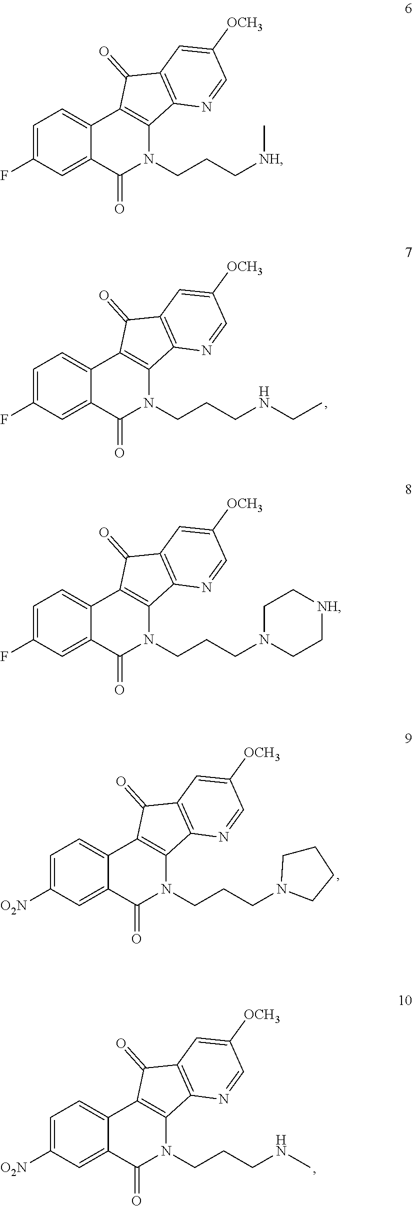 [SMILES: CNCCCn1c2c(c3ccc(F)cc3c1=O)C(=O)c1cc(OC)cnc1-2 ; CCNCCCn1c2c(c3ccc(F)cc3c1=O)C(=O)c1cc(OC)cnc1-2 ; COc1cnc2c(c1)C(=O)c1c-2n(CCCN2CCNCC2)c(=O)c2cc(F)ccc12 ; COc1cnc2c(c1)C(=O)c1c-2n(CCCN2CCCC2)c(=O)c2cc([N+](=O)[O-])ccc12 ; CNCCCn1c2c(c3ccc([N+](=O)[O-])cc3c1=O)C(=O)c1cc(OC)cnc1-2]

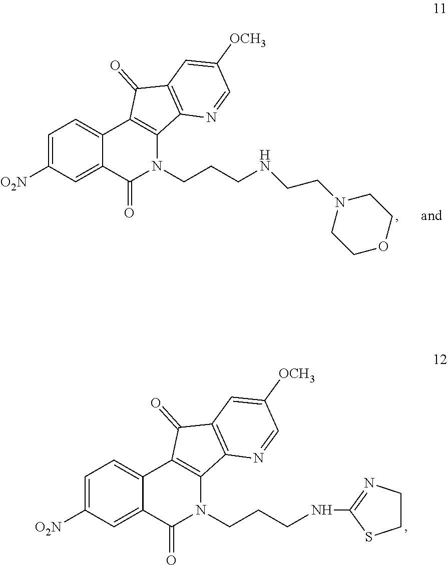 [SMILES: COc1cnc2c(c1)C(=O)c1c-2n(CCCNCCN2CCOCC2)c(=O)c2cc([N+](=O)[O-])ccc12 ; COc1cnc2c(c1)C(=O)c1c-2n(CCCNC2=NCCS2)c(=O)c2cc([N+](=O)[O-])ccc12]

or a pharmaceutically acceptable salt, hydrate, or solvate thereof

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein said compounds are selected from the group consisting of

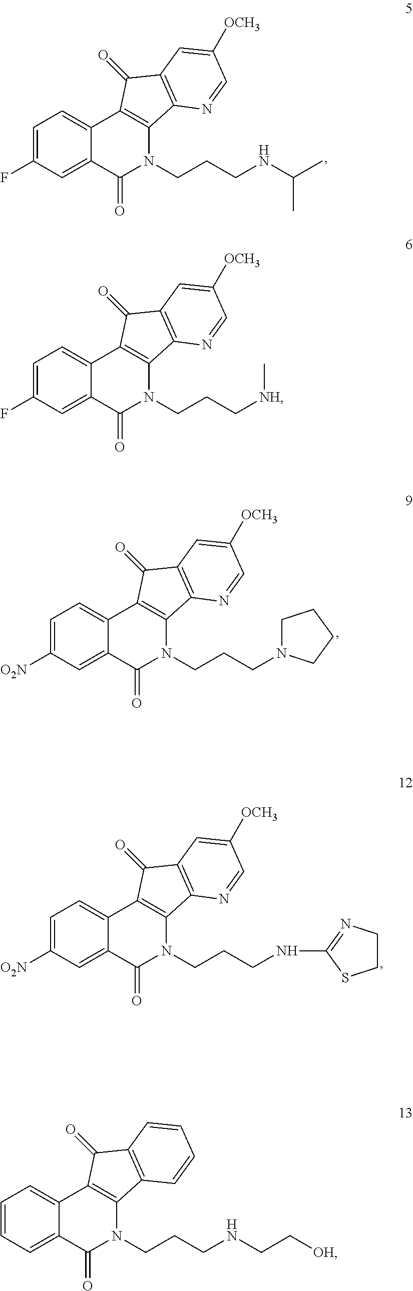 [SMILES: COc1cnc2c(c1)C(=O)c1c-2n(CCCNC(C)C)c(=O)c2cc(F)ccc12 ; CNCCCn1c2c(c3ccc(F)cc3c1=O)C(=O)c1cc(OC)cnc1-2 ; COc1cnc2c(c1)C(=O)c1c-2n(CCCN2CCCC2)c(=O)c2cc([N+](=O)[O-])ccc12 ; COc1cnc2c(c1)C(=O)c1c-2n(CCCNC2=NCCS2)c(=O)c2cc([N+](=O)[O-])ccc12 ; O=C1c2ccccc2-c2c1c1ccccc1c(=O)n2CCCNCCO]

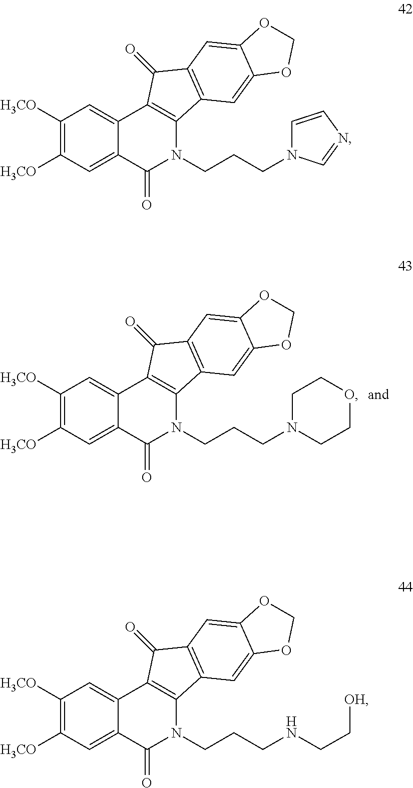 [SMILES: COc1cc2c3c(n(CCCn4ccnc4)c(=O)c2cc1OC)-c1cc2c(cc1C3=O)OCO2 ; COc1cc2c3c(n(CCCN4CCOCC4)c(=O)c2cc1OC)-c1cc2c(cc1C3=O)OCO2 ; COc1cc2c3c(n(CCCNCCO)c(=O)c2cc1OC)-c1cc2c(cc1C3=O)OCO2]

or a pharmaceutically acceptable salt, hydrate, or solvate thereof.

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein said compound is a human topoisomerase I inhibitor.

In some illustrative embodiments, the present disclosure relates to a method for selecting and treating a patient if the patient's cancer cells are MYC-positive with a compound of formula (I) or a pharmaceutically acceptable salt, hydrate, or solvate thereof, wherein the compound of formula (I) are selected from the group consisting of

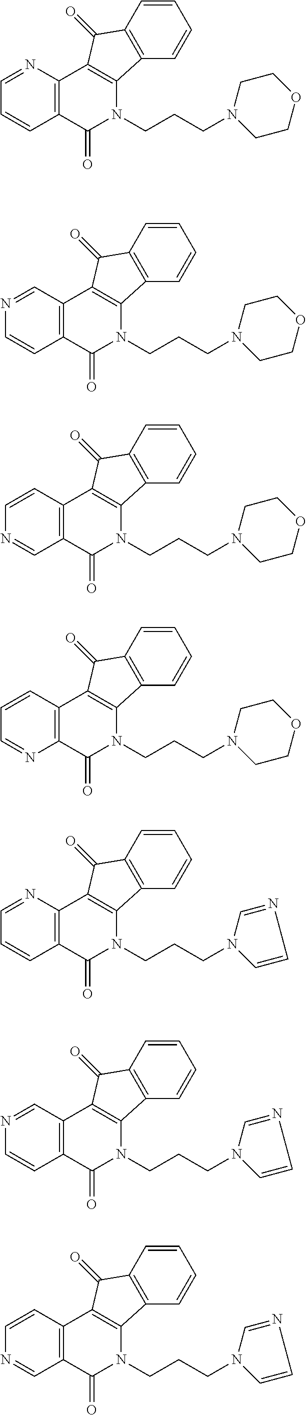

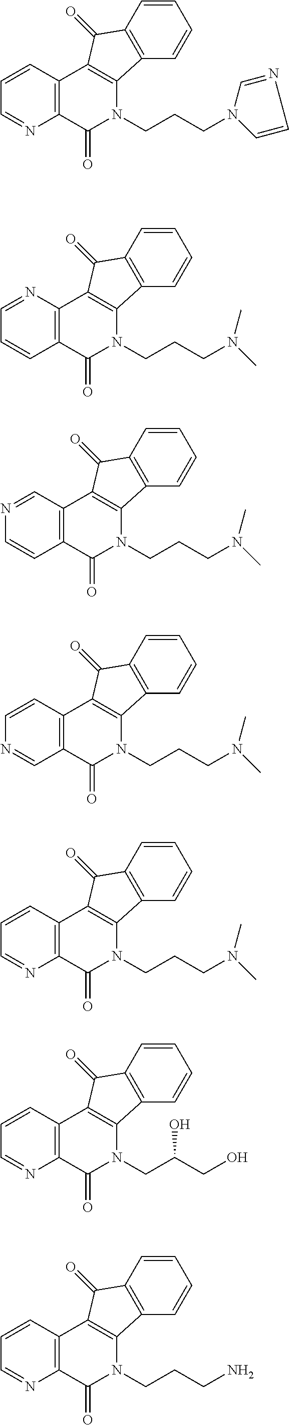

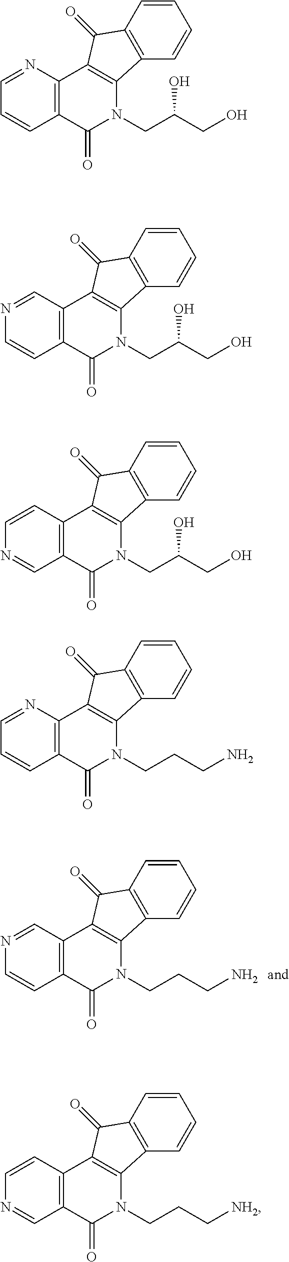 [SMILES: O=C1c2ccccc2-c2c1c1ncccc1c(=O)n2C[C@H](O)CO ; O=C1c2ccccc2-c2c1c1cnccc1c(=O)n2C[C@H](O)CO ; O=C1c2ccccc2-c2c1c1ccncc1c(=O)n2C[C@H](O)CO ; NCCCn1c2c(c3ncccc3c1=O)C(=O)c1ccccc1-2 ; NCCCn1c2c(c3cnccc3c1=O)C(=O)c1ccccc1-2 ; NCCCn1c2c(c3ccncc3c1=O)C(=O)c1ccccc1-2]

or a pharmaceutically acceptable salt, hydrate, or solvate thereof.

In some illustrative embodiments, the present disclosure relates to a method for selecting and treating a patient if the patient's cancer cells are MYC-positive with a compound of formula (II):

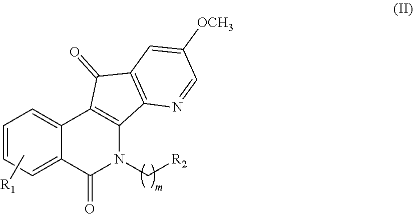

or a pharmaceutically acceptable salt, hydrate, or solvate thereof, wherein m is 3, R¹ is 3-Cl or 3-F, and R² is selected from the group consisting of

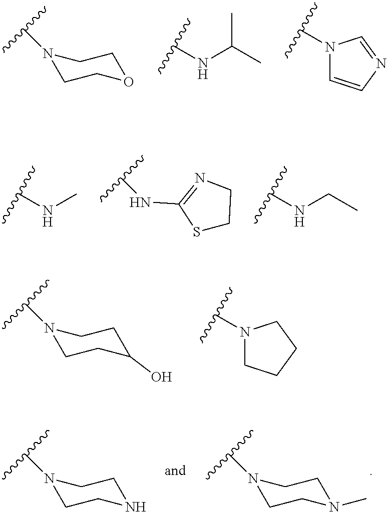

In some illustrative embodiments, the present disclosure relates to a method for selecting and treating a patient if the patient's cancer cells are MYC-positive with a composition comprising a compound of formula (I) or a pharmaceutically acceptable salt, hydrate, or solvate thereof, wherein said compounds are a human topoisomerase I inhibitor and wherein said compounds block the activities of MYC oncogene through binding the MYC G-quadruplex.

In any of the preceding embodiments disclosed herein, the present disclosure further relates to a method for selecting and treating the patient if the patient's cancer cells are MYC-positive and contain a MYC promoter G-quadruplex (MYC G4), i.e. are MYC G4-positive, the method further comprising the step of determining if the cancer cells in the sample are MYC G4-positive.

Herein, using fluorescence resonance energy transfer (FRET) assays, nuclear magnetic resonance (NMR), fluorescence-based binding assay and competition fluorescence displacement assay, circular dichroism (CD) spectroscopy, and gel electromobility shift assay (EMSA), it has been shown that a large number of anticancer indenoisoquinolines strongly bind and stabilize MYC G4 in vitro. Using cell-based western blotting and quantitative reverse transcription-polymerase chain reaction (qRT-PCR) assays, it is shown that MYC G4-interactive indenoisoquinolines lower MYC mRNA and protein levels in vivo, indicating that targeting the MYC promoter G4 to downregulate MYC may be a mechanism of action for the anticancer activities of these particular indenoisoquinolines. Furthermore, some active indenoisoquinolines show both MYC downregulation and topoisomerase I inhibition, suggesting that dual-targeting of MycG4 and topoisomerase I could be a potential strategy for anticancer drug development.

### Results and Discussion

### Indenoisoquinolines can Induce and Stabilize MYC G4

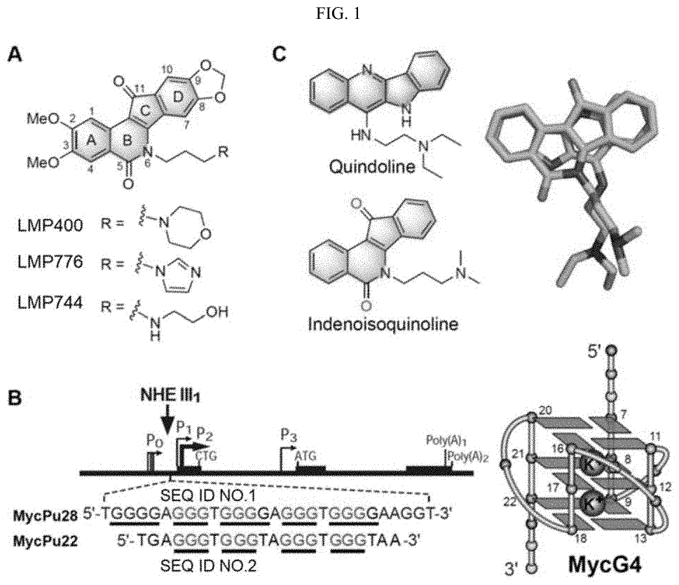

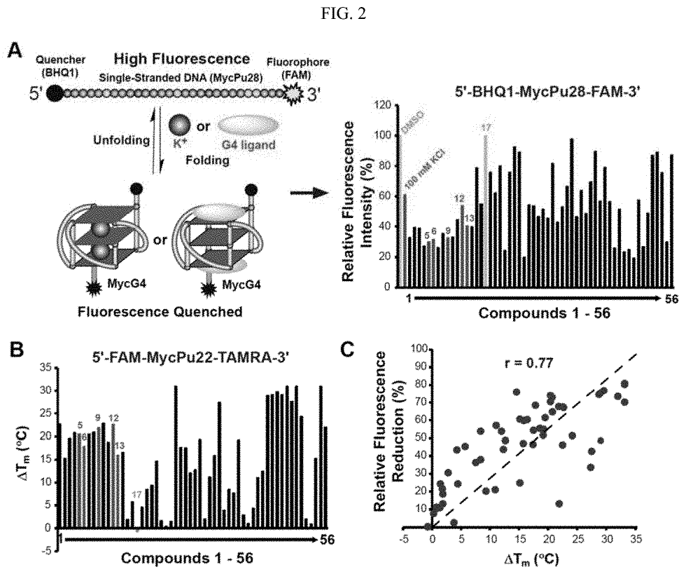

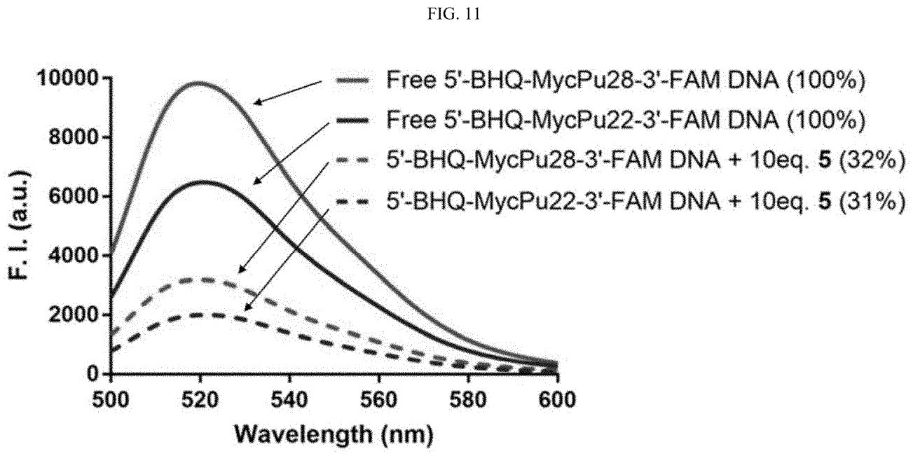

To examine whether the indenoisoquinolines could induce and stabilize the MYC G4 (MycG4), a FRET-quenching assay was conducted on indenoisoquinoline compounds. The full-length MYC promoter NHE III₁ G4 DNA (MycPu28, FIG. 1B) was labeled with FAM (6-fluorescein) on the 3′-end and BHQ-1 (Black Hole-1 quencher) on the 5′-end (FIG. 2A left). The MycG4 structure adopted by MycPu22 (FIG. 1B) is the major conformation formed by the wild-type MycPu28 in K⁺ solution.^{32, 43, 45} MycPu28 was used for the FRET-quenching screening assay because it has higher FAM-fluorescence than MycPu22 in the unfolded form due to the longer distance between the FAM and BHQ quencher, and thus provided greater range for screening (FIG. 11). We confirmed that very similar FRET-quenching effects were observed for MycPu22 and MycPu28 upon compound binding and G4-stabilization (FIG. 11). The stable formation of G-quadruplexes requires the presence of K⁺ or Na⁺ cations in solution, with a preference of K⁺ (FIG. 1B). In the absence of K⁺, the MycPu28 is in the extended single-stranded form with its two ends far apart and shows high FAM-fluorescence (FIG. 2A left). In the presence of 100 mM K⁺, the G4 is folded and the FAM-fluorescence is quenched because the quencher and fluorophore at the two ends are in closer proximity (FIG. 2A left). Alternatively, the addition of G4-stabilizing ligands can induce G4 formation in the absence of K⁺ and thereby lead to quenching FAM-fluorescence (FIG. 2A left).

56 indenoisoquinoline compounds (List 1, shown above) were examined using this FRET-quenching assay (FIG. 2A). K⁺ buffer (100 mM) was used as a positive control, which decreased FAM-fluorescence by 39%. It was found that 37 compounds decreased the FAM-fluorescence by more than 39%, indicating that these indenoisoquinolines can induce and stabilize MycG4. Some indenoisoquinolines decreased FAM-fluorescence more than 100 mM K⁺, which is likely due to greater stabilization of MycG4 or its flanking structures. However, it is possible that some indenoisoquinolines may interact with the FAM fluorophore directly to quench the FAM-fluorescence.

To confirm the stabilizing effect of indenoisoquinolines on MycG4, the Tₘ values of MycG4 were measured in the presence of indenoisoquinoline compounds in 10 mM K⁺ using dual-3′-FAM- and 5′-TAMRA-labeled MycPu22 DNA by FRET-melting experiments. MycPu22 DNA forms a single MycG4 structure and was used for NMR structure determination (FIG. 1B, right).⁴³ Therefore, MycPu22 provides the best molecular system for MycG4 and was used in all the subsequent experiments. 10 mM K⁺ was used in the FRET-melting experiments because the melting temperature of MycG4 at 100 mM K⁺ is above 90° C., making it impossible to determine an accurate melting temperature upon compound addition.⁴⁹ The FRET-melting results showed that forty-four of the fifty-six indenoisoquinolines increased the Tₘ values of MycG4 by more than 5° C. (FIG. 2B). A clear positive correlation was observed between the indenoisoquinolines' ability to induce MycG4 formation and to increase its thermal stability (FIG. 2C).

### Indenoisoquinolines can Lower MYC Levels in Cancer Cells

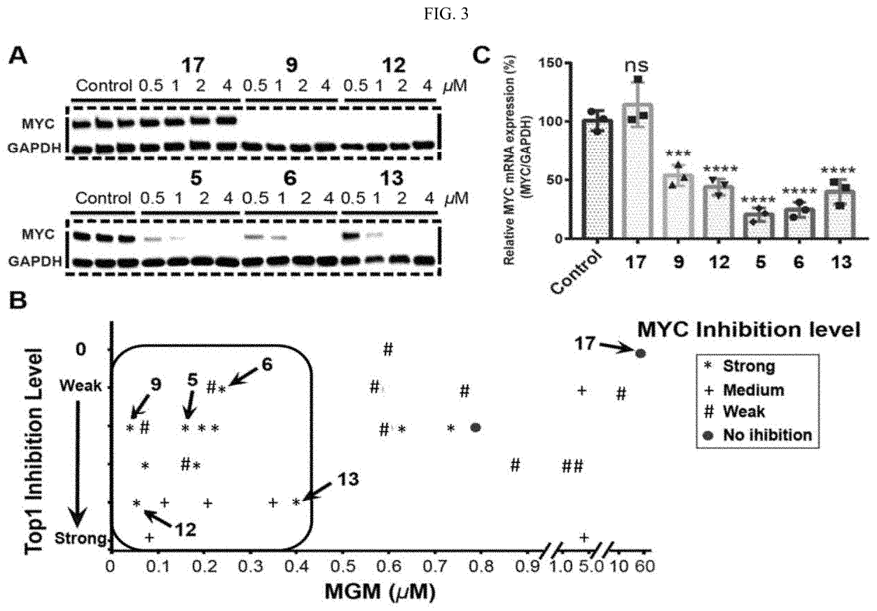

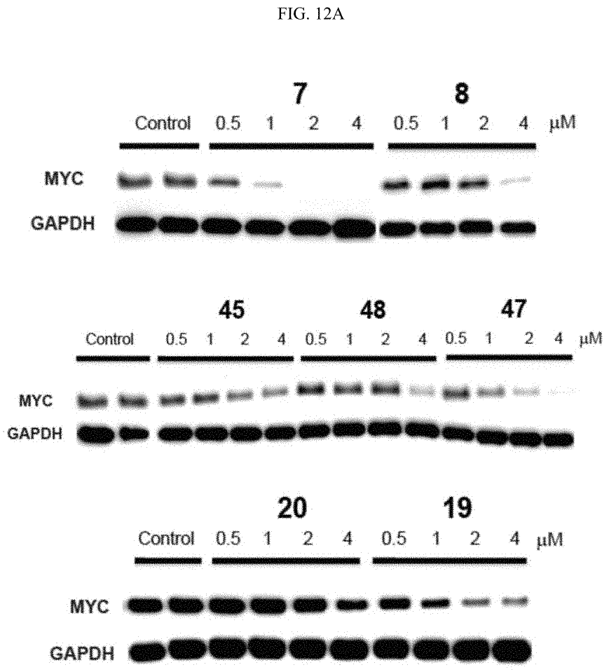

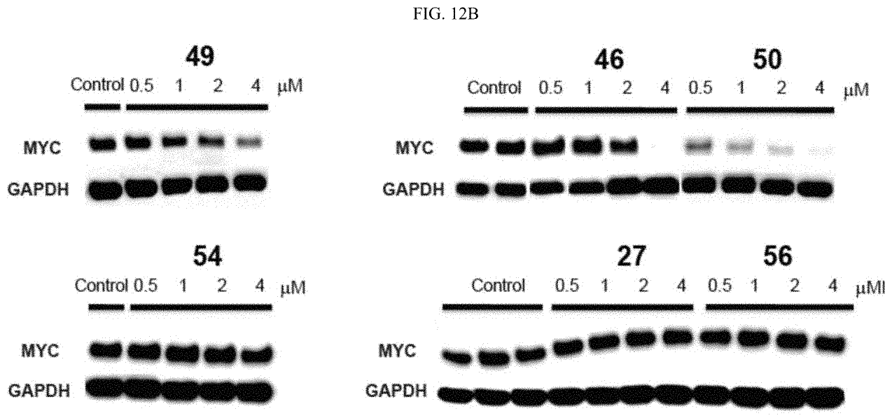

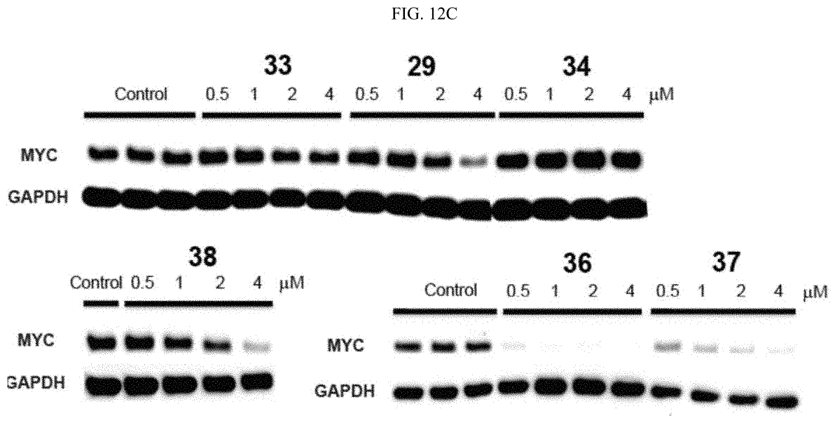

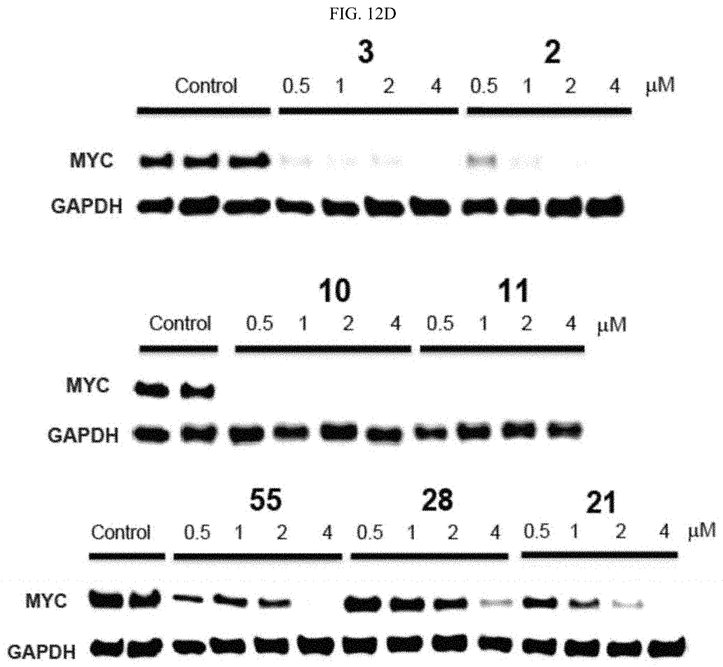

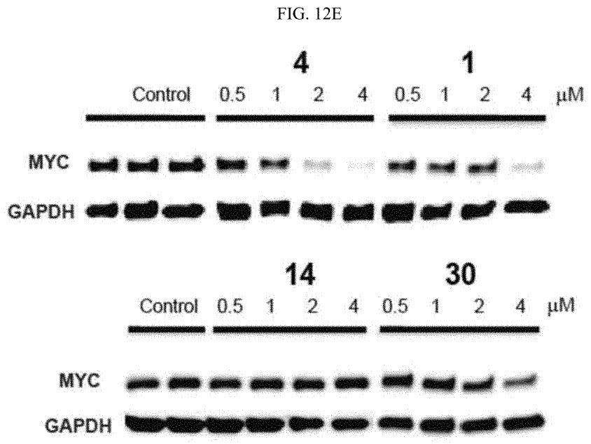

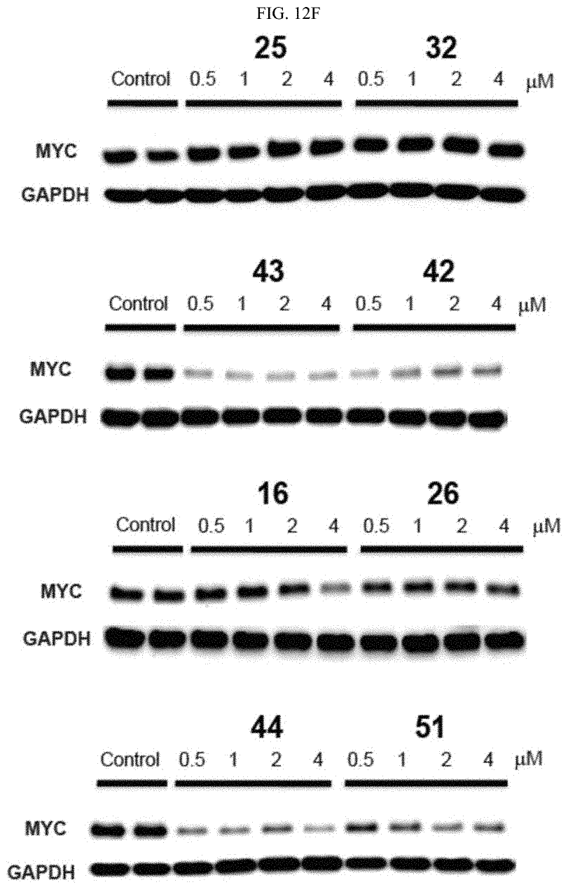

G-Quadruplex formed in the MYC promoter was found to function as a transcriptional silencer.³²⁻³⁴ To determine the effects of indenoisoquinolines on the MYC protein level, a western blotting experiment was carried out using MCF-7 breast cancer cells treated with 44 indenoisoquinolines that increased the Tₘ value of MycG4 by more than 5° C. MCF-7 cells were incubated with each compound at four concentrations (0.5, 1, 2, and 4 μM) for 24 hours, and the MYC protein levels were measured (FIG. 3A and FIGS. 12 A to 12 F).

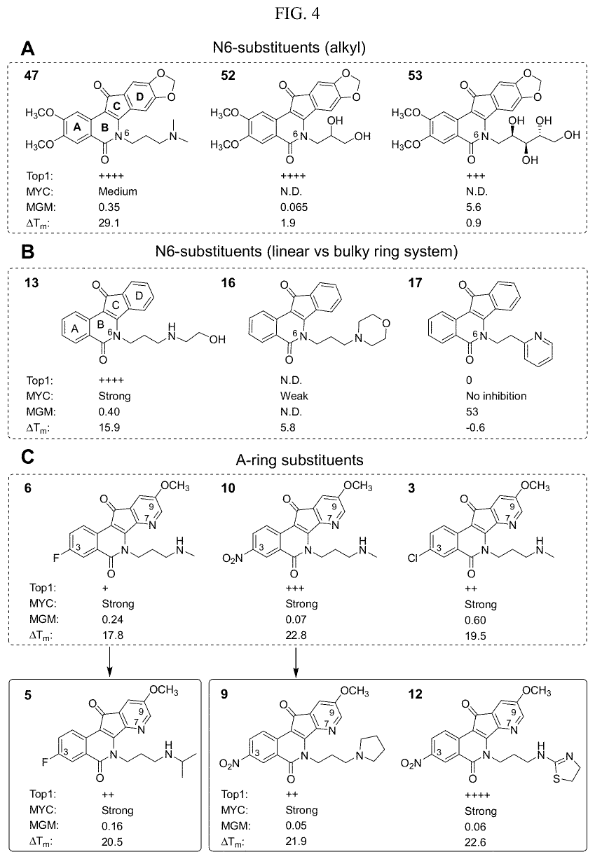

The human topoisomerase I inhibitory activities of the 44 indenoisoquinolines have been previously determined.^{3, 6-9, 15, 50-52} Of the 44 compounds tested for their cytotoxicities in the NCI-60 cancer cell lines, the 31 most potent compounds had their mean graph midpoint (MGM) values determined based on the GI₅₀ values obtained from the NCI-60 cancer cell line drug screen (Table 3).⁵³⁻⁵⁵ The topoisomerase I inhibitory activities were plotted against the anticancer activities of these 31 compounds (FIG. 3B). Some of the more active compounds (with MGM values <0.5 μM) showed strong topoisomerase I inhibition. However, many of the active compounds were not strong topoisomerase I inhibitors. The MYC inhibition activities of these compounds were ranked in four groups, i.e., strong, medium, weak, and no inhibition (FIG. 3B). Significantly, strong MYC inhibition was concentrated in compounds with potent anticancer activities, including those showing weak topoisomerase I inhibitory activity (FIG. 3B). We selected compounds 5, 6, 9, 12 and 13 for further investigation as they showed clear MYC-inhibitory effect (FIGS. 3B and 4). Compound 17 was used as a negative control (FIGS. 3B and 4).

To confirm the effect on the transcription of the MYC gene in cancer cells by the six selected indenoisoquinoline compounds, the MYC mRNA levels in MCF-7 cancer cells were measured by qRT-PCR. Consistent with the western blotting data, all five MYC-inhibiting compounds significantly lowered MYC mRNA levels at 6 hours post the treatments with 1 μM indenoisoquinolines. The negative control compound 17 showed no reduction of MYC mRNA level (FIG. 3C).

TABLE 1
The DNA sequences and primers used in this study.

| | | Sequence |
|---|---|---|
| Sequence Name | DNA Sequence (5′ to 3′) | ID No |
| MycPu28 | TGGGGAGGGTGGGGAGGGTGGGG | SEQ ID NO. 1 |
| | AAGGT | |
| MycPu22 | TGAGGGTGGG TAGGGTGGGTAA | SEQ ID NO. 2 |
| K-RasG4 | AGGGCGGTGTGGGAAGAGGGAAG | SEQ ID NO. 3 |
| | AGGGGGAGG | |
| Telomeric G4 | TTAGGGTTAGGGTTAGGGTTAGG | SEQ ID NO. 4 |
| | GTT | |
| qRT-PCR | | |
| Primers | | |
| MYC Forward | GCTGCTTAGACGCTGGATT | SEQ ID NO. 5 |
| MYC Reverse | TCCTCCTCGTCGCAGTAGA | SEQ ID NO. 6 |
| GAPDH Forward | CATGAGAAGTATGACAACAGCCT | SEQ ID NO. 7 |
| GAPDH Reverse | AGTCCTTCCACGATACCAAAGT | SEQ ID NO. 8 |

TABLE 2
Competitor binding affinities (Kᵢ) of the five indenoisoquinolines
determined by competition fluorescence displacement experiments.

| | Kᵢ (nM) | | | | |
|---|---|---|---|---|---|
| | | | | Telomeric | calf thymus |
| Compound | MycPu22 | MycPu28 | K-RasG4 | G4 | dsDNA^{a} |
| 5 | 16 | 18 | 21 | 164 | ~11000 |
| 6 | 40 | 33 | 32 | 286 | ~12000 |
| 9 | 12 | 11 | 11 | 28 | 5171 |
| 12 | 7 | 7 | 7 | 14 | 3624 |
| 13 | 26 | 19 | 23 | 146 | 2138 |

^{a}The Kᵢ value of the calf thymus ds-DNA refers to the base-pair concentration.

TABLE 3
MYC inhibition, Top1 inhibition, and GI50 values (pM) of the 29 indenoisoquinolines.

| Compound No. | 1 | 2 | 3 | 4 | 5 | 6 | 7 | 8 |
|---|---|---|---|---|---|---|---|---|
| MYC Inhibition^{a} | + | +++ | +++ | ++ | +++ | +++ | +++ | + |
| Top1 Inhibiton^{b} | + | ++ | ++ | + | ++ | + | ++ | + |
| MGM (uM)^{c} | 0.580 | 0.630 | 0.600 | 2.900 | 0.165 | 0.240 | 0.220 | 0.218 |
| Cancer Cell Lines | Antiproliferative Activities [GI50 (μM)]^{d} | | | | | | | |
| Leukemia | | | | | | | | |
| CCRF-CEM | 0.08 | 0.03 | 0.06 | 0.53 | 0.11 | 0.26 | 0.16 | 0.04 |
| HL-60(TB) | 0.11 | 6.23 | 0.60 | 18.00 | 0.16 | 0.22 | 0.20 | 0.09 |
| K-562 | 0.56 | 0.79 | 1.14 | 1.19 | 0.13 | 0.17 | 0.21 | 0.15 |
| MOLT-4 | 0.04 | 0.02 | 0.04 | 0.35 | 0.04 | 0.04 | 0.05 | 0.03 |
| RPMI-8226 | 0.87 | 5.26 | 1.47 | 13.90 | 0.15 | 0.20 | 0.14 | 0.05 |
| SR | 0.12 | 0.05 | 0.14 | 0.58 | 0.08 | 0.12 | 0.13 | 0.05 |
| Non-Small Cell | | | | | | | | |
| Lung Cancer | | | | | | | | |
| A549/ATCC | 0.06 | 0.61 | 0.45 | 4.17 | 0.07 | 0.11 | 0.06 | 0.02 |
| EKVX | 1.37 | 2.18 | 1.20 | 7.73 | 0.30 | 0.30 | 0.33 | 0.44 |
| HOP-62 | 0.21 | 0.05 | 0.22 | 0.95 | 0.11 | 0.14 | 0.15 | 0.07 |
| HOP-92 | 1.12 | 2.20 | 1.19 | 4.68 | 0.28 | 0.37 | 0.36 | 0.14 |
| NCI-H226 | 1.04 | 3.27 | 1.35 | 12.30 | 0.15 | 0.16 | 0.14 | 0.15 |
| NCI-H23 | 1.01 | 0.86 | 0.92 | 6.27 | 0.18 | 0.31 | 0.30 | 0.23 |
| NCI-H322M | 1.26 | 1.68 | 1.28 | 4.34 | 0.32 | 0.45 | 0.44 | 0.96 |
| NCI-H460 | 0.04 | 0.02 | 0.05 | 0.38 | 0.04 | 0.05 | 0.05 | 0.03 |
| NCI-H522 | 0.61 | 0.27 | 0.37 | 6.32 | 0.04 | 0.06 | 0.06 | 0.12 |
| Colon Cancer | | | | | | | | |
| COLO 205 | 0.78 | 0.16 | 0.50 | 1.30 | 0.09 | 0.07 | 0.07 | 0.15 |
| HCC-2998 | 1.29 | 5.07 | 1.41 | 11.30 | 0.40 | 1.15 | 1.06 | 1.05 |
| HCT-116 | 0.30 | 0.10 | 0.55 | 0.98 | 0.09 | 0.14 | 0.14 | 0.09 |
| HCT-15 | 0.26 | 1.73 | 1.11 | 4.08 | 0.16 | 0.25 | 0.23 | 0.13 |
| HT29 | 0.66 | 0.16 | 1.08 | 1.37 | 0.07 | 0.15 | 0.15 | 0.15 |
| KM12 | 0.55 | 2.11 | 1.10 | 3.37 | 0.17 | 0.24 | 0.26 | 0.17 |
| SW-620 | 0.09 | 0.03 | 0.08 | 0.30 | 0.12 | 0.18 | 0.17 | 0.11 |
| CNS Cancer | | | | | | | | |
| SF-268 | 1.07 | 0.17 | 0.40 | 4.75 | 0.21 | 0.38 | 0.39 | 0.28 |
| SF-295 | 0.08 | 0.03 | 0.13 | 0.67 | 0.09 | 0.19 | 0.19 | 0.13 |
| SF-539 | 1.33 | 1.98 | 0.73 | 2.15 | 0.25 | 0.37 | 0.32 | 0.53 |
| SNB-19 | 1.06 | 1.03 | 0.61 | 13.80 | 0.18 | 0.22 | 0.16 | 0.13 |
| SNB-75 | 0.29 | 0.35 | 0.26 | 0.93 | 0.22 | 0.25 | 0.36 | 0.37 |
| U251 | 0.31 | 0.04 | 0.30 | 7.24 | 0.08 | 0.11 | 0.10 | 0.08 |
| Melanoma | | | | | | | | |
| LOX IMVI | 0.17 | 0.04 | 0.16 | 0.78 | 0.11 | 0.14 | 0.14 | 0.18 |
| MALME-3M | 1.54 | 5.90 | 1.48 | 5.77 | 0.20 | 0.32 | 0.33 | 1.13 |
| M14 | 1.23 | 0.87 | 0.57 | 1.71 | 0.23 | 0.31 | 0.34 | 0.37 |
| MDA-MB -435 | 1.25 | 5.06 | 1.07 | 1.53 | 0.31 | 0.47 | 0.48 | 0.58 |
| SK-MEL-2 | 2.11 | 7.17 | 13.20 | 5.29 | 0.51 | 1.60 | 1.13 | 1.71 |
| SK-MEL-28 | 1.81 | 8.34 | 1.62 | 2.30 | 0.94 | 1.44 | 1.23 | 1.52 |
| SK-MEL-5 | 1.07 | 2.54 | 1.12 | 11.00 | 0.16 | 0.31 | 0.25 | 0.22 |
| UACC-257 | 2.19 | 7.85 | 2.24 | 4.04 | 0.47 | 0.49 | 0.40 | 1.29 |
| UACC-62 | 1.43 | 2.09 | 0.57 | 1.81 | 0.27 | 0.61 | 0.34 | 1.26 |
| Ovarian Cancer | | | | | | | | |
| IGROV1 | 1.13 | 2.47 | 1.14 | 10.40 | 0.21 | 0.32 | 0.33 | 0.48 |
| OVCAR-3 | 1.43 | 5.97 | 1.41 | 15.10 | 0.19 | 0.26 | 0.28 | 0.34 |
| OVCAR-4 | 1.17 | 1.43 | 1.27 | 2.19 | 0.30 | 0.42 | 0.46 | 0.89 |
| OVCAR-5 | 1.74 | 5.02 | 1.69 | 3.56 | 0.28 | 0.35 | 0.33 | 0.33 |
| OVCAR-8 | 1.10 | 0.81 | 1.18 | 3.09 | 0.14 | 0.19 | 0.18 | 0.13 |
| NCl/ADR-RES | 1.09 | 0.22 | 0.54 | 0.98 | 0.23 | 0.43 | 0.37 | 0.86 |
| SK-OV-3 | 1.16 | 1.47 | 0.66 | 10.10 | 0.22 | 0.23 | 0.22 | 0.21 |
| Renal Cancer | | | | | | | | |
| 786-0 | 0.47 | 1.47 | 0.32 | 2.47 | 0.12 | 0.20 | 0.20 | 0.13 |
| A498 | 0.55 | 0.38 | 0.53 | 0.96 | 0.20 | 0.37 | 0.25 | 0.35 |
| ACHN | 0.18 | 0.08 | 0.22 | 0.78 | 0.06 | 0.12 | 0.13 | 0.06 |
| CAKI-1 | 0.41 | 0.03 | 0.06 | 2.97 | ND | ND | ND | ND |
| RXF 393 | 1.04 | 0.70 | 1.30 | 1.90 | 0.23 | 0.32 | 0.26 | 0.27 |
| SN12C | 0.49 | 2.05 | 1.18 | 10.20 | 0.16 | 0.21 | 0.15 | 0.08 |
| TK-10 | ND^{e} | ND | ND | ND | 0.18 | 0.33 | 0.30 | 0.27 |
| U0-31 | 1.03 | 0.60 | 1.08 | 3.74 | 0.08 | 0.13 | 0.12 | 0.13 |
| Prostate Cancer | | | | | | | | |
| PC-3 | 0.98 | 0.86 | 1.02 | 3.60 | 0.28 | 0.37 | 0.30 | 0.60 |
| DU-145 | 0.24 | 0.19 | 0.33 | 3.18 | 0.21 | 0.20 | 0.22 | 0.06 |
| Breast Cancer | | | | | | | | |
| MCF7 | 0.09 | 0.02 | 0.05 | 0.49 | 0.03 | 0.03 | 0.04 | 0.03 |
| MDA-MB- | | | | | | | | |
| 231/ATCC | 1.35 | 6.15 | 1.62 | 10.70 | 0.40 | 0.70 | 0.40 | 0.48 |
| HS 578T | 1.49 | 6.87 | 1.84 | 12.90 | 0.75 | 2.24 | 1.62 | 2.08 |
| BT-549 | 1.53 | 3.80 | 0.65 | 15.20 | 0.66 | 0.58 | 0.94 | 1.25 |
| T-47D | 8.98 | 1.21 | 1.12 | 22.80 | 0.08 | 0.16 | 0.15 | 0.27 |
| MDA-MIB-468 | 0.55 | 0.06 | 1.28 | 1.19 | 0.23 | 0.22 | 0.14 | 0.21 |
| Compound No. | 9 | 10 | 11 | 12 | 13 | 19 | 20 | |
| MYC Inhibition^{a} | ++++ | ++++ | ++++ | ++++ | +++ | ++ | + | |
| Topl Inhibiton^{b} | ++ | +++ | +++ | ++++ | ++++ | +++ | +++ | |
| MGM (uM)^{c} | 0.045 | 0.074 | 0.177 | 0.055 | 0.140 | 2.570 | 1.260 | |
| Cancer Cell Lines | Antiproliferative Activities [GI50 (μM)]^{d} | | | | | | | |
| Leukemia | | | | | | | | |
| CCRF-CEM | <0.01 | 0.04 | 0.05 | <0.01 | 0.01 | 1.57 | 1.37 | |
| HL-60(TB) | 0.03 | 0.04 | 0.10 | <0.01 | 0.21 | 4.28 | 1.80 | |
| K-562 | 0.02 | 0.04 | 0.23 | 0.11 | 0.18 | 5.13 | 1.42 | |
| MOLT-4 | <0.01 | 0.03 | 0.03 | <0.01 | <0.01 | 0.87 | 0.29 | |
| RPMI-8226 | 0.04 | 0.08 | 0.17 | 0.03 | 0.10 | 1.85 | 1.84 | |
| SR | <0.01 | 0.01 | 0.02 | <0.01 | <0.01 | 0.29 | 0.31 | |
| Non-Small Cell | | | | | | | | |
| Lung Cancer | | | | | | | | |
| A549/ATCC | 0.05 | 0.07 | 0.12 | 0.04 | 0.06 | 1.96 | 0.59 | |
| EKVX | 0.30 | 0.27 | 0.45 | 1.01 | 0.43 | 4.91 | ND | |
| HOP-62 | <0.01 | 0.03 | 0.15 | 0.01 | 0.03 | 1.41 | 1.25 | |
| HOP-92 | 0.55 | 0.24 | 0.87 | 1.38 | 0.15 | 4.47 | 1.42 | |
| NCI-H226 | 0.10 | 0.09 | 0.17 | 0.04 | 1.42 | 2.79 | 1.32 | |
| NCI-H23 | 0.03 | 0.04 | 0.15 | 0.02 | 0.21 | 3.10 | 1.86 | |
| NCI-H322M | 0.05 | 0.16 | 0.28 | 0.04 | 0.34 | 5.44 | 2.47 | |
| NCI-H460 | <0.01 | 0.02 | 0.04 | <0.01 | <0.01 | 0.44 | 0.41 | |
| NCI-H522 | 0.06 | 0.08 | 0.08 | <0.01 | 0.02 | 1.06 | 1.84 | |
| Colon Cancer | | | | | | | | |
| COLO 205 | 0.04 | 0.06 | 0.30 | 0.24 | 0.07 | 1.74 | 0.47 | |
| HCC-2998 | 0.16 | 0.20 | 0.30 | 1.03 | 0.19 | 3.44 | 2.17 | |
| HCT-116 | <0.01 | 0.03 | ND | ND | 0.04 | 1.27 | 0.44 | |
| HCT-15 | 0.06 | 0.12 | 0.30 | 1.17 | 0.15 | 2.90 | 1.03 | |
| HT29 | 0.02 | 0.06 | 0.15 | 0.03 | 0.12 | 2.48 | 1.01 | |
| KM12 | 0.14 | 0.16 | 0.40 | 1.11 | 0.19 | 3.80 | 1.61 | |
| SW-620 | <0.01 | 0.03 | 0.13 | 0.01 | 0.04 | 1.68 | 0.46 | |
| CNS Cancer | | | | | | | | |
| SF-268 | 0.02 | 0.07 | 0.08 | <0.01 | 0.07 | 1.75 | 2.15 | |
| SF-295 | <0.01 | 0.03 | 0.07 | 0.01 | 0.08 | 1.27 | 0.75 | |
| SF-539 | 0.03 | 0.05 | 0.31 | 0.04 | 0.11 | 1.96 | 0.85 | |
| SNB-19 | 0.04 | 0.04 | 0.11 | 0.01 | 0.11 | 2.13 | 1.75 | |
| SNB-75 | 0.05 | 0.13 | 0.27 | 0.03 | 0.23 | 3.63 | 0.66 | |
| U251 | 0.01 | 0.04 | 0.08 | <0.01 | 0.10 | 1.72 | 1.05 | |
| Melanoma | | | | | | | | |
| LOX IMVI | <0.01 | 0.03 | 0.04 | <0.01 | 0.05 | 1.34 | 0.81 | |
| MALME-3M | 0.06 | 0.18 | 0.34 | 0.62 | 0.21 | 4.49 | ND | |
| M14 | <0.01 | 0.04 | 0.07 | <0.01 | 0.32 | 2.85 | 1.57 | |
| MDA-MB-435 | 0.06 | 0.15 | 0.42 | 0.48 | 0.39 | 4.67 | 1.93 | |
| SK-MEL-2 | 1.01 | 0.35 | 1.59 | 1.56 | 0.55 | 19.50 | 3.92 | |
| SK-MEL-28 | 10.60 | 0.39 | 1.13 | 1.18 | 1.11 | 8.03 | 1.56 | |
| SK-MEL-5 | 0.07 | 0.11 | 0.26 | 0.03 | 0.07 | 1.50 | 1.00 | |
| UACC-257 | 0.18 | 0.18 | 0.59 | 0.58 | 1.18 | 6.60 | 2.02 | |
| UACC-62 | 0.02 | 0.02 | 0.04 | <0.01 | 0.55 | 1.58 | 1.30 | |
| Ovarian Cancer | | | | | | | | |
| IGROV1 | 0.02 | 0.21 | 0.42 | 0.08 | ND | ND | 1.91 | |
| OVCAR-3 | 0.06 | 0.17 | 0.32 | 0.14 | 0.41 | 2.70 | 2.41 | |
| OVCAR-4 | 0.08 | 0.17 | 0.33 | 0.48 | 0.23 | 3.13 | 1.93 | |
| OVCAR-5 | 0.16 | 0.14 | 0.45 | 1.04 | 0.38 | 6.31 | 2.44 | |
| OVCAR-8 | 0.09 | 0.09 | 0.17 | 0.02 | 0.07 | 3.11 | 0.95 | |
| NCl/ADR-RES | 0.03 | 0.06 | 0.13 | 0.02 | 1.04 | 3.14 | 2.72 | |
| SK-OV-3 | 0.03 | 0.06 | 0.14 | 0.01 | 0.22 | 2.67 | 2.22 | |
| Renal Cancer | | | | | | | | |
| 786-0 | 0.03 | 0.04 | 0.22 | 0.02 | 0.06 | 1.78 | 1.16 | |
| A498 | ND | 0.03 | ND | ND | 0.13 | 1.64 | ND | |
| ACHN | <0.01 | 0.04 | 0.04 | <0.01 | 0.04 | 1.67 | 0.29 | |
| CAKI-1 | <0.01 | 0.03 | 0.04 | <0.01 | 0.24 | 3.17 | 0.57 | |
| RXF 393 | 0.06 | 0.07 | 0.39 | 0.03 | 0.96 | 7.16 | 2.31 | |
| SN12C | 0.04 | 0.05 | 0.08 | <0.01 | 0.16 | 4.10 | 1.12 | |
| TK-10 | 0.37 | 0.32 | 0.73 | 1.34 | 0.41 | 4.63 | 3.02 | |
| U0-31 | <0.01 | 0.03 | 0.05 | <0.01 | 0.10 | 0.42 | 0.57 | |
| Prostate Cancer | | | | | | | | |
| PC-3 | 0.07 | 0.13 | 0.57 | 1.28 | 0.28 | 19.50 | 2.05 | |
| DU-145 | 0.02 | 0.05 | 0.06 | <0.01 | 0.06 | 2.31 | 1.89 | |
| Breast Cancer | | | | | | | | |
| MCF7 | <0.01 | 0.01 | 0.03 | <0.01 | <0.01 | 0.20 | 0.37 | |
| MDA-MB- | | | | | | | | |
| 231/ATCC | 0.68 | 0.27 | 0.63 | 1.61 | 0.68 | 11.20 | 2.58 | |
| HS 578T | 1.91 | 1.17 | 3.85 | 1.82 | 0.66 | 8.95 | 3.08 | |
| BT-549 | 0.35 | 0.37 | 0.29 | 0.05 | 0.28 | 4.11 | 1.67 | |
| T-47D | 0.02 | 0.03 | 0.11 | <0.01 | 0.05 | 1.07 | 2.04 | |
| MDA-MIB-468 | 0.03 | 0.03 | 0.12 | 0.03 | ND | ND | 1.57 | |
| Compound No. | 25 | 30 | 36 | 37 | 38 | 42 | | |
| MYC Inhibition | 0 | + | ++++ | +++ | + | +++ | | |
| Topl Inhibiton | ++ | 0 | ++ | ++ | ++ | +++++ | | |
| MGM (uM) | 0.720 | 0.600 | 0.280 | 1.860 | 0.660 | 0.570 | | |
| Cancer Cell Lines | Antiproliferative Activities [GI50 (μM)]^{d} | | | | | | | |
| Leukemia | | | | | | | | |
| CCRF-CEM | 0.34 | 0.03 | 0.05 | 0.66 | 0.11 | <0.01 | | |
| HL-60(TB) | 1.17 | 0.03 | 0.08 | 0.59 | 1.48 | 0.02 | | |
| K-562 | 0.83 | 0.34 | 0.09 | 2.25 | 0.77 | 1.49 | | |
| MOLT-4 | 0.10 | 0.02 | 0.03 | 0.24 | 0.15 | <0.01 | | |
| RPMI-8226 | 0.27 | 0.12 | 0.12 | 0.70 | 1.02 | <0.01 | | |
| SR | 0.31 | 0.02 | 0.03 | 0.03 | 0.04 | <0.01 | | |
| Non-Small Cell | | | | | | | | |
| Lung Cancer | | | | | | | | |
| A549/ATCC | 0.22 | 0.06 | 0.52 | ND | 1.31 | 10.50 | | |
| EKVX | 1.18 | 2.91 | ND | ND | ND | 70.70 | | |
| HOP-62 | 1.06 | 0.18 | 0.12 | 0.59 | 0.46 | <0.01 | | |
| HOP-92 | 1.72 | 0.13 | 0.28 | 13.60 | 1.34 | 0.19 | | |
| NCI-H226 | 0.56 | 0.27 | 0.45 | ND | 1.18 | 0.37 | | |
| NCI-H23 | 1.05 | 0.24 | 0.17 | 0.68 | 0.38 | 0.09 | | |
| NCI-H322M | 0.45 | 7.05 | 0.66 | 72.60 | 1.39 | >100 | | |
| NCI-H460 | 0.31 | 0.04 | 0.03 | 0.24 | 0.37 | <0.01 | | |
| NCI-H522 | 0.96 | 0.84 | 0.05 | 0.10 | 0.78 | <0.01 | | |
| Colon Cancer | | | | | | | | |
| COLO 205 | 0.62 | 4.92 | 7.76 | 7.90 | 0.69 | >100 | | |
| HCC-2998 | 0.35 | 10.00 | 1.42 | 37.10 | 0.62 | 29.30 | | |
| HCT-116 | ND | 0.39 | 0.22 | 0.74 | 0.15 | 0.17 | | |
| HCT-15 | 0.73 | 0.09 | 1.53 | >100 | 1.49 | >100 | | |
| HT29 | 0.35 | 0.34 | 1.18 | 4.08 | 0.48 | >100 | | |
| KM12 | 1.13 | 0.53 | 1.04 | 9.39 | 1.34 | >100 | | |
| SW-620 | 0.16 | 0.20 | 0.08 | 0.81 | 0.34 | 0.06 | | |
| CNS Cancer | | | | | | | | |
| SF-268 | 0.95 | 1.85 | 0.04 | 0.27 | 0.55 | ND | | |
| SF-295 | 1.16 | 0.36 | 0.18 | 0.33 | 0.28 | <0.01 | | |
| SF-539 | 1.61 | 1.04 | 0.27 | 0.37 | 0.67 | <0.01 | | |
| SNB-19 | 0.54 | 0.33 | 0.39 | 12.80 | 1.08 | <0.01 | | |
| SNB-75 | 0.95 | 1.39 | 0.21 | 2.97 | 0.16 | 0.23 | | |
| U251 | 0.64 | 0.18 | 0.12 | 0.27 | 0.33 | <0.01 | | |
| Melanoma | | | | | | | | |
| LOX IMVI | 0.15 | 0.10 | 0.09 | 0.30 | 0.30 | 0.06 | | |
| MALME-3M | 1.42 | 0.93 | 1.16 | 1.79 | 2.16 | 1.60 | | |
| M14 | 1.05 | 1.65 | 0.08 | ND | 0.46 | <0.01 | | |
| MDA-MB-435 | 1.20 | 10.30 | 0.89 | 2.84 | 1.30 | >100 | | |
| SK-MEL-2 | 1.71 | 18.20 | 9.44 | 30.80 | 2.18 | >100 | | |
| SK-MEL-28 | 1.53 | 2.01 | 1.37 | 16.40 | 1.88 | >100 | | |
| SK-MEL-5 | 0.70 | 0.48 | 0.22 | 1.67 | 1.26 | 0.22 | | |
| UACC-257 | 1.69 | 12.30 | 0.48 | 25.30 | 1.57 | >100 | | |
| UACC-62 | 1.03 | 0.72 | 0.05 | 0.21 | 0.95 | <0.01 | | |
| Ovarian Cancer | | | | | | | | |
| IGROV1 | 0.84 | 0.58 | 1.52 | 11.30 | 1.24 | 6.72 | | |
| OVCAR-3 | 1.05 | 6.09 | 0.57 | 1.35 | 1.69 | 16.40 | | |
| OVCAR-4 | 0.34 | 1.45 | 1.29 | 1.41 | 1.11 | >100 | | |
| OVCAR-5 | 2.12 | 1.51 | 3.60 | >100 | 1.67 | >100 | | |
| OVCAR-8 | 0.40 | 0.16 | 0.45 | 0.89 | 1.01 | 1.03 | | |
| NCl/ADR-RES | 1.16 | 1.48 | 0.28 | >100 | 2.55 | 0.35 | | |
| SK-OV-3 | 1.05 | 1.21 | 0.24 | 0.56 | 1.15 | <0.01 | | |
| Renal Cancer | | | | | | | | |
| 786-0 | 1.34 | 0.22 | 0.21 | 0.33 | 0.34 | <0.01 | | |
| A498 | ND | 10.60 | 0.07 | ND | 0.96 | 7.17 | | |
| ACHN | 0.39 | 0.09 | 0.05 | 0.31 | 0.28 | <0.01 | | |
| CAKI-1 | 1.05 | ND | 0.12 | 0.22 | 0.29 | 0.32 | | |
| RXF 393 | 1.29 | 1.66 | 0.46 | 1.35 | 1.13 | 8.00 | | |
| SN12C | 0.33 | 0.25 | 0.20 | 0.42 | 0.45 | <0.01 | | |
| TK-10 | 1.33 | 2.75 | 2.12 | 2.54 | 1.85 | 73.70 | | |
| U0-31 | 0.77 | 0.30 | 0.07 | ND | 0.69 | 0.03 | | |
| Prostate Cancer | | | | | | | | |
| PC-3 | 0.86 | 1.21 | 0.67 | 7.22 | 0.64 | 34.90 | | |
| DU-145 | 0.33 | 0.20 | 0.14 | 0.33 | 0.43 | <0.01 | | |
| Breast Cancer | | | | | | | | |
| MCF7 | 0.30 | 0.04 | 0.05 | 0.08 | 0.16 | <0.01 | | |
| MDA-MB- | | | | | | | | |
| 231/ATCC | 0.51 | 1.27 | 2.06 | 18.10 | 1.57 | 77.80 | | |
| HS 578T | 1.44 | 4.90 | 1.97 | >100 | ND | 54.20 | | |
| BT-549 | 1.27 | 3.10 | 0.12 | 0.35 | 0.36 | ND | | |
| T-47D | 1.55 | 0.63 | 0.56 | 12.00 | 0.70 | <0.01 | | |
| MDA-MB-468 | 0.95 | 7.84 | 0.14 | ND | 0.24 | ND | | |
| No. in JACS | 44 | 45 | 46 | 47 | 48 | 49 | 50 | 55 |
| MYC Inhibition | +++ | ++ | + | ++ | + | + | ++ | + |
| Topl Inhibiton | ++++ | +++ | +++ | ++++ | + | + | ++++ | ++ |
| MGM (uM) | 0.060 | 0.180 | 0.350 | 0.390 | 1.020 | 12.000 | 0.040 | 0.340 |
| Cancer Cell Lines | Antiproliferative Activities [GI₅₀ (μM]^{d} | | | | | | | |
| Leukemia | | | | | | | | |
| CCRF-CEM | <0.01 | <0.01 | <0.01 | <0.01 | 0.30 | 2.44 | <0.01 | <0.01 |
| HL-60(TB) | <0.01 | 0.22 | 0.25 | 1.60 | >50 | 19.50 | 0.29 | 0.30 |
| K-562 | 0.02 | 0.09 | 0.28 | ND | 1.59 | 6.49 | <0.01 | 0.19 |
| MOLT-4 | <0.01 | <0.01 | <0.01 | <0.01 | 0.42 | <0.01 | <0.01 | <0.01 |
| RPMI-8226 | 0.01 | <0.01 | 0.48 | 0.30 | 0.20 | 2.39 | 0.02 | 0.25 |
| SR | <0.01 | <0.01 | ND | <0.01 | ND | ND | <0.01 | <0.01 |
| Non-Small Cell | | | | | | | | |
| Lung Cancer | | | | | | | | |
| A549/ATCC | 0.03 | 0.11 | 0.74 | 0.89 | 0.65 | 18.70 | <0.01 | 0.11 |
| EKVX | 0.26 | ND | 2.06 | 1.46 | 1.31 | 17.70 | 2.57 | ND |
| HOP-62 | 0.01 | 0.06 | 0.05 | ND | 0.42 | 18.00 | <0.01 | 0.19 |
| HOP-92 | 0.83 | 1.65 | 3.27 | 1.30 | ND | 19.80 | <0.01 | 1.66 |
| NCI-H226 | 0.21 | 1.39 | 0.65 | 1.40 | 0.66 | 13.90 | 0.05 | 2.12 |
| NCI-H23 | <0.01 | 0.13 | 0.05 | 0.19 | 0.71 | 11.90 | 0.02 | 0.71 |
| NCI-H322M | 0.05 | ND | 0.53 | 1.56 | 1.05 | 15.60 | 3.18 | 1.00 |
| NCI-H460 | <0.01 | 0.02 | 0.43 | 0.28 | 0.24 | 5.81 | <0.01 | 0.03 |
| NCI-H522 | <0.01 | 0.07 | 0.40 | 0.31 | 13.10 | 15.40 | 0.02 | 0.04 |
| Colon Cancer | | | | | | | | |
| COLO 205 | 0.38 | 0.30 | 0.08 | 0.94 | 1.14 | 10.30 | <0.01 | 0.31 |
| HCC-2998 | ND | 0.10 | ND | 0.13 | 0.14 | <0.01 | <0.01 | 3.32 |
| HCT-116 | 0.07 | 0.12 | 0.13 | 0.22 | 0.12 | 1.49 | <0.01 | 0.16 |
| HCT-15 | 0.55 | 0.14 | 0.91 | 0.20 | 1.11 | 16.00 | 0.07 | 1.02 |
| HT29 | <0.01 | 0.09 | ND | 0.07 | 1.04 | 16.90 | <0.01 | 0.32 |
| KM12 | 0.26 | 0.15 | 1.00 | 1.29 | 0.68 | 12.80 | 1.62 | 1.01 |
| SW-620 | 0.11 | 0.13 | 0.25 | <0.01 | 0.57 | 11.20 | <0.01 | 0.11 |
| CNS Cancer | | | | | | | | |
| SF-268 | 0.11 | 1.20 | 0.38 | 1.11 | 2.41 | 11.90 | <0.01 | 0.07 |
| SF-295 | <0.01 | <0.01 | 0.87 | 0.02 | 0.57 | 10.70 | <0.01 | 0.03 |
| SF-539 | ND | 0.01 | 0.25 | 0.16 | 0.64 | 17.70 | <0.01 | 0.24 |
| SNB-19 | ND | ND | 0.02 | 0.55 | 1.39 | >100 | <0.01 | 0.31 |
| SNB-75 | 0.01 | 0.13 | ND | 0.79 | 1.30 | 16.00 | <0.01 | 0.40 |
| U251 | <0.01 | 0.03 | 0.01 | 0.06 | 0.52 | 11.40 | <0.01 | 0.05 |
| Melanoma | | | | | | | | |
| LOX IMVI | <0.01 | 0.11 | 0.03 | 0.07 | 0.46 | 3.81 | <0.01 | 0.05 |
| MALME-3M | 0.15 | 1.01 | 0.69 | 1.07 | ND | 24.80 | ND | 1.25 |
| M14 | 0.01 | 0.32 | 0.37 | 1.10 | 1.34 | ND | <0.01 | 0.15 |
| MDA-MB-435 | 0.53 | 1.23 | 0.36 | 0.65 | 1.81 | >100 | <0.01 | 0.95 |
| SK-MEL-2 | 1.62 | 1.93 | 3.39 | 4.99 | 3.01 | >100 | 19.60 | 5.32 |
| SK-MEL-28 | 0.17 | 1.27 | 0.25 | 1.48 | 1.42 | 16.50 | 0.68 | 11.80 |
| SK-MEL-5 | 0.14 | 1.33 | 0.48 | 0.33 | 2.26 | 14.20 | 0.21 | 1.15 |
| UACC-257 | 0.29 | 1.54 | 0.95 | 1.34 | 7.72 | >100 | 0.12 | 0.70 |
| UACC-62 | 0.03 | 0.36 | 0.27 | 0.54 | 1.38 | 15.10 | 0.01 | 0.33 |
| Ovarian Cancer | | | | | | | | |
| IGROV1 | 0.27 | 0.32 | 2.57 | 1.04 | 18.90 | 7.05 | 0.02 | 0.11 |
| OVCAR-3 | 0.58 | 0.43 | 0.10 | 1.19 | 0.71 | 15.30 | 0.04 | 1.23 |
| OVCAR-4 | 0.12 | 0.14 | 0.10 | 0.33 | 0.25 | 4.17 | 1.48 | 2.21 |
| OVCAR-5 | 0.15 | 0.25 | 0.79 | ND | 1.53 | 17.60 | 0.13 | 12.80 |
| OVCAR-8 | 0.11 | 0.14 | 0.28 | 0.48 | 0.59 | 18.10 | <0.01 | 0.34 |
| NCl/ADR-RES | ND | ND | 8.70 | 0.03 | 2.44 | 1.55 | <0.01 | 1.50 |
| SK-OV-3 | 0.60 | 1.40 | ND | 1.29 | 2.18 | 12.30 | <0.01 | 0.21 |
| Renal Cancer | | | | | | | | |
| 786-0 | <0.01 | 0.11 | 0.09 | 0.17 | 0.76 | 12.90 | <0.01 | 0.29 |
| A498 | 0.38 | 1.13 | ND | 20.20 | 5.94 | ND | 1.52 | ND |
| ACHN | <0.01 | 0.09 | 0.11 | 0.15 | 0.64 | 10.10 | <0.01 | 0.07 |
| CAKI-1 | <0.01 | 0.11 | 0.29 | 0.20 | 0.38 | 10.30 | <0.01 | 0.10 |
| RXF 393 | 0.14 | 0.62 | 2.97 | 2.48 | 1.30 | 20.50 | 0.05 | 0.80 |
| SN12C | <0.01 | 0.59 | 0.26 | 0.94 | 0.44 | 11.40 | <0.01 | 0.24 |
| TK-10 | ND | ND | 2.95 | 1.66 | 0.52 | 17.10 | 12.50 | ND |
| U0-31 | 0.02 | 0.29 | 16.90 | 1.05 | 3.75 | 20.60 | 0.15 | 0.32 |
| Prostate Cancer | | | | | | | | |
| PC-3 | ND | ND | 1.15 | ND | 1.13 | 15.80 | 0.50 | 0.62 |
| DU-145 | <0.01 | 0.05 | 0.25 | 0.16 | 0.11 | 10.60 | <0.01 | 0.04 |
| Breast Cancer | | | | | | | | |
| MCF7 | <0.01 | <0.01 | 0.36 | 0.09 | 0.38 | 8.82 | <0.01 | 0.07 |
| MDA-MB- | | | | | | | | |
| 231/ATCC | 0.46 | 0.25 | 0.64 | 0.97 | 0.68 | 15.30 | 0.15 | 1.59 |
| HS 578T | 2.03 | 2.29 | 0.55 | 1.93 | 2.85 | 13.90 | 1.52 | ND |
| BT-549 | 0.75 | 1.30 | 0.88 | ND | 2.41 | 80.80 | 2.40 | 4.70 |
| T-47D | 0.02 | 0.12 | 0.07 | 0.50 | 0.73 | ND | <0.01 | 5.51 |
| MDA-MB-468 | ND | ND | ND | ND | ND | ND | ND | 0.55 |

^{a}The MYC inhibition levels were determined based on the western blotting results as shown in
FIGS 3A and 12A to 12F. MYC inhibition levels were classified into four levels: strong
inhibition,
+++, MYC expression inhibited at 0.5 to 1.0 μM;
medium inhibition,
++, MYC expression inhibited at 2.0 μM, or no clear dose-dependent MYC inhibition; weak inhibition,
+, MYC expression inhibited at 4.0 μM; no inhibition, 0, no MYC expression inhibition up to 4.0 μM.
^{b}The relative topoisomerase I (Top 1) inhibition levels of the compounds were previously
determined and classified into six levels (0 - 5, +++++ = 5).^{3, 6-9, 15, 50-52}
^{c}The MGM values for each compound are the average of GI₅₀ values across the entire panel of
NCI-60 cancer cell lines, where compounds with GI₅₀ values that fall outside the test range of
10⁻⁴ to 10⁻⁸ M are assigned values of 10⁻⁴ or 10⁻⁸M.
^{d}The antiproliferative activities [GI₅₀ values, μM)] listed are the concentrations corresponding to
50% growth inhibition which were determined in the NCI-60 cancer cell lines drug screen.
^{e}GI₅₀ value not determined.

TABLE 4
Raw log₁₀MGM data of the 29 indenoisoquinolines.

| | | MYC Inhibition Levels* | | | |
|---|---|---|---|---|---|
| log₁₀MGM (μM) | | 3 | 2 | 1 | 0 |
| Topoisomerase I | 0 | | | −0.22 | |
| Inhibition | | | | Mean: −0.22 | |
| Levels** | 1 | −0.62*** | 0.46 | −0.66, −0.24, −0.12, 1.08 | |
| | | Mean: −0.62**** | Mean: 0.46 | Mean: 0.02 | |
| | 2 | −1.35, −0.78, −0.70, −0.66, | | −1.18, −0.22 | −0.10 |
| | | −0.22, −0.20, −0.13 | | Mean: −0.70 | Mean: −0.10 |
| | | Mean: −0.58 | | | |
| | 3 | −1.13, −0.75 | | −0.80, −0.06, 0.10, 0.43 | |
| | | Mean: −0.94 | | Mean: −0.08 | |
| | 4 | −1.26, −0.40 | −0.95, −0.68, −0.46 | | |
| | | Mean: −0.83 | Mean: −0.69 | | |
| | 5 | | −1.10 | | |
| | | | Mean: −1.10 | | |

The 29 indenoisoquinolines were grouped by their MYC inhibition levels and topoisomerase I inhibition levels. The overall anticancer activity of each group was determined by the mean(log₁₀MGM) value.
*The MYC inhibition levels were determined based on the western blotting results as shown in FIGs 3A and 12A to 12F (3 = strong, 2 = medium, 1 = weak, and 0 = no inhibition).
**The topoisomerase I inhibition levels were previously determined.^{3, 6-9, 15, 50-52}
***log₁₀MGM value of each individual compound. The MGM values for each compound are the average of GI₅₀ values across the entire panel of NCI-60 cancer cell lines, where compounds with GI₅₀ values that fall outside the test range of 10⁻⁴ to 10⁻⁸ M are assigned values of 10⁻⁴ or 10⁻⁸ M. 50% growth inhibition (GI₅₀) values were determined in the NCI-60 cancer cell lines drug screen.
**** the mean(log₁₀MGM) value of all compounds in each group.

### MYC-Inhibiting Indenoisoquinolines are Strong MYC G4 Binding Ligands

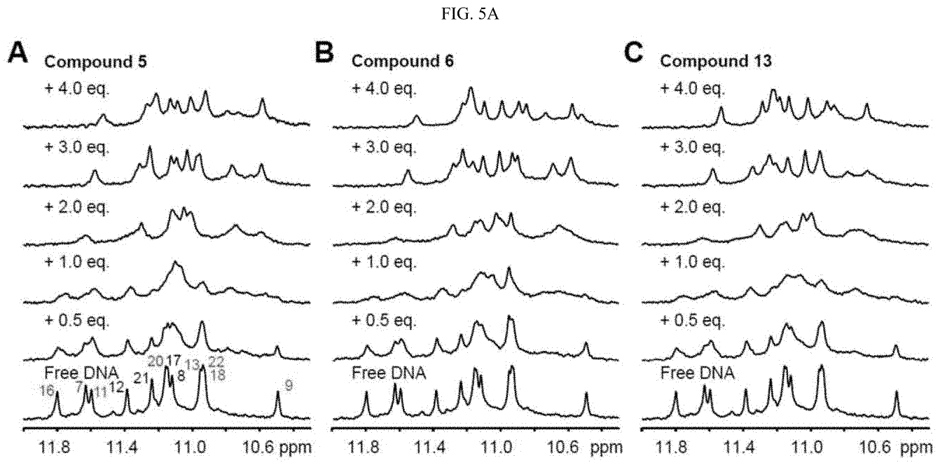

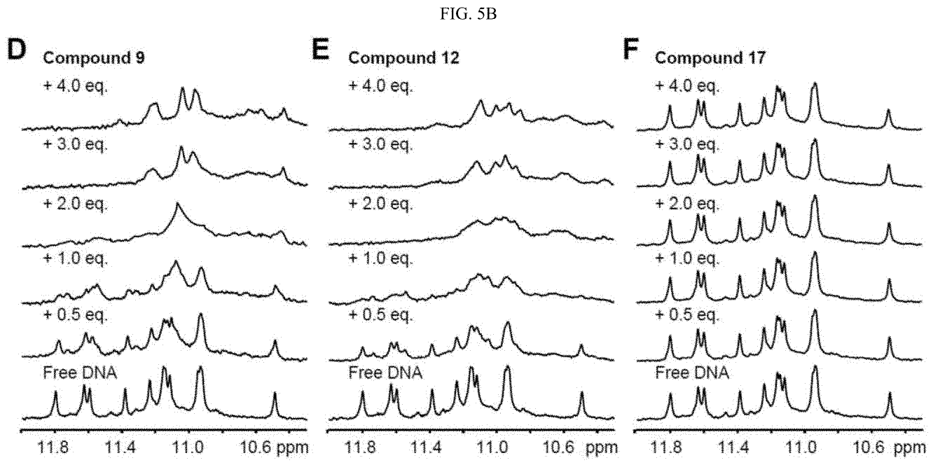

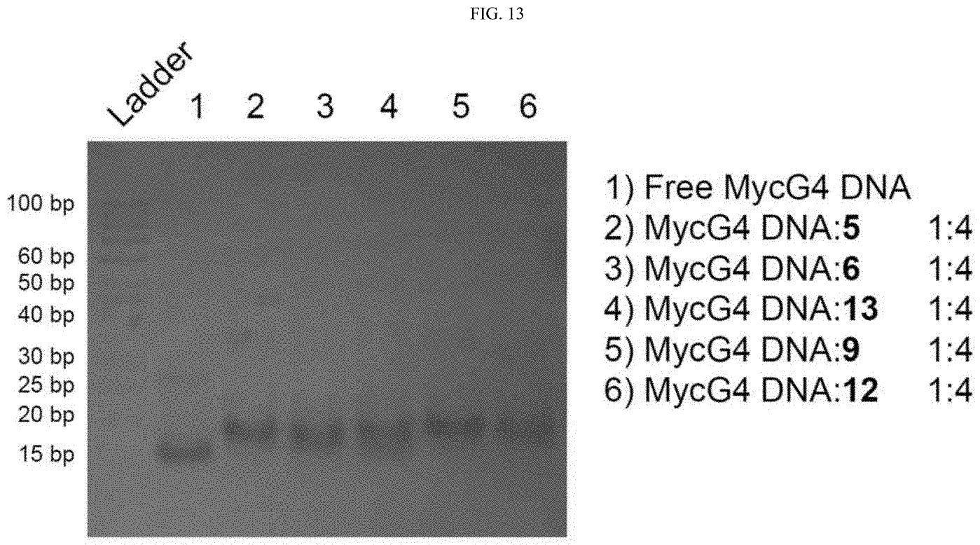

The binding interactions of six selected indenoisoquinolines with MycG4 were examined using ¹H NMR titration experiments in K⁺-containing solution. The free MycG4 DNA shows 12 imino proton peaks of guanines from the three G-tetrads (FIG. 5).^{43, 48} Upon respective addition of the five MycG4-interactive indenoisoquinolines, clear changes of the tetrad-guanine imino proton signals were observed, confirming the binding of these compounds to MycG4 (FIG. 5A-E). The binding appeared to be in the medium-to-fast exchange rate on the NMR time-scale, as shown by the broadening of DNA proton peaks at lower drug equivalence (0.5 and 1) and the sharpening at higher drug equivalence (2 and 3). Indenoisoquinolines appeared to bind at both ends of the MycG4, as shown by the imino proton peaks corresponding to both of the 3′- and 5′-tetrads being significantly shifted upon drug addition. Three MYC-inhibiting compounds, the 7-azaindenoisoquinolines 5 and 6, and the indenoisoquinoline 13, showed more specific binding to MycG4, where a well-defined complex was shown to form at the drug equivalence of 3, with a new set of 12 imino proton peaks. For compound binding at intermediate exchange rate on the NMR time scale, a compound:DNA ratio higher than its binding stoichiometry is needed to push the equilibrium towards the formation of a stable drug-DNA complex, as shown by the sharp, well-resolved proton peaks.^{48, 56} In contrast, the negative control compound 17 did not show any binding as no change was observed in the ¹H NMR spectra upon titration (FIG. 5F). The MycG4 complexes of the five MycG4-interactive indenoisoquinolines were monomeric in nature as shown by native EMSA gels (FIG. 13).

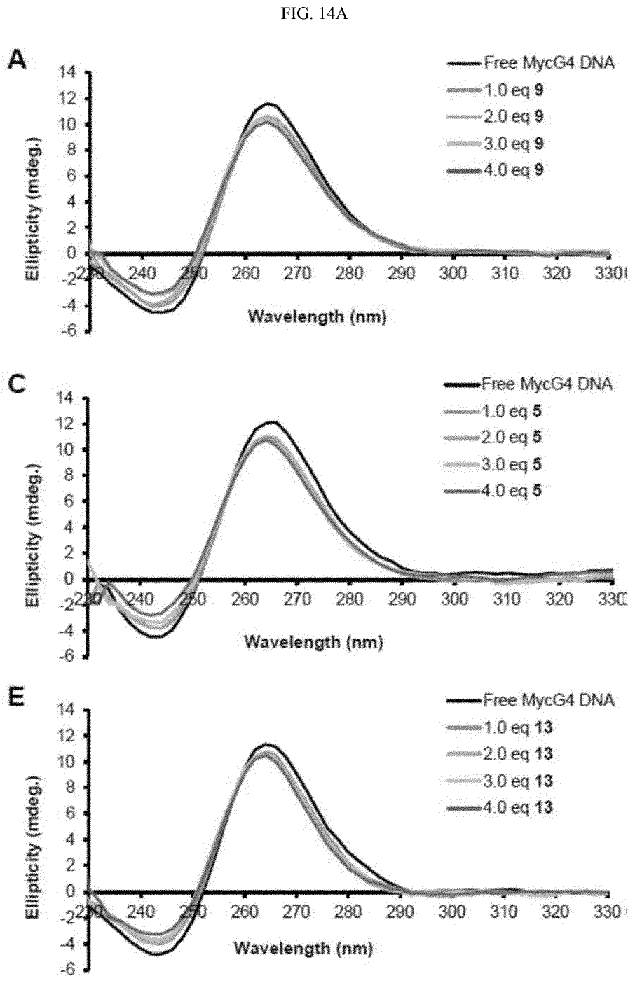

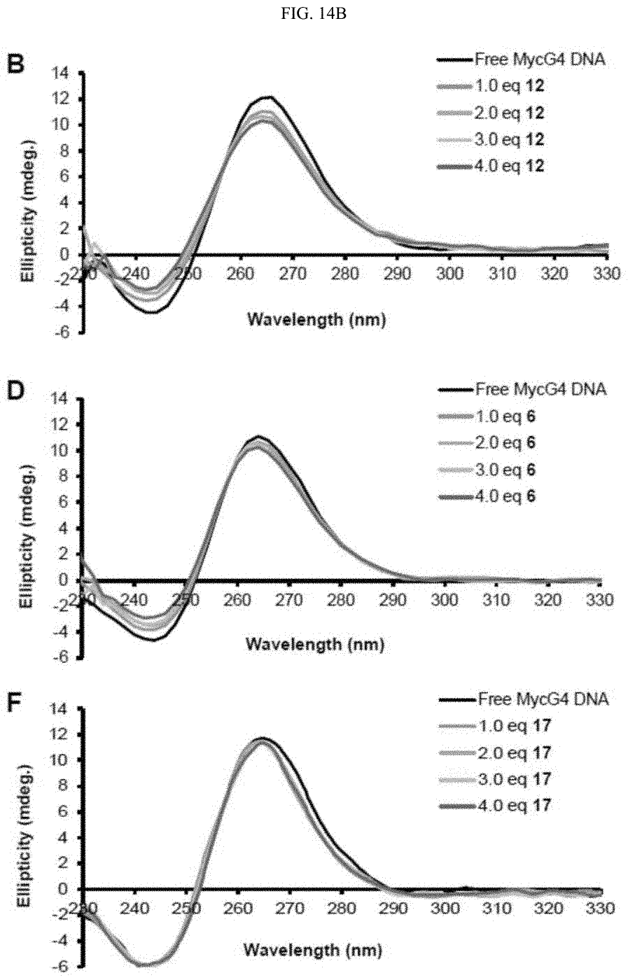

CD titration experiments with MycG4 were also carried out for the six selected indenoisoquinolines. The free MycPu22 DNA in K⁺ buffer showed the CD signature of a parallel G-quadruplex, with a positive peak at 264 nm and a negative peak at 242 nm.⁵⁷ Upon addition of indenoisoquinolines, the CD signature of a parallel G-quadruplex was maintained (FIG. 14). The five MycG4-interactive compounds showed a slight decrease in intensity for both the positive peak at 264 nm and the negative peak at 242 nm, likely due to the ligand-induced capping structure formation by the flanking segments. The decrease in intensity in CD spectra of G4 upon ligand binding has been previously reported.⁵⁸ The negative control compound 17 showed no effect on the CD spectrum.

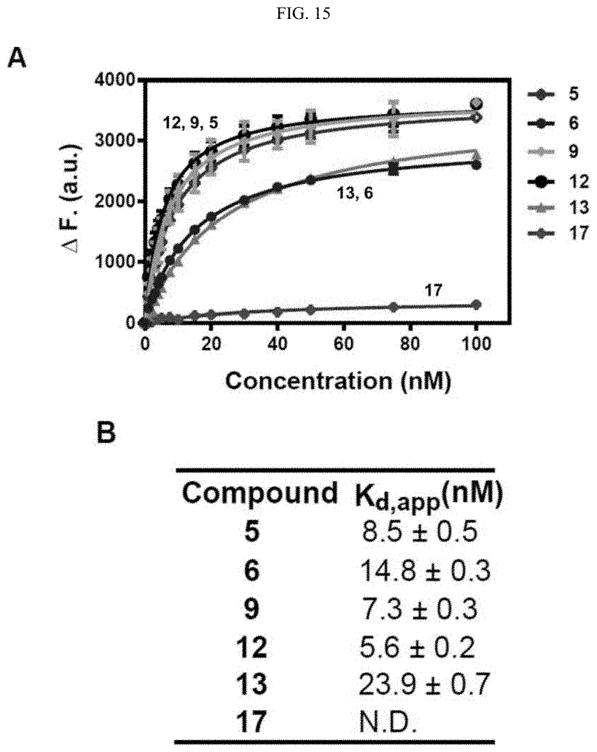

Binding affinities of these six indenoisoquinolines to MycG4 were measured using a 3′-TAMRA-labeled MycPu22 DNA.⁵⁹ The five MYC-inhibiting compounds showed strong binding with apparent binding affinity K_{d} values of 5.6-23.9 nM, whereas the negative control compound showed negligible binding (FIG. 15). The indenoisoquinolines show negligible fluorescence in either the free or bound state.

### Molecular Docking Study of the Binding of Indenoisoquinoline 5 to MYC G4

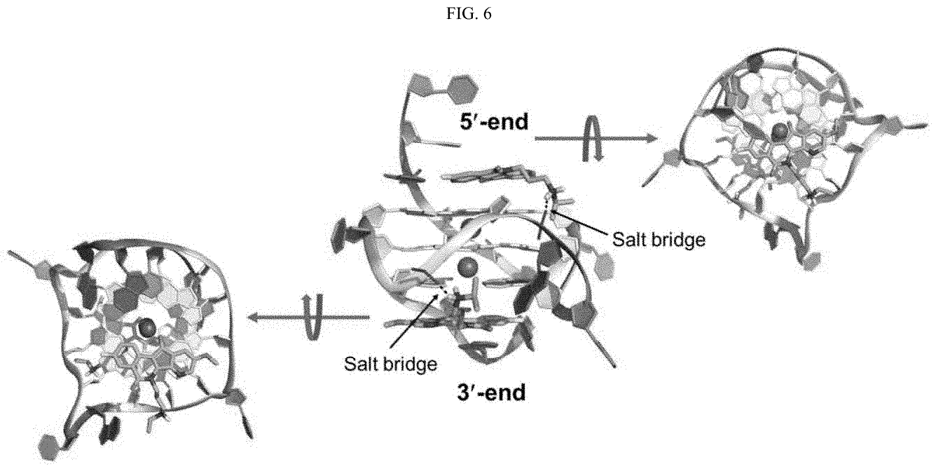

NMR titration data showed that 7-azaindenoisoquinoline 5 (page 24) binds MycG4 to form a well-defined complex at both the 5′- and 3′-ends, as is evident by the significant shifting of the imino proton peaks of the 5′- and 3′-external tetrad guanines (FIG. 5A). We have previously determined the NMR structure of the 2:1 quindoline:MycG4 complex in K⁺ solution (PDB ID 2L7V), in which quindoline binds MycG4 at both ends to form a 5′-complex and 3′-complex.⁴⁸ As indenoisoquinolines are structurally similar to the quindoline compound (FIG. 1C), we performed a molecular docking study to explore the possible binding modes of 7-azaindenoisoquinoline 5 with the MycG4 based on the NMR structure of the 2:1 quindoline:MycG4 complex. The docking program Glide was used in the standard precision (SP) mode: see Methods.⁶⁰⁻⁶¹ 7-Azaindenoisoquinoline 5 was docked to the binding sites at the two ends of the MycG4 using the 2:1 quindoline:MycG4 complex structure (FIG. 6). Several similar binding poses were predicted by the docking experiment for both the 5′- and 3′-sites. Docking studies gave docking scores for the 5′- and 3′-complexes at −6.69 and −6.08 kcal/mol, respectively. FIG. 6 shows a representative model of the 2:1 7-azaindenoisoquinoline 5:MycG4 complex. The overall binding modes of the indenoisoquinoline resembled those of quindoline in the NMR structure of the 2:1 quindoline:MycG4 complex, in which a flanking DNA base from the 5′- or 3′-flanking segment was recruited to form a ligand-base plane stacking over the external tetrads, except that no H-bond was present in the 3′-complex between the indenoisoquinoline and the recruited base. Notably, the tetracyclic ring scaffold of 7-azaindenoisoquinoline 5 with A- and D-ring substituents stacks very well with both the 5′- and 3′-external tetrads, making extensive stacking interactions. The positively charged amine side chain of indenoisoquinoline 5 resides in the MycG4 groove and forms intermolecular salt bridges with phosphate groups on the nucleotide backbone.

### Binding Selectivity of MYC G4-Interactive Indenoisoquinolines and 7-Azaindenoisoquinolines.

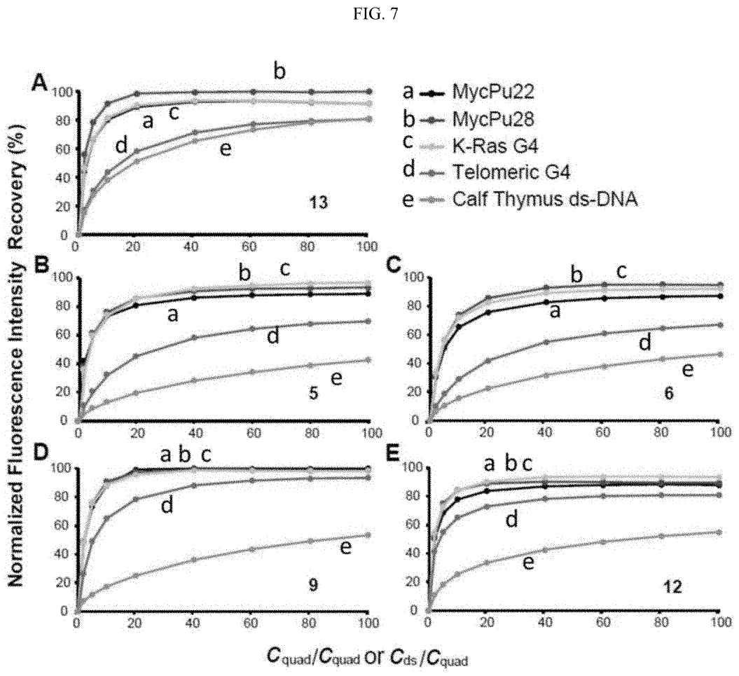

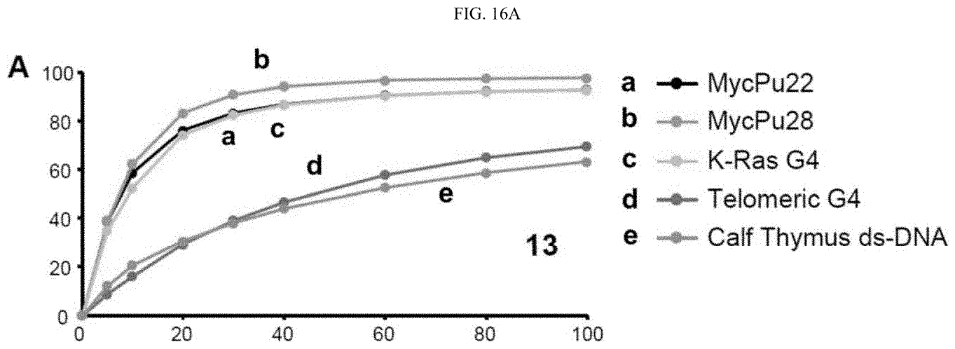

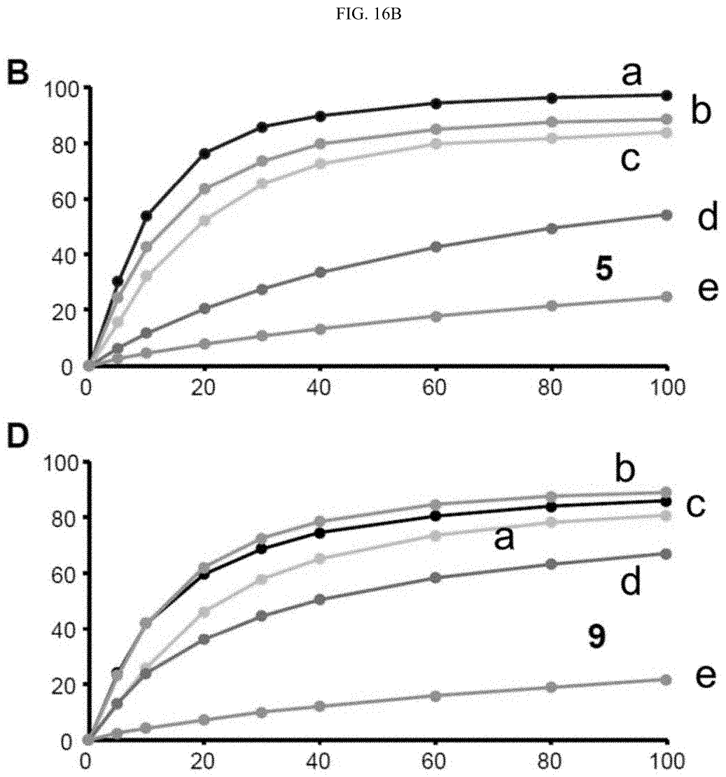

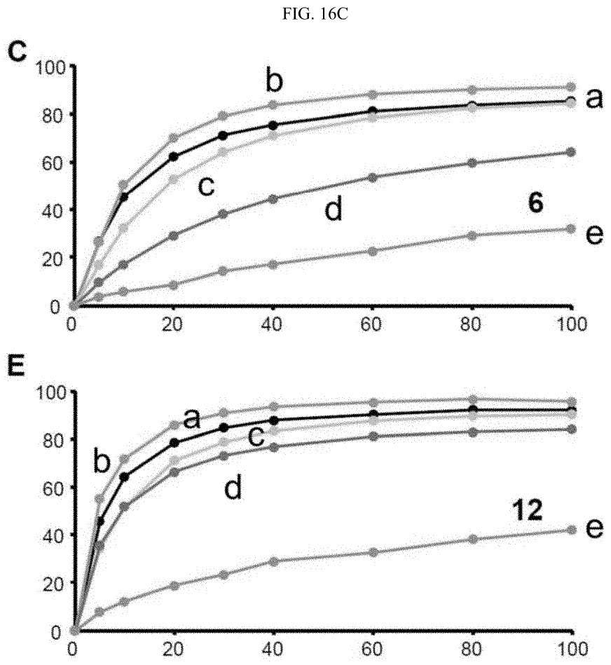

Using a competition fluorescence displacement assay, the binding selectivity of five indenoisoquinolines for MycG4 was determined as compared to a parallel K-Ras promoter G4, a hybrid telomeric G4, and double-stranded (ds) DNA at 1 and 5 equivalents of each compound (FIGS. 7 and 16A-16C). The 3′-TAMRA labeled MycPu22 DNA was used as the fluorescence probe, whose fluorescence was quenched upon the binding of indenoisoquinolines. Upon addition of unlabeled, non-fluorescent competitors (e.g. other DNA G4s and dsDNA), the TAMRA-labeled MycPu22 DNA is displaced by the competitor DNA for indenoisoquinoline binding and the initial high TAMRA-fluorescence is restored. The competition fluorescence displacement assay allows for a straightforward assessment of selective binding towards MycG4 vs. the competitors, i.e. MycG4s (parallel), K-Ras G4 (parallel), telomeric G4 (hybrid), and dsDNA. One and five compound equivalents were used to assess the selectivity of the strongest binding site and other binding sites of each indenoisoquinoline. A quantitative comparison of the competitor affinities (K, values) of five indenoisoquinolines are summarized in Table 2. As shown in FIGS. 7 and 16A-16C, all five MycG4-interactive indenoisoquinolines showed marked binding selectivity for parallel G4s (MycG4s and K-Ras G4) over dsDNA (FIG. 7), and this selectivity became more pronounced at higher compound ratio (FIGS. 16A-16C). Significantly, four 7-azaindenoisoquinolines, 5, 6, 9, and 12, showed remarkable selectivity for DNA G4s over dsDNA (Table 2). However, indenoisoquinoline 13, which has only N6-substitution but no A- and D-ring substituents, showed much less selectivity against dsDNA. This result suggested that substituents on the A- and D-rings are important for selective binding of G4s vs dsDNA. As shown in the modeling study, the substituents on the A- and D-rings of indenoisoquinolines likely contribute to binding MycG4 by more optimal stacking interactions with the external G-tetrads. On the other hand, the increased size of the indenoisoquinoline ring system may hinder intercalation in dsDNA due to possible steric collision with the DNA backbone. Interestingly, the 3-fluoro-substituted 7-azaindenoisoquinolines 5 and 6 showed marked selectivity for parallel G4s over hybrid G4, whereas the 3-nitro-substituted 7-azaindenoisoquinolines 9 and 12 showed much less selectivity, suggesting that the 3-nitro-group may contribute to a less-specific interaction. The less-specific interaction of 7-azaindenoisoquinolines 9 and 12 was also supported by the NMR titration data showing less well-defined MycG4 complexes formed with 9 and 12 (FIG. 5). Albeit with low selectivity against dsDNA, 6-substituted indenoisoquinoline 13 showed selectivity for parallel G4s over hybrid G4. 6-Substituted indenoisoquinolines were previously reported to bind to the c-Kit promoter G4s which were also primarily parallel.¹⁷

### Structure-Activity Relationship of MYC G4 Binding by Indenoisoquinolines.

To understand the factors that govern indenoisoquinoline recognition for MycG4, indenoisoquinoline analogues were analyzed for their MycG4 interactions and MYC inhibitory activity. Trends could be established to generate structure-activity relationships for MycG4 binding (FIG. 4). It was shown that N6-substituents play a critical role in MycG4 binding and stabilization (FIGS. 4A-B). For example, indenoisoquinoline 47 with an N6-dimethylaminopropyl moiety, showed medium MycG4 stabilizing activity, whereas indenoisoquinolines 52 and 53, which lack the aminopropyl side chain structure, were found to be poor MycG4 binders and stabilizers. This suggests that an alkyl amine-containing side chain at N6 of ring B may be important for MycG4 binding (FIG. 4A), possibly due to the favorable electrostatic interactions between the positively charged N-containing side chain and the negatively charged phosphate backbone in the groove of MycG4 at physiological pH 7.4. However, this favorable interaction (compound 13) appears to be weakened by a more bulky N-containing ring-system (compound 16), and abolished by an aromatic N-containing ring-system (compound 17, reduced positive charge for N) (FIG. 4B), suggesting that the bulky nitrogen-containing group may sterically hinder the binding.

9-Methoxy-7-azaindenoisoquinolines, which were developed to improve water solubility and increased charge-transfer properties,⁶²⁻⁶³ appear to bind MycG4 well and show potent MYC-inhibitory activity (FIG. 4C). 7-Azaindenoisoquinolines with small substituents, such as 3-fluoro-, 3-nitro-, and 3-chloro, on the A-ring were found to be strong MycG4 binders and stabilizers and showed potent MYC-inhibitory activity.

### CONCLUSION

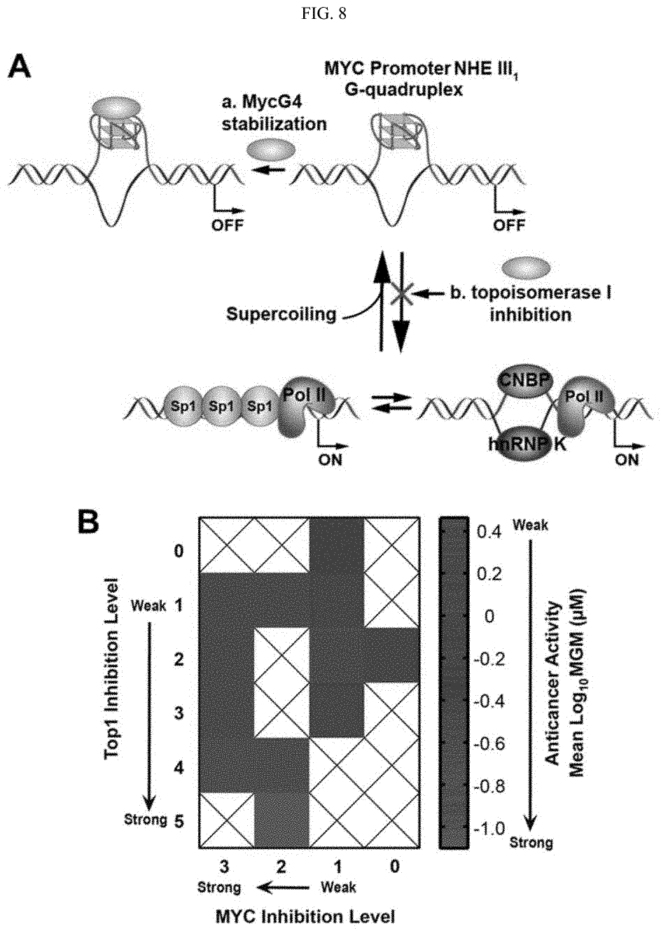

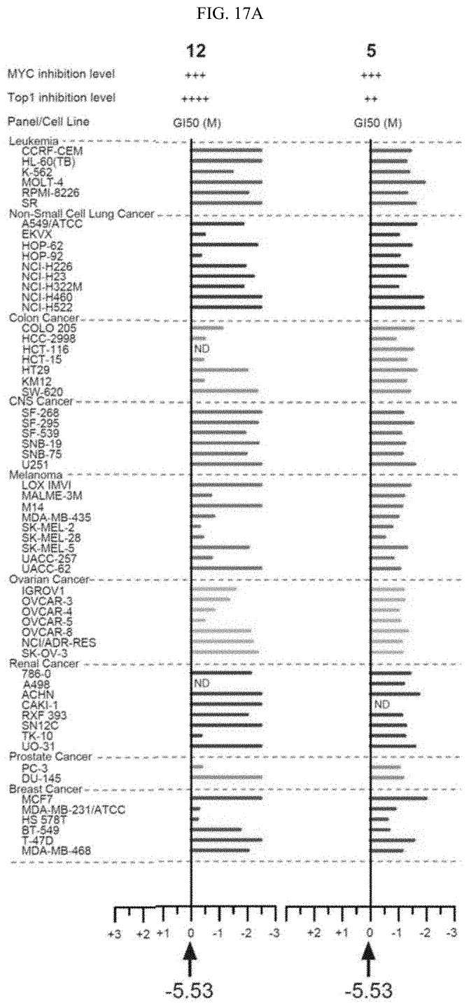

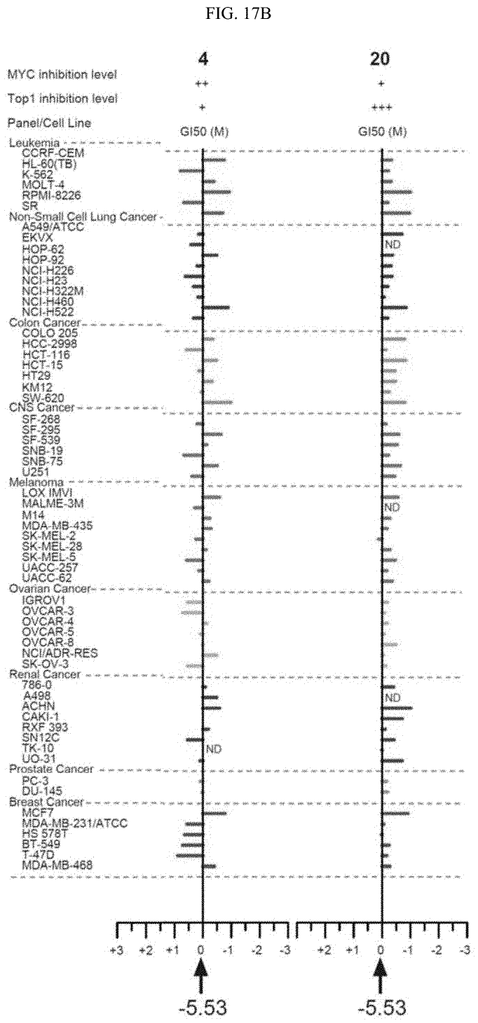

It has been discovered that anticancer indenoisoquinolines and 7-azaindenoisoquinolines strongly bind and stabilize MycG4 and lower MYC levels in cancer cells as revealed by various biophysical, biochemical, computer modeling, and cell-based experiments. A large number of active indenoisoquinolines and 7-azaindenoisoquinolines caused strong MYC downregulation. Indenoisoquinoline analogs are clinically useful anticancer drugs and present a promising scaffold for MycG4-targeting anticancer drug development (FIG. 8A). Insights into structure-activity-relationships of MycG4 recognition by indenoisoquinolines were discovered. Some active indenoisoquinolines and 7-azaindenoisoquinolines were shown to cause both MYC downregulation and topoisomerase I inhibition. Analysis of indenoisoquinoline analogues for their MYC-lowering activity, topoisomerase I inhibitory activity, and anticancer activity led to the discovery of a synergistic effect of MYC-lowering and topoisomerase I inhibition on anticancer activity (FIG. 8, panel B, and FIGS. 17A and 17B). Notably, topoisomerase I specifically relaxes transcription-induced negative supercoiling.⁴ Transcription-induced negative supercoiling is the key to the formation of the MYC promoter G4 (FIG. 8A). Inhibition of the relaxation of transcription-induced negative supercoiling by indenoisoquinolines may promote the formation of MYC promoter G4, which can be further stabilized by the binding of indenoisoquinolines Dual targeting of MycG4 and topoisomerase I was found be an effective mechanism of action for cancer intervention. It is believed that inhibition of the topoisomerase 1 induced relaxation of DNA supercoiling by indenoisoquinolines may be due to the sequestering of the topoisomerase I in the indenoisoquinoline-DNA-topoisomerase ternary complex. Collectively, the results uncover a novel mechanism of action of the clinically useful indenoisoquinoline scaffold as a new family of drugs targeting MycG4 for MYC downregulation. Patients with cancers that are both MYC-positive and MYC promoter G4 positive may benefit from treatment by indenoisoquinolines even more than patients with cancers that are not both MYC-positive and MYC promoter G4 positive. (see following sections). This discovery also suggests that dual targeting of the MYC promoter G4 and topoisomerase I may serve as a novel strategy for anticancer drug development.

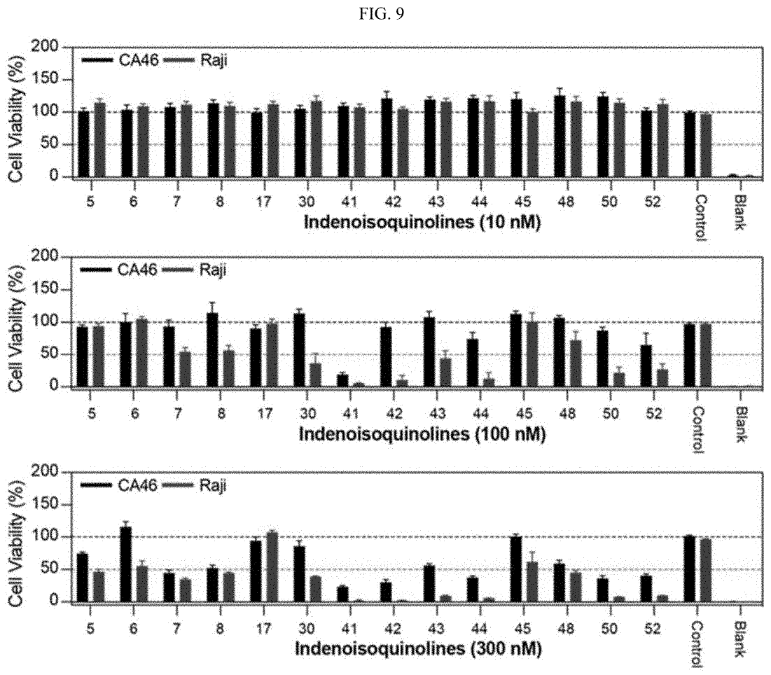

Indenoisoquinolines are More Effective in MYC-Positive Cancers that Contain MYC Promoter G4 (MYC G4) (FIG. 9)

In human Burkitt's lymphoma (BL), a translocation occurs between the IgH heavy chain (an immunoglobin chain) that resides on chromosome 14 and the MYC promoter on chromosome 8, which results in aberrant control and up-regulation of MYC expression^{32, 34, 67}. The BL cell line Raji retains the MYC G4 after the translocation, whereas the BL cell line CA46 losses this element. For characterization of the intracellular activity of indenoisoquinolines, this pair of BL cell lines was used^{32, 34, 67}. It was found that Raji cells are more sensitive than the CA46 cells to indenoisoquinolines that mediate their intracellular effects through stabilization of MYC G4 and modulation of MYC expression. Using MTS assay, active G4-interactive indenoisoquinolines showed dose-dependent cytotoxicity in Raji and CA46 cells, while the Raji cells were much more sensitive than the CA46 cells at 100 nM and 300 nM treatments for 72 h. This data indicates that active G4-interactive indenoisoquinolines target MYC G4 and are more effective against MYC-positive cancers that contain MYC G4. Using these same techniques, differential activity of (aza)indenoisoquinolines against other cancers that are MYC-positive and contain MYC G4 can be measured.

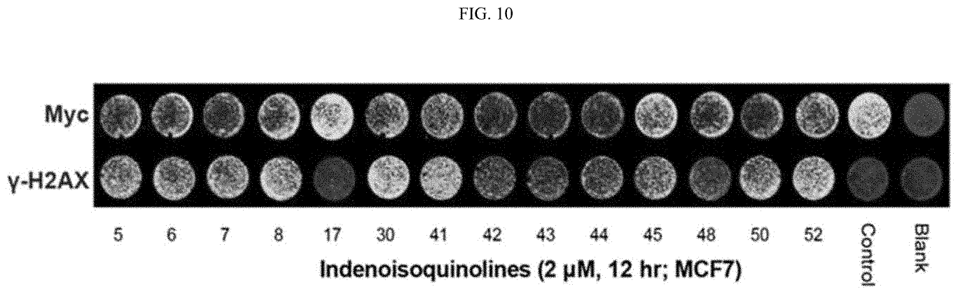

### The More Potent Anticancer Indenoisoquinolines Show Strong MYC Suppression (FIG. 10)

MCF7 is a MYC-positive breast cancer cell line that contains MYC G4. This cell line was used to examine the activities of indenoisoquinolines and dual targeting of MYC and topoisomerase I. Using protein quantification of western blot combined with the high throughput ELISA, in-cell western blot performed in 96-well microplates allow for the assessment of protein expression levels of interest upon drug treatments. We used 96-well in-cell western blot to examine the levels of MYC and phosphorylated form of H2AX (γ-H2AX), a biomarker of DNA double-strand break, upon the treatment with indenoisoquinolines. It was found that potent anticancer G4-interactive indenoisoquinolines significantly decreased the MYC protein expression levels (top panel). In the meantime, active indenoisoquinolines induced the phosphorylated form of H2AX (γ-H2AX) (bottom panel). Therefore, the results show potent anticancer indenoisoquinolines correlate with strong MYC suppression and dual-targeting of topoisomerase I. These results demonstrate strong MYC-suppression of G4-interactive indenoisoquinolines may lead to more potent anticancer activity in MYC-positive cancers that contain MYC G4.

### Materials and Methods

### Sample Preparation.

Unlabeled DNA sequences used for NMR and competition fluorescence displacement assays were synthesized and purified using commercially available reagents as previously described.^{48, 64} The sequences are listed in Table 1. 3′-6-Carboxytetramethylrhodamine (3′-TAMRA)-labeled MycPu22 and 3′-TAMRA, 5′-6-carboxyfluorescein (5′-FAM) dual-labeled MycPu22 DNA sequences were obtained from Sigma-Aldrich. 3′-FAM, 5′-Black Hole Quencher-1 (5′-BHQ1) dual-labeled MycPu28 DNA sequence was synthesized using an Expedite 8909 DNA Synthesizer, with 3′-(6-FAM) CPG (20-2961-xx) and BHQ-1 phosphoramidite (10-5931-xx) obtained from Glen Research Corporation. The synthesized 5′-BHQ1-MycPu28-FAM-3′ DNA sequence was purified using MicroPure II columns and dialyzed against water before lyophilization. DNA concentrations were quantified by UV/Vis absorption at 260 nm using their extinction coefficients. Calf thymus DNA was purchased from Sigma-Aldrich. Indenoisoquinoline stock solutions were dissolved in DMSO at 40 mM by quantifying the mass. For all experiments, indenoisoquinoline stock solutions were further diluted with DMSO or desired buffers.

### Fluorescence Resonance Energy Transfer (FRET) Experiments.

FRET-quenching experiments. The stock solution containing 100 μM 3′-FAM (Ex. 490 nm/Em. 520 nm), 5′-BHQ1 (Abs. 480-580 nm) dual-labeled MycPu28 DNA sequence was first diluted to 2 μM using 50 mM Tris.acetate buffer, pH 7.0. The 2 μM probe solution was equilibrated for 1 h at room temperature. Subsequently, the FRET probe (1 μM) was incubated with the indenoisoquinolines (10 μM) or KCl (100 mM) in 50 mM Tris.acetate buffer at pH 7.0 for another 1 h, using a black 96-well plate (ThermoFisher Scientific) with a total volume of 100 in each well. Fluorescence measurements were then recorded by a Synergy Neo2 plater reader (Bio Tek) at 25° C. with 10 nm bandwidth. The excitation and emission wavelengths were set to 490 and 520 nm, respectively. The final fluorescence intensity was plotted as the average relative fluorescence intensity of two individual experiments after correction for background. Relative fluorescence intensity (%)=F_{Compound}/F_{DMSO}×100%. Relative fluorescence reduction (%)=(1−F_{Compound}/F_{DMSO})×100%.

FRET-melting experiments. The stock solution containing 100 μM 3′-TAMRA (Ex. 555 nm/Em. 580 nm), 5′-FAM (Ex. 490 nm/Em. 520 nm) dual-labeled MycPu22 DNA sequence was first diluted to 2 μM using 7.5 mM KCl, 2.5 mM phosphate buffer, pH 7.0. The 2 μM probe solution was heated to 95° C. for 1 min then cooled down slowly to room temperature for G4 formation. Subsequently, the FRET probe (150 nM) was incubated with the indenoisoquinolines (1.5 μM) in 7.5 mM KCl, 2.5 mM phosphate buffer at pH 7.0 for 1 h, using a blank 96-well plate (ThermoFisher Scientific) with a total volume of 100 μL for each well. In the presence of 10 mM K⁺, the labeled MycPu22 is mainly present in a G4 form where the FAM is in close proximity to the TAMRA, which shows a low FAM fluorescence due to the FRET effect. With gradually increasing temperature, the MycPu22 DNA is unfolded from the G4 form to a single-stranded conformation where the FAM is far apart to the TAMRA, which results in a high FAM fluorescence. Melting curves for the determination of Tₘ were then obtained by recording FAM fluorescence with increasing temperatures from 25 to 95° C. at a rate of 0.9° C./min using a QuantStudio 6 Flex Real-Time PCR System. The Tₘ values were determined by the maximum of the first derivative plot of the melting curves. The final Tₘ values were plotted as the average Tₘ values of two individual experiments.

### Cell Culture.

MCF-7 (Michigan Cancer Foundation-7) cancer cell lines were originally obtained from the Arizona Cancer Center and grown in RMPI 1640 (10-040-CV, Corning) supplemented with 10% fetal bovine serum (35-010-CV, Corning). Cells were incubated at 37° C. with 5% CO₂.

### Western Blotting.

After collecting cells from 6-well plates, the cell pellets were re-suspended in 150 μL of 1×RIPA buffer supplemented with 1× Protease Inhibitor Cocktail (11836153001, Roche) and 1× NuPAGE LDS Sample Buffer (NP0007, Invitrogen) and then proteins were immediately denatured at 80° C. for 10 min. After sonication, 7 μL of each sample was analyzed using 4-15% Mini-PROTEAN TGX Gels (456-1086, Bio-Rad). The gels were cut into strips that contained the proteins of interest and transferred to nitrocellulose membrane (I323002, Invitrogen) using an iBlot 2 Dry Transfer Device (Invitrogen). Immunoblotting was carried out according to standard procedures using the ECL detection method. The membrane was hybridized with the following antibodies: monoclonal anti-MYC (1:1000 dilution; rabbit, Cell Signaling Technology), monoclonal anti-GAPDH (1:2000 dilution; rabbit, Cell Signaling Technology).

### NCI-60 Cancer Cell Line Drug Screen.

The antiproliferative activities of the indenoisoquinoline compounds were determined in the NCI-60 cancer cell lines of the National Cancer Institute Developmental Therapeutics Program (NCI-DTP) (Table 3).⁵³⁻⁵⁵ Compounds showed sufficient cytotoxicity during the pre-screen were subjected to the five-dose assay to determine the 50% growth inhibition (GI₅₀) values. Cancer cells were incubated with the test compounds at five concentrations ranging from 100 μM to 10 nM for 48 h. After the treated cancer cells had been stained with sulforhodamine B dye, the percentage growth was plotted as a function of the common logarithm of the tested compound concentration. The GI₅₀ values were determined by interpolation between the points located above and below the 50% cell growth. GI₅₀ values above and below the tested range (10⁻⁴ to 10⁻⁸ M) were taken as the maximum (10⁻⁴ M) and minimum (10⁻⁸ M) drug concentrations, respectively, used in the screening test. The approximate average of GI₅₀ values across the entire panel of NCI-60 cancer cell lines for each compound was recorded as the MGM value.

### Quantitative Reverse Transcription PCR (QRT-PCR).

Total RNA was isolated using TRIzol reagent (Invitrogen). To remove phenol contamination, purified RNA was dissolved in DEPC-treated water and re-precipitated with 75% ethanol. RNA (1 μg) was subjected to cDNA synthesis using the qScript cDNA Synthesis kit (Quanta Biosciences) according to manufacturer's instructions. Real-time PCR was performed in triplicate reactions. For each reaction, a mix of the following reaction components was prepared to the indicated end-concentration: 3 μl water, 1 μl cDNA synthesis products, 0.25 μM of each primer for MYC or GAPDH and 5 μl of SYBR Green PCR Master Mix. Cycling conditions were 95° C. for 5 min, followed by 40 cycles of 95° C. for 15 s, 60° C. for 15 s and 72° C. for 15 s. Relative gene expression was calculated by using the 2^{-ΔΔCT}, in which the amount of MYC mRNA was normalized to an endogenous reference (GAPDH). Melting curve analysis or agarose gel electrophoresis was carried out to confirm correct PCR products.

### Nuclear Magnetic Resonance (NMR) Spectroscopy Experiments.

All NMR experiments were conducted using a Bruker AV-500 spectrometer equipped with a Prodigy cryoprobe at 25° C. Watergate water suppression technique was used to suppress water signals. Briefly, each DNA sample was prepared to a final concentration of 150 oligonucleotide in 75 mM KCl, 25 mM phosphate buffer at pH 7.0, and containing 90/10% H₂O/D₂O. DNA samples were heated to 95° C. for 5 min then cooled slowly to room temperature for G4 formation. ¹H-NMR titrations were performed by adding increasing amounts of the compound (0.5 to 4 equivalents) to the oligonucleotide solution.

### Native Gel Electrophoretic Mobility Shift Assay (EMSA).

Native PAGE experiments were performed with a 1.5 mm thick 10×7 cm native gel containing 15% acrylamide (acrylamide:bisacrylamide 29:1) in 1×TBE buffer, pH 8.0, supplemented with 12.5 mM KCl. MycG4 DNA samples were the samples from NMR titration experiments in the absence and presence of indenoisoquinolines. Each sample contains 4 μL of 150 μM DNA. DNA bands were visualized using ultraviolet (UV) light absorption at 260 nm.

### Circular Dichroism (CD) Spectroscopy Experiments.

Circular dichroism spectra were recorded using a Jasco-1100 spectropolarimeter (Jasco Inc.) equipped with a temperature controller. Samples were prepared in 3.8 mM KCl, 1.2 mM phosphate buffer at a DNA concentration of 15 μM in the absence and presence of the indenoisoquinolines. CD measurements were taken through a quartz cell with a 1 mm path length, 1 nm bandwidth, and 1 s response time for spectra at 25° C. Spectra were obtained using three averaged scans between 230 and 330 nm. The baseline was corrected by subtracting the buffer spectrum.

### Fluorescence-Based Binding Assay.

The fluorescence-based binding assay was performed on a Jasco FP-8300 spectrofluorometer equipped with a temperature controller at 20° C. The stock solution containing 2 μM 3′-TAMRA labelled MycPu22 oligonucleotide was diluted to 0.5 nM using 75 mM KCl, 25 mM phosphate buffer, pH 7.0. To check the binding affinity of each indenoisoquinoline to MycG4 DNA, the compound was gradually added to the DNA solution in a volume of 1.6 mL using a quartz cell with a 10 mm path length. After each addition of the compound, the solution was allowed to equilibrate for at least 2 min. The fluorescence spectrum was recorded at a range from 570 to 600 nm with an excitation wavelength of 555 nm, 10 nm bandwidths, 100 nm/min scan speed, and 1 s response time. The fluorescence intensity at the emission maximum (λₘₐₓ=580 nm) was used in all calculations. The apparent binding affinity K_{d} values were determined by fitting the data to a one site-specific binding model using GraphPad Prism software, with a simplified equation of

$$Δ {F}_{o b s} = Δ {F}_{max} \frac{{[ L ]}_{T}}{{[ L ]}_{T} + {K}_{d , app}} , \qquad$$

where ΔF represents the fluorescence intensity change of the indenoisoquinolines bound to MycPu22 DNA and [L]_{T} represents the total ligand concentration that is the independent variable, varying with each measurement.

### Competition Fluorescence Displacement Experiments.

The competition fluorescence displacement experiments were performed on a Jasco FP-8300 Spectrofluorometer equipped with a temperature controller at 20° C. The stock solution containing 2 μM 3′-TAMRA-labelled MycPu22 oligonucleotide was diluted to 20 nM using 75 mM KCl, 25 mM phosphate buffer at pH 7.0. To check the binding selectivity of each compound to MycG4 DNA, 20 nM or 100 nM of the indenoisoquinoline was added to the DNA solution in a total volume of 1.6 mL in a quartz cell with a 10 mm path length. Subsequently, various unlabeled MycG4s (MycPu22 and MycPu28), K-Ras G4, telomeric G4 DNAs, or calf thymus dsDNA were gradually added to the complex solution. For each addition of the DNA, the sample was equilibrated at least 2 min. The fluorescence spectrum was recorded between 570 and 600 nm with an excitation wavelength of 555 nm, 10 nm bandwidths, 100 nm/min of scan speed, and 1 s of response time. The fluorescence intensities at the emission maximum (Δₘₐₓ=580 nm) were plotted for figures. The competitor binding affinities (K, values) were calculated by

$${K}_{i} = \frac{{C}_{50}}{1 + \frac{{ }_{[ L ]}}{{K}_{d , a p p}}} , \qquad$$

using data from 20 nM compound. The C₅₀ value was the concentration of the unlabeled competing DNA that recovers the fluorescence of the labelled DNA by 50%. [L] represents the ligand concentration that is a constant value of 20 nM. K_{d,app} values were obtained by fluorescence-based binding assay.

### Molecular Modeling

The binding sites at the two ends of the MycG4 were defined by using the NMR structure of the 2:1 quindoline:MycG4 complex (PDB ID 2L7V).⁴⁸ The docking program Glide (Schrödinger Inc.) was used in the standard precision (SP).⁶⁰⁻⁶¹ During docking, the DNA was fixed while the ligand was flexible. Before running docking, the 3D energy-minimized structure of the ligand was generated using the LigPrep from Maestro (Schrödinger Inc.). The protonation state of the ligand was assigned at pH 7.0 using the program Epik (Schrödinger Inc.).⁶⁵ The following default settings in the Glide protocol were used for docking: the OPLS3 force field was used to describe the DNA-ligand complex and a distance-dependent dielectric constant ε=2.0 was used to mimic the solvent effect.⁶⁶ A maximum of 5000 poses passed through the initial phase of docking, and a maximum of 400 best poses were kept for energy minimization. The maximum number of the minimization steps was set to be 100.

Those skilled in the art will recognize that numerous modifications can be made to the specific implementations described above. The implementations should not be limited to the particular limitations described. Other implementations may be possible.

While the inventions have been illustrated and described in detail in the drawings and foregoing description, the same is to be considered as illustrative and not restrictive in character, it being understood that only certain embodiments have been shown and described and that all changes and modifications that come within the spirit of the invention are desired to be protected.

It is intended that that the scope of the present methods and compositions be defined by the following claims. However, it must be understood that this disclosure may be practiced otherwise than is specifically explained and illustrated without departing from its spirit or scope. It should be understood by those skilled in the art that various alternatives to the embodiments described herein may be employed in practicing the claims without departing from the spirit and scope as defined in the following claims.

### REFERENCES

1. Kohlhagen, G.; Paull, K. D.; Cushman, M.; Nagafuji, P.; Pommier, Y., Protein-linked DNA strand breaks induced by NSC 314622, a novel noncamptothecin topoisomerase I poison. Mol. Pharmacol. 1998, 54 (1), 50-58.
2. Strumberg, D.; Pommier, Y.; Paull, K.; Jayaraman, M.; Nagafuji, P.; Cushman, M., Synthesis of cytotoxic indenoisoquinoline topoisomerase I poisons. Synthesis of cytotoxic indenoisoquinoline topoisomerase I poisons. J. Med. Chem. 1999, 42 (3), 446-457.
3. Cushman, M.; Jayaraman, M.; Vroman, J. A.; Fukunaga, A. K.; Fox, B. M.; Kohlhagen, G.; Strumberg, D.; Pommier, Y., Synthesis of new indeno [1, 2-c] isoquinolines: cytotoxic non-camptothecin topoisomerase I inhibitors. J. Med. Chem. 2000, 43 (20), 3688-3698.
4. Pommier, Y.; Sun, Y.; Shar-yin, N. H.; Nitiss, J. L., Roles of eukaryotic topoisomerases in transcription, replication and genomic stability. Nat. Rev. Mol. Cell. Biol. 2016, 17 (11), 703-721.
5. Pommier, Y., DNA topoisomerase I inhibitors: chemistry, biology, and interfacial inhibition. Chem. Rev. 2009, 109 (7), 2894-2902.
6. Wang, P.; Elsayed, M. S. A.; Plescia, C. B.; Ravji, A.; Redon, C. E.; Kiselev, E.; Marchand, C.; Zeleznik, O.; Agama, K.; Pommier, Y.; Cushman, M., Synthesis and biological evaluation of the first triple inhibitors of human topoisomerase I, tyrosyl-DNA phosphodiesterase 1 (Tdp1), and tyrosyl-DNA phosphodiesterase 2 (Tdp2). J. Med. Chem. 2017, 60 (8), 3275-3288.
7. Elsayed, M. S. A.; Su, Y.; Wang, P.; Sethi, T.; Agama, K.; Ravji, A.; Redon, C. E.; Kiselev, E.; Horzmann, K. A.; Freeman, J. L.; Pommier, Y.; Cushman, M., Design and synthesis of chlorinated and fluorinated 7-azaindenoisoquinolines as potent cytotoxic anticancer agents that inhibit topoisomerase I. J. Med. Chem. 2017, 60 (13), 5364-5376.
8. Cinelli, M. A.; Reddy, P. V.; Lv, P. C.; Liang, J. H.; Chen, L.; Agama, K.; Pommier, Y.; van Breemen, R. B.; Cushman, M., Identification, synthesis, and biological evaluation of metabolites of the experimental cancer treatment drugs indotecan (LMP400) and indimitecan (LMP776) and investigation of isomerically hydroxylated indenoisoquinoline analogues as topoisomerase I poisons. J. Med Chem. 2012, 55 (24), 10844-10862.
9. Nagarajan, M.; Morrell, A.; Ioanoviciu, A.; Antony, S.; Kohlhagen, G.; Agama, K.; Hollingshead, M.; Pommier, Y.; Cushman, M., Synthesis and evaluation of indenoisoquinoline topoisomerase I inhibitors substituted with nitrogen heterocycles. J. Med Chem. 2006, 49 (21), 6283-6289.
10. Doroshow, J. H.; Ji, J. J.; Chen, A.; Allen, D.; Zhang, Y.; Lawrence, S. M.; Pfister, T. D.; Wang, L.; Redon, C. E.; Bonner, W.; Speranza, G.; Weil, M. K.; Eiseman, J.; Holleran, J. L.; Kinders, R. J.; Beumer, J. H.; Parchment, R. E.; Pommier, Y.; Tomaszewski, J. E.; Kummar, S., Proof of mechanism (POM) in the first-in-human trial of two novel indenoisoquinoline, non-camptothecin topoisomerase I (TOP1) inhibitors. J. Clin. Oncol. 2012, 30 (15 suppl), 3031-3031.
11. Kummar, S.; Chen, A.; Gutierrez, M.; Pfister, T. D.; Wang, L.; Redon, C.; Bonner, W. M.; Yutzy, W.; Zhang, Y.; Kinders, R. J., Clinical and pharmacologic evaluation of two dosing schedules of indotecan (LMP400), a novel indenoisoquinoline, in patients with advanced solid tumors. Cancer Chemother. Pharmacol. 2016, 78 (1), 73-81.
12. Coyne, G. H. O. S.; Kummar, S.; Meehan, R. S.; Streicher, H.; Takebe, N.; Sharon, E.; Conley, B. A.; Harris, L.; Collins, J. M.; Moore, N.; Juwara, L.; Rubinstein, L.; Quinn, M. F.; Pommier, Y.; Cushman, M.; Doroshow, J. H.; Chen, A. P., Phase I study of indenoisoquinolines LMP776 in adults with relapsed solid tumors and lymphomas. J. Clin. Oncol. 2017, 35 (15 suppl), 2558-2558.
13. A phase I study of indenoisoquinolines LMP400 and LMP776 in adults with relapsed solid tumors and lymphomas. https://clinicaltrials.govict2/show/NCT01051635 (Accessed Apr. 20, 2019).
14. Indenoisoquinoline LMP744 in adults with relapsed solid tumors and lymphomas. https://clinicaltrials.govict2/show/NCT03030417 (accessed Apr. 20, 2019).
15. Nagarajan, M.; Morrell, A.; Fort, B. C.; Meckley, M. R.; Antony, S.; Kohlhagen, G.; Pommier, Y.; Cushman, M., Synthesis and anticancer activity of simplified indenoisoquinoline topoisomerase I inhibitors lacking substituents on the aromatic rings. J. Med. Chem. 2004, 47 (23), 5651-5661.
16. Pommier, Y.; Cushman, M., The indenoisoquinoline noncamptothecin topoisomerase I inhibitors: update and perspectives. Mol. Cancer. Ther. 2009, 8 (5), 1008-1014.
17. Bejugam, M.; Gunaratnam, M.; Müller, S.; Sanders, D. A.; Sewitz, S.; Fletcher, J. A.; Neidle, S.; Balasubramanian, S., Targeting the c-Kit promoter G-quadruplexes with 6-substituted indenoisoquinolines. ACS Med. Chem. Lett. 2010, 1 (7), 306-310.
18. Slack, G. W.; Gascoyne, R. D., MYC and aggressive B-cell lymphomas. Adv. Anat. Pathol. 2011, 18 (3), 219-228.
19. Nesbit, C. E.; Tersak, J. M.; Prochownik, E. V., MYC oncogenes and human neoplastic disease. Oncogene 1999, 18 (19), 3004-3016.
20. Dang, C. V., MYC on the path to cancer. Cell 2012, 149 (1), 22-35.
21. Bouvard, C.; Lim, S. M.; Ludka, J.; Yazdani, N.; Woods, A. K.; Chatterjee, A. K.; Schultz,
P. G.; Zhu, S., Small molecule selectively suppresses MYC transcription in cancer cells. Proc. Natl. Acad. Sci. U.S.A 2017, 114 (13), 3497-3502.
22. Adhikary, S.; Eilers, M., Transcriptional regulation and transformation by MYC proteins. Nat. Rev. Mol. Cell. Biol. 2005, 6 (8), 635-645.
23. Meyer, N.; Penn, L. Z., Reflecting on 25 years with MYC. Nat. Rev. Cancer 2008, 8 (12), 976-990.
24. Dang, C. V., c-MYC target genes involved in cell growth, apoptosis, and metabolism. Mol. Cell. Biol. 1999, 19 (1), 1-11.
25. Lin, C. Y.; Lovén, J.; Rahl, P. B.; Paranal, R. M.; Burge, C. B.; Bradner, J. E.; Lee, T. I.; Young, R. A., Transcriptional amplification in tumor cells with elevated c-MYC. Cell 2012, 151 (1), 56-67.
26. Nie, Z.; Hu, G.; Wei, G.; Cui, K.; Yamane, A.; Resch, W.; Wang, R.; Green, D. R.; Tessarollo, L.; Casellas, R.; Zhao, K.; Levens, D., c-MYC is a universal amplifier of expressed genes in lymphocytes and embryonic stem cells. Cell 2012, 151 (1), 68-79.
27. Jain, M.; Arvanitis, C.; Chu, K.; Dewey, W.; Leonhardt, E.; Trinh, M.; Sundberg, C. D.; Bishop, J. M.; Felsher, D. W., Sustained loss of a neoplastic phenotype by brief inactivation of MYC. Science 2002, 297 (5578), 102-104.
28. Weinstein, I. B.; Joe, A. K., Mechanisms of disease: Oncogene addiction—a rationale for molecular targeting in cancer therapy. Nat. Clin. Pract. Oncol. 2006, 3 (8), 448-457.
29. Dang, C. V.; Reddy, E. P.; Shokat, K. M.; Soucek, L., Drugging the ‘undruggable’ cancer targets. Nat. Rev. Cancer 2017, 17 (8), 502-508.
30. Felsenstein, K. M.; Saunders, L. B.; Simmons, J. K.; Leon, E.; Calabrese, D. R.; Zhang,
S.; Michalowski, A.; Gareiss, P.; Mock, B. A.; Schneekloth, J. S., Jr., Small molecule microarrays enable the identification of a selective, quadruplex-binding inhibitor of MYC expression. ACS Chem. Biol. 2016, 11 (1), 139-148.
31. Whitfield, J. R.; Beaulieu, M. E.; Soucek, L., Strategies to inhibit MYC and their clinical applicability. Front. Cell Dev. Biol. 2017, 5, 1-13.
32. Siddiqui-Jain, A.; Grand, C. L.; Bearss, D. J.; Hurley, L. H., Direct evidence for a G-quadruplex in a promoter region and its targeting with a small molecule to repress c-MYC transcription. Proc. Natl. Acad. Sci. U.S.A 2002, 99 (18), 11593-11598.
33. Brooks, T. A.; Hurley, L. H., The role of supercoiling in transcriptional control of MYC and its importance in molecular therapeutics. Nat. Rev. Cancer 2009, 9 (12), 849-861.
34. Brown, R. V.; Danford, F. L.; Gokhale, V.; Hurley, L. H.; Brooks, T. A., Demonstration that drug-targeted downregulation of MYC in non-hodgkins lymphoma is directly mediated through the promoter G-quadruplex. J. Biol. Chem. 2011, 286 (47), 41018-41027.
35. Kouzine, F.; Sanford, S.; Elisha-Feil, Z.; Levens, D., The functional response of upstream DNA to dynamic supercoiling in vivo. Nat. Struct. Mol. Biol. 2008, 15 (2), 146-154.
36. Kang, H. J.; Park, H. J., Novel molecular mechanism for Actinomycin D activity as an oncogenic promoter G-quadruplex binder. Biochemistry 2009, 48 (31), 7392-7398.
37. Yang, D.; Okamoto, K., Structural insights into G-quadruplexes: towards new anticancer drugs. Future Med Chem. 2010, 2 (4), 619-646.
38. Qin, Y.; Hurley, L. H., Structures, folding patterns, and functions of intramolecular DNA G-quadruplexes found in eukaryotic promoter regions. Biochimie 2008, 90 (8), 1149-1171.
39. Chen, Y.; Yang, D., Sequence, stability, and structure of G-quadruplexes and their interactions with drugs. Curr. Protoc. Nucleic Acid Chem. 2012, Chapter 17, Unit17 5.
40. Balasubramanian, S.; Hurley, L. H.; Neidle, S., Targeting G-quadruplexes in gene promoters: a novel anticancer strategy? Nat. Rev. Drug Discov. 2011, 10 (4), 261-275.
41. Biffi, G.; Tannahill, D.; McCafferty, J.; Balasubramanian, S., Quantitative visualization of DNA G-quadruplex structures in human cells. Nat. Chem. 2013, 5 (3), 182-186.
42. Hansel-Hertsch, R.; Beraldi, D.; Lensing, S. V.; Marsico, G.; Zyner, K.; Parry, A.; Di Antonio, M.; Pike, J.; Kimura, H.; Narita, M.; Tannahill, D.; Balasubramanian, S., G-quadruplex structures mark human regulatory chromatin. Nat. Genet. 2016, 48 (10), 1267-1275.
43. Ambrus, A.; Chen, D.; Dai, J.; Jones, R. A.; Yang, D., Solution structure of the biologically relevant G-quadruplex element in the human c-MYC promoter. Biochemistry 2005, 44 (6), 2048-2058.
44. Mathad, R. I.; Hatzakis, E.; Dai, J.; Yang, D., c-MYC promoter G-quadruplex formed at the 5′-end of NHE III1 element: insights into biological relevance and parallel-stranded G-quadruplex stability. Nucleic Acids Res. 2011, 39 (20), 9023-9033.
45. Seenisamy, J.; Rezler, E. M.; Powell, T. J.; Tye, D.; Gokhale, V.; Joshi, C. S.; Siddiqui-Jain, A.; Hurley, L. H., The dynamic character of the G-quadruplex element in the c-MYC promoter and modification by TMPyP4. J. Am. Chem. Soc. 2004, 126 (28), 8702-8709.
46. Ou, T. M.; Lu, Y. J.; Zhang, C.; Huang, Z. S.; Wang, X. D.; Tan, J. H.; Chen, Y.; Ma, D. L.; Wong, K. Y.; Tang, J. C.; Chan, A. S.; Gu, L. Q., Stabilization of G-quadruplex DNA and down-regulation of oncogene c-MYC by quindoline derivatives. J. Med. Chem. 2007, 50 (7), 1465-1474.
47. Ou, T. M.; Lin, J.; Lu, Y. J.; Hou, J. Q.; Tan, J. H.; Chen, S. H.; Li, Z.; Li, Y. P.; Li, D.; Gu, L. Q., Inhibition of cell proliferation by quindoline derivative (SYUIQ-05) through its preferential interaction with c-MYC promoter G-quadruplex. J. Med. Chem. 2011, 54 (16), 5671-5679.
48. Dai, J.; Carver, M.; Hurley, L. H.; Yang, D., Solution structure of a 2:1 quindoline-c-MYC G-quadruplex: insights into G-quadruplex-interactive small molecule drug design. J. Am. Chem. Soc. 2011, 133 (44), 17673-17680.
49. Hatzakis, E.; Okamoto, K.; Yang, D., Thermodynamic stability and folding kinetics of the major G-quadruplex and its loop isomers formed in the nuclease hypersensitive element in the human c-MYC promoter: effect of loops and flanking segments on the stability of parallel-stranded intramolecular G-quadruplexes. Biochemistry 2010, 49 (43), 9152-9160.
50. Morrell, A.; Placzek, M.; Parmley, S.; Antony, S.; Dexheimer, T. S.; Pommier, Y.; Cushman, M., Nitrated indenoisoquinolines as topoisomerase I inhibitors: a systematic study and optimization. J. Med. Chem. 2007, 50 (18), 4419-4430.
51. Conda-Sheridan, M.; Reddy, P. N.; Morrell, A.; Cobb, B. T.; Marchand, C.; Agama, K.; Chergui, A.; Renaud, A.; Stephen, A. G.; Bindu, L. K., Synthesis and biological evaluation of indenoisoquinolines that inhibit both tyrosyl-DNA phosphodiesterase I (Tdp1) and topoisomerase I (Top1). 1 Med. Chem. 2012, 56 (1), 182-200.
52. Beck, D. E.; Agama, K.; Marchand, C.; Chergui, A.; Pommier, Y.; Cushman, M., Synthesis and biological evaluation of new carbohydrate-substituted indenoisoquinoline topoisomerase I inhibitors and improved syntheses of the experimental anticancer agents indotecan (LMP400) and indimitecan (LMP776). J. Med. Chem. 2014, 57 (4), 1495-1512.
53. NCI-60 human tumor cell lines screen. https://dtp.cancer.gov/discovery_development/nci-60/(accessed Apr. 20, 2019).
54. Kiselev, E.; Sooryakumar, D.; Agama, K.; Cushman, M.; Pommier, Y., Optimization of the lactam side chain of 7-azaindenoisoquinoline topoisomerase I inhibitors and mechanism of action studies in cancer cells. J. Med Chem. 2014, 57 (4), 1289-1298.
55. Shoemaker, R. H., The NCI60 human tumour cell line anticancer drug screen. Nat. Rev. Cancer 2006, 6 (10), 813.
56. Kotar, A.; Wang, B.; Shivalingam, A.; Gonzalez-Garcia, J.; Vilar, R.; Plavec, J., NMR structure of a triangulenium-based long-lived fluorescence probe bound to a G-quadruplex. Angew. Chem., Int. Ed. 2016, 55 (40), 12508-12511.
57. Del Villar-Guerra, R.; Trent, J. O.; Chaires, J. B., G-quadruplex secondary structure obtained from circular dichroism spectroscopy. Angew. Chem., Int. Ed. 2018, 130 (24), 7289-7293.
58. Brown, R. V.; Wang, T.; Chappeta, V. R.; Wu, G.; Onel, B.; Chawla, R.; Quijada, H.; Camp, S. M.; Chiang, E. T.; Lassiter, Q. R., The consequences of overlapping G-Quadruplexes and i-Motifs in the platelet-derived growth factor receptor β core promoter nuclease hypersensitive element can explain the unexpected effects of mutations and provide opportunities for selective targeting of both structures by small molecules to downregulate gene expression. J. Am. Chem. Soc. 2017, 139 (22), 7456-7475.
59. Le, D. D.; Di Antonio, M.; Chan, L.; Balasubramanian, S., G-quadruplex ligands exhibit differential G-tetrad selectivity. Chem. Commun. 2015, 51 (38), 8048-8050.
60. Friesner, R. A.; Banks, J. L.; Murphy, R. B.; Halgren, T. A.; Klicic, J. J.; Mainz, D. T.; Repasky, M. P.; Knoll, E. H.; Shelley, M.; Perry, J. K., Glide: a new approach for rapid, accurate docking and scoring. 1. Method and assessment of docking accuracy. J. Med. Chem. 2004, 47 (7), 1739-1749.
61. Halgren, T. A.; Murphy, R. B.; Friesner, R. A.; Beard, H. S.; Frye, L. L.; Pollard, W. T.; Banks, J. L., Glide: a new approach for rapid, accurate docking and scoring. 2. Enrichment factors in database screening. J. Med. Chem. 2004, 47 (7), 1750-1759.
62. Kiselev, E.; DeGuire, S.; Morrell, A.; Agama, K.; Dexheimer, T. S.; Pommier, Y.; Cushman, M., 7-azaindenoisoquinolines as topoisomerase I inhibitors and potential anticancer agents. J. Med. Chem. 2011, 54 (17), 6106-6116.
63. Kiselev, E.; Agama, K.; Pommier, Y.; Cushman, M., Azaindenoisoquinolines as Topoisomerase I Inhibitors and Potential Anticancer Agents: A Systematic Study of Structure—Activity Relationships. J. Med. Chem. 2012, 55 (4), 1682-1697.
64. Onel, B.; Carver, M.; Wu, G.; Timonina, D.; Kalarn, S.; Larriva, M.; Yang, D., A new G-quadruplex with hairpin loop immediately upstream of the human BCL2 P1 promoter modulates transcription. J. Am. Chem. Soc. 2016, 138 (8), 2563-2570.
65. Shelley, J. C.; Cholleti, A.; Frye, L. L.; Greenwood, J. R.; Timlin, M. R.; Uchimaya, M., Epik: a software program for pK a prediction and protonation state generation for drug-like molecules. J. Computer Aid. Mol. Des. 2007, 21 (12), 681-691.
66. Harder, E.; Damm, W.; Maple, J.; Wu, C.; Reboul, M.; Xiang, J. Y.; Wang, L.; Lupyan, D.; Dahlgren, M. K.; Knight, J. L., OPLS3: a force field providing broad coverage of drug-like small molecules and proteins. J. Chem. Theory Comput. 2015, 12 (1), 281-296.
67. Calabrese, D. R.; Chen, X.; Leon, E. C.; Gaikwad, S. M.; Phyo, Z.; Hewitt, W. M.; Alden, S.; Hilimire, T. A.; He, F.; Michalowski, A. M.; Simmons, J. K.; Saunders, L. B.; Zhang, S.; Connors, D.; Walters, K. J.; Mock, B. A.; Schneekloth, J. S., Jr., Chemical and structural studies provide a mechanistic basis for recognition. Nat Commun 2018, 9 (1), 4229.

We claim:

1. A method for selecting a patient for treatment with a compound of formula (I), or a pharmaceutically acceptable salt, hydrate, or solvate thereof, wherein the patient has a MYC-positive cancer comprising the steps of
a) obtaining a sample of the patient's cancer;
b) determining if the cancer cells in the sample are MYC-positive; and
c) selecting the patient for treatment with the compound of formula (I), or a pharmaceutically acceptable salt, hydrate, or solvate thereof, if the patient's cancer cells in the sample are MYC-positive; 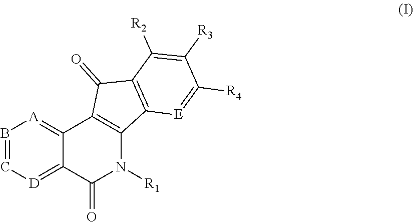
wherein
A is N, CH, or CR; B is N, CH, or CR; C is N, CH, or CR; D is N, CH, or CR; E is N, CH, or CR, wherein R is a halo, azido, alkoxy, cyano, nitro, hydroxy, amino, thio, or a derivative thereof; or an alkyl, alkenyl, heteroalkyl, heteroalkenyl, heterocyclyl, cycloalkyl, cycloalkenyl, cycloheteroalkyl, cycloheteroalkenyl, aryl, arylalkyl, and arylalkenyl, each of which is optionally substituted;
R₁ is an alkyl, alkenyl, heteroalkyl, heteroalkenyl, heterocyclyl, hydroxyalkyl, hydroxyalkylaminoalkyl, and heterocyclylalkyl, cycloalkyl, cycloalkenyl, cycloheteroalkyl, cycloheteroalkenyl, aryl, arylalkyl, and arylalkenyl, each of which is optionally substituted;
R₂, R₃, and R₄ represent four substituents each independently selected from the group consisting of hydrogen, halo, azido, alkoxy, cyano, nitro, hydroxy, amino, thio, and derivatives thereof; or any two adjacent substituents that are taken together with the attached carbons to form an optionally substituted heterocycle, and each of other two substituents is defined as above.

2. The method of claim 1, wherein the patient is selected for treatment with the compound of formula (I), or a pharmaceutically acceptable salt, hydrate, or solvate thereof, if the cancer cells in the sample are also MYC G4-positive, further comprising the step of determining if the cancer cells in the sample are MYC G4-positive.

3. The method of claim 1, wherein R₁ is a C₁-C₁₂ alkyl, alkenyl, heteroalkyl, heteroalkenyl, hydroxyalkyl, hydroxyalkylaminoalkyl, and heterocyclylalkyl, or heterocyclyl, each of which is optionally substituted.

4. The method of claim 3, wherein R₁ is selected from the group consisting of

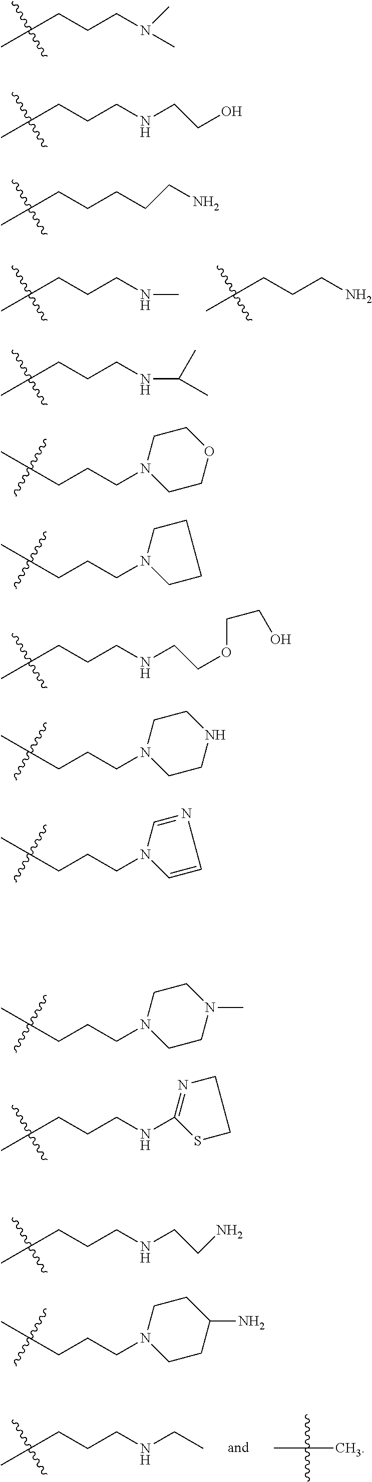

5. The method of claim 1, wherein D is N.

6. The method of claim 1, wherein E is N.

7. The method of claim 1, wherein the compound is selected from the group consisting of 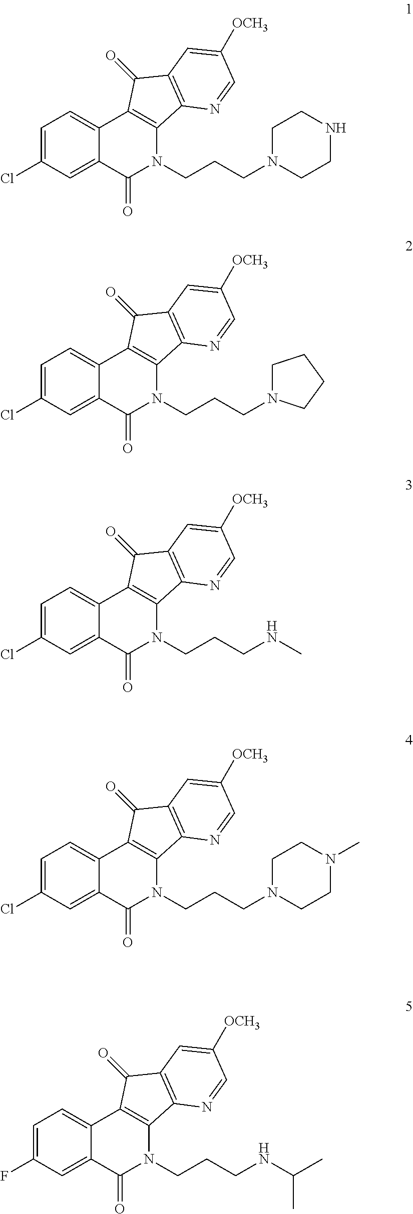 [SMILES: COc1cnc2c(c1)C(=O)c1c-2n(CCCN2CCNCC2)c(=O)c2cc(Cl)ccc12 ; COc1cnc2c(c1)C(=O)c1c-2n(CCCN2CCCC2)c(=O)c2cc(Cl)ccc12 ; CNCCCn1c2c(c3ccc(Cl)cc3c1=O)C(=O)c1cc(OC)cnc1-2 ; COc1cnc2c(c1)C(=O)c1c-2n(CCCN2CCN(C)CC2)c(=O)c2cc(Cl)ccc12 ; COc1cnc2c(c1)C(=O)c1c-2n(CCCNC(C)C)c(=O)c2cc(F)ccc12] 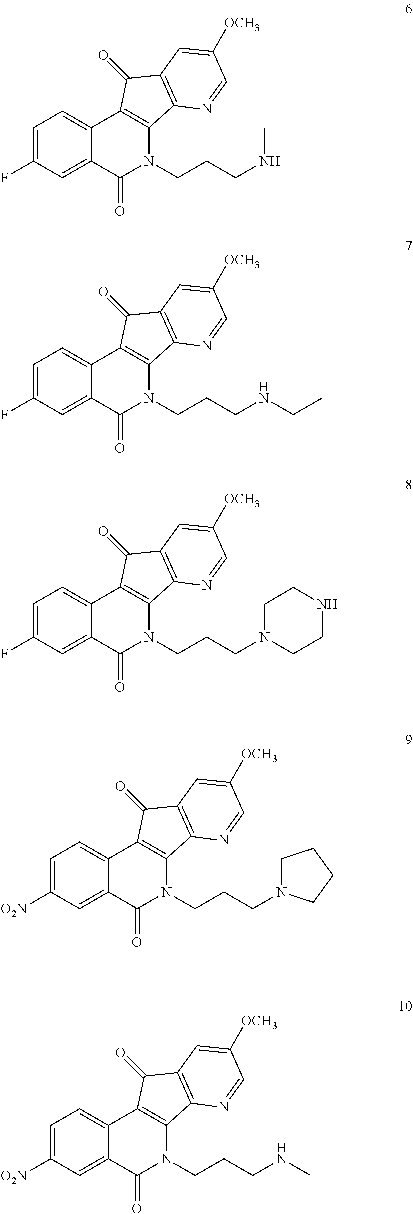 [SMILES: CNCCCn1c2c(c3ccc(F)cc3c1=O)C(=O)c1cc(OC)cnc1-2 ; CCNCCCn1c2c(c3ccc(F)cc3c1=O)C(=O)c1cc(OC)cnc1-2 ; COc1cnc2c(c1)C(=O)c1c-2n(CCCN2CCNCC2)c(=O)c2cc(F)ccc12 ; COc1cnc2c(c1)C(=O)c1c-2n(CCCN2CCCC2)c(=O)c2cc([N+](=O)[O-])ccc12 ; CNCCCn1c2c(c3ccc([N+](=O)[O-])cc3c1=O)C(=O)c1cc(OC)cnc1-2] 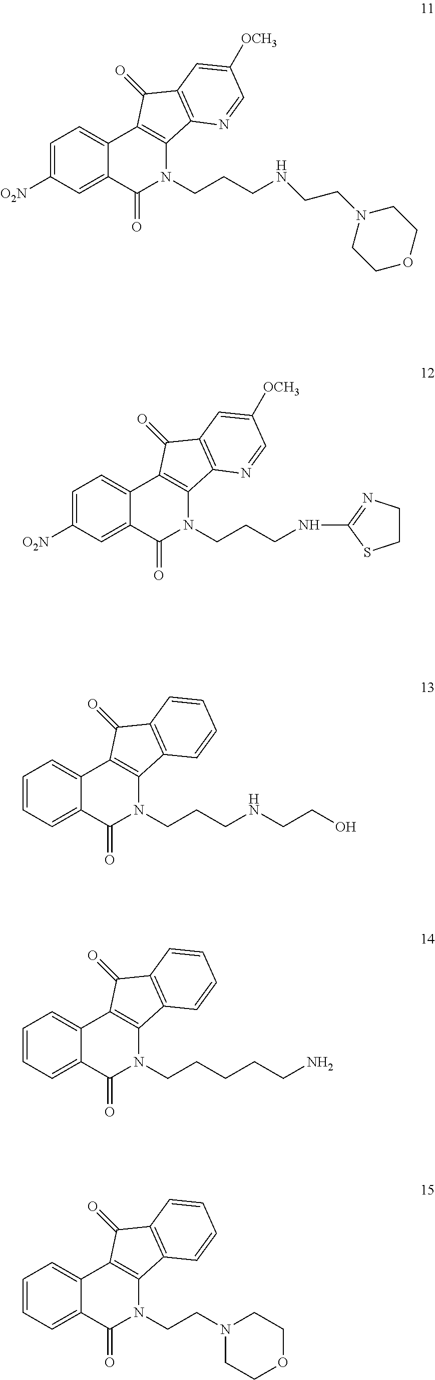 [SMILES: COc1cnc2c(c1)C(=O)c1c-2n(CCCNCCN2CCOCC2)c(=O)c2cc([N+](=O)[O-])ccc12 ; COc1cnc2c(c1)C(=O)c1c-2n(CCCNC2=NCCS2)c(=O)c2cc([N+](=O)[O-])ccc12 ; O=C1c2ccccc2-c2c1c1ccccc1c(=O)n2CCCNCCO ; NCCCCCn1c2c(c3ccccc3c1=O)C(=O)c1ccccc1-2 ; O=C1c2ccccc2-c2c1c1ccccc1c(=O)n2CCN1CCOCC1] 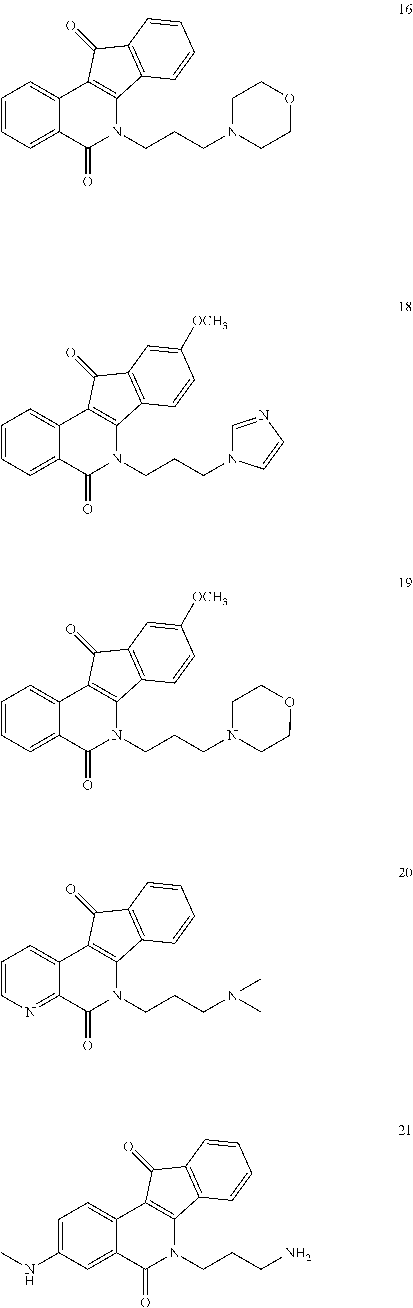 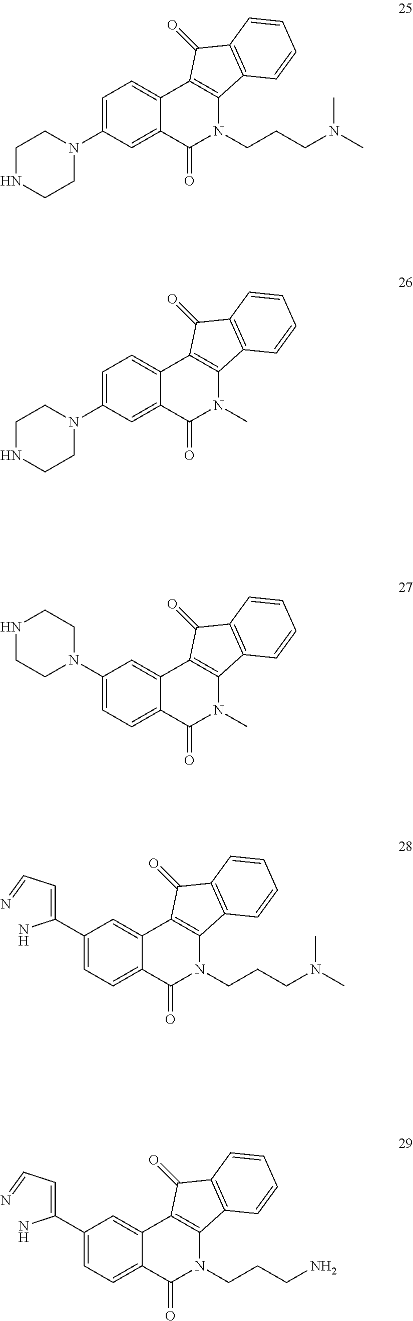 [SMILES: CN(C)CCCn1c2c(c3ccc(N4CCNCC4)cc3c1=O)C(=O)c1ccccc1-2 ; Cn1c2c(c3ccc(N4CCNCC4)cc3c1=O)C(=O)c1ccccc1-2 ; Cn1c2c(c3cc(N4CCNCC4)ccc3c1=O)C(=O)c1ccccc1-2 ; CN(C)CCCn1c2c(c3cc(/C(N)=C/C=N)ccc3c1=O)C(=O)c1ccccc1-2 ; N=C/C=C(\N)c1ccc2c(=O)n(CCCN)c3c(c2c1)C(=O)c1ccccc1-3] 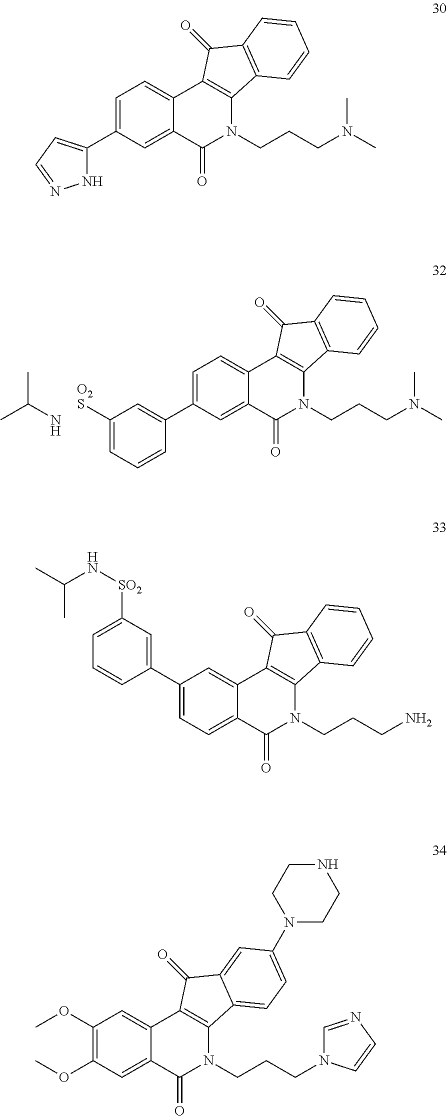 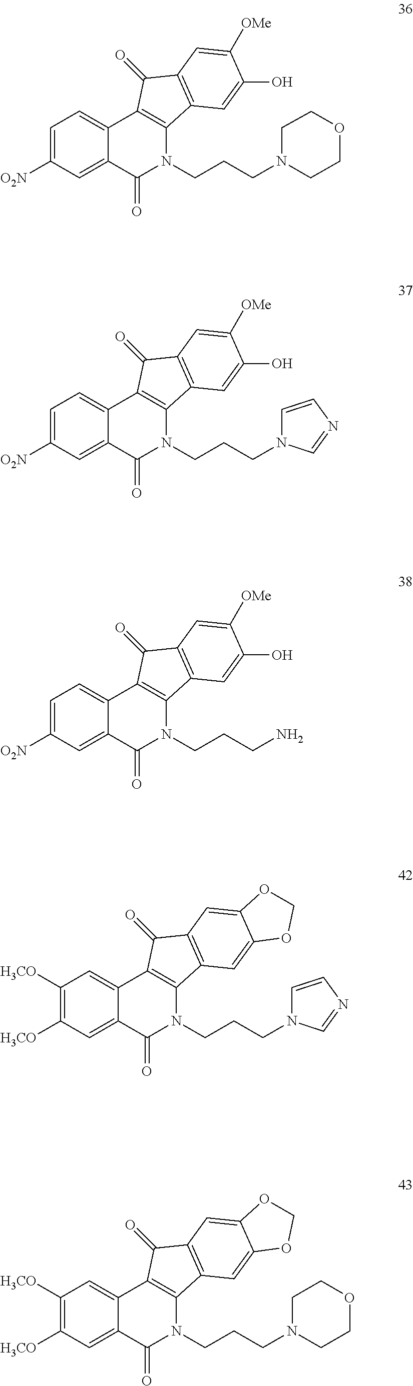 [SMILES: COc1cc2c(cc1O)-c1c(c3ccc([N+](=O)[O-])cc3c(=O)n1CCCN1CCOCC1)C2=O ; COc1cc2c(cc1O)-c1c(c3ccc([N+](=O)[O-])cc3c(=O)n1CCCn1ccnc1)C2=O ; COc1cc2c(cc1O)-c1c(c3ccc([N+](=O)[O-])cc3c(=O)n1CCCN)C2=O ; COc1cc2c3c(n(CCCn4ccnc4)c(=O)c2cc1OC)-c1cc2c(cc1C3=O)OCO2 ; COc1cc2c3c(n(CCCN4CCOCC4)c(=O)c2cc1OC)-c1cc2c(cc1C3=O)OCO2] 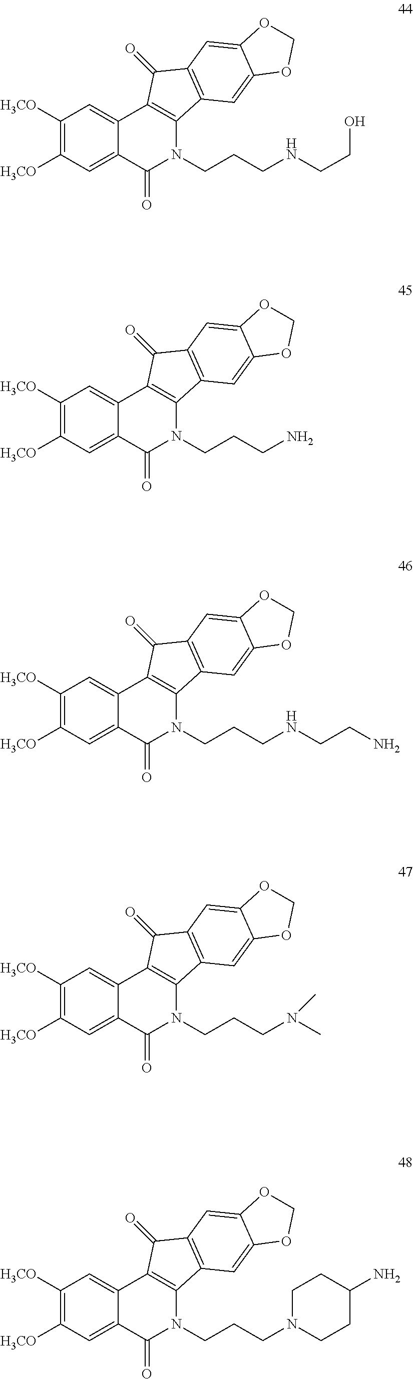 [SMILES: COc1cc2c3c(n(CCCNCCO)c(=O)c2cc1OC)-c1cc2c(cc1C3=O)OCO2 ; COc1cc2c3c(n(CCCN)c(=O)c2cc1OC)-c1cc2c(cc1C3=O)OCO2 ; COc1cc2c3c(n(CCCNCCN)c(=O)c2cc1OC)-c1cc2c(cc1C3=O)OCO2 ; COc1cc2c3c(n(CCCN(C)C)c(=O)c2cc1OC)-c1cc2c(cc1C3=O)OCO2 ; COc1cc2c3c(n(CCCN4CCC(N)CC4)c(=O)c2cc1OC)-c1cc2c(cc1C3=O)OCO2] 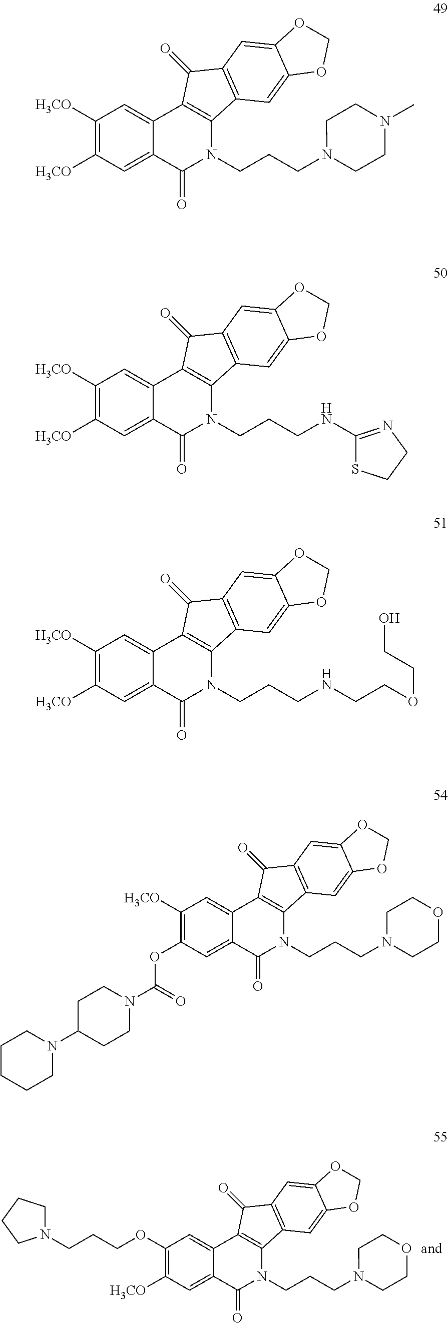 [SMILES: COc1cc2c3c(n(CCCN4CCN(C)CC4)c(=O)c2cc1OC)-c1cc2c(cc1C3=O)OCO2 ; COc1cc2c3c(n(CCCNC4=NCCS4)c(=O)c2cc1OC)-c1cc2c(cc1C3=O)OCO2 ; COc1cc2c3c(n(CCCNCCOCCO)c(=O)c2cc1OC)-c1cc2c(cc1C3=O)OCO2 ; COc1cc2c3c(n(CCCN4CCOCC4)c(=O)c2cc1OC(=O)N1CCC(N2CCCCC2)CC1)-c1cc2c(cc1C3=O)OCO2 ; COc1cc2c(=O)n(CCCN3CCOCC3)c3c(c2cc1OCCCN1CCCC1)C(=O)c1cc2c(cc1-3)OCO2] 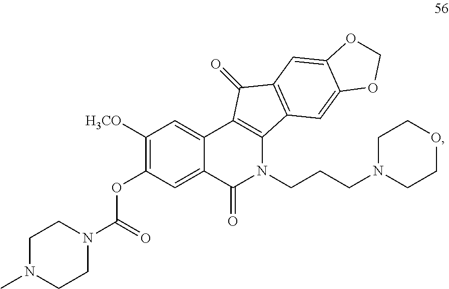 [SMILES: COc1cc2c3c(n(CCCN4CCOCC4)c(=O)c2cc1OC(=O)N1CCN(C)CC1)-c1cc2c(cc1C3=O)OCO2]
or a pharmaceutically acceptable salt, hydrate, or solvate thereof.

8. The method of claim 7, wherein the compound is selected from the group consisting 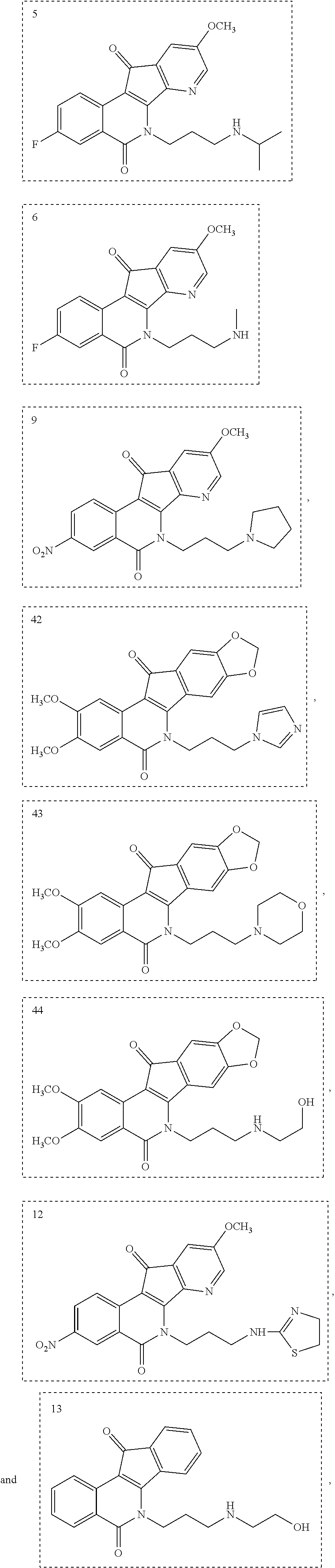
or a pharmaceutically acceptable salt, hydrate, or solvate thereof.

9. The method of claim 1, wherein the compound is a human topoisomerase I inhibitor.

10. The method of claim 1, wherein the compound is selected from 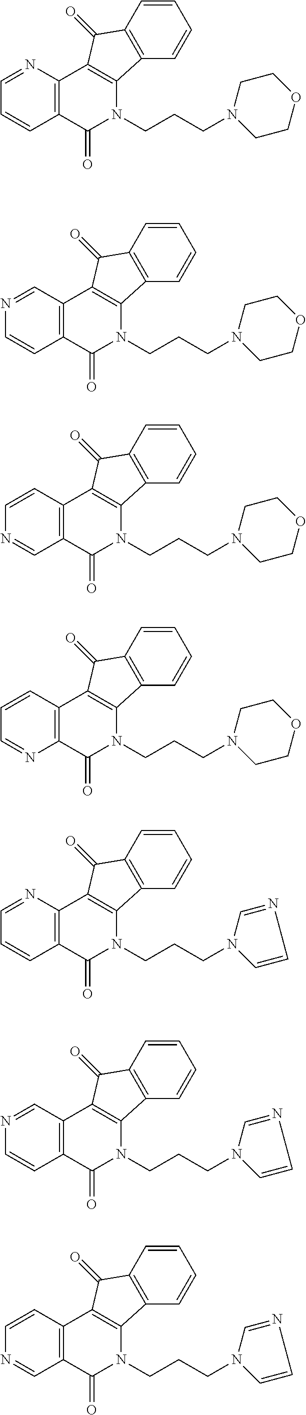 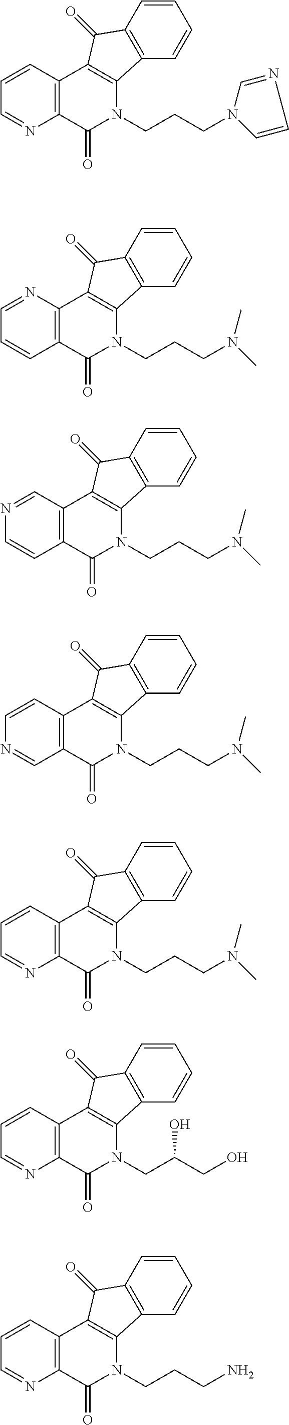 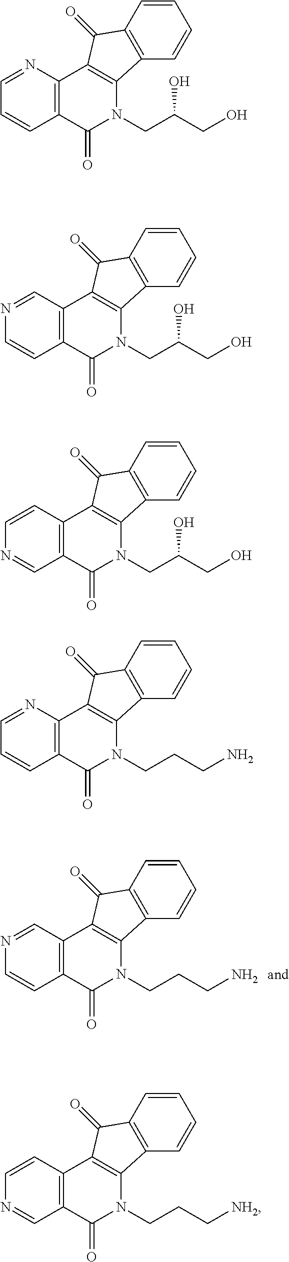 [SMILES: O=C1c2ccccc2-c2c1c1ncccc1c(=O)n2C[C@H](O)CO ; O=C1c2ccccc2-c2c1c1cnccc1c(=O)n2C[C@H](O)CO ; O=C1c2ccccc2-c2c1c1ccncc1c(=O)n2C[C@H](O)CO ; NCCCn1c2c(c3ncccc3c1=O)C(=O)c1ccccc1-2 ; NCCCn1c2c(c3cnccc3c1=O)C(=O)c1ccccc1-2 ; NCCCn1c2c(c3ccncc3c1=O)C(=O)c1ccccc1-2]
or a pharmaceutically acceptable salt, hydrate, or solvate thereof.

11. The method of claim 1 wherein the compound is a compound of formula (II): 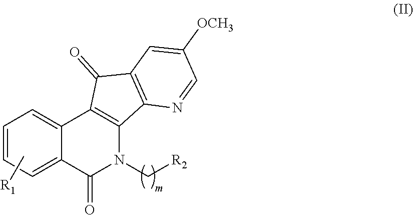
or a pharmaceutically acceptable salt, hydrate, or solvate thereof, wherein m is 3, R¹ is 3-Cl or 3-F, and R² is selected from the group consisting of

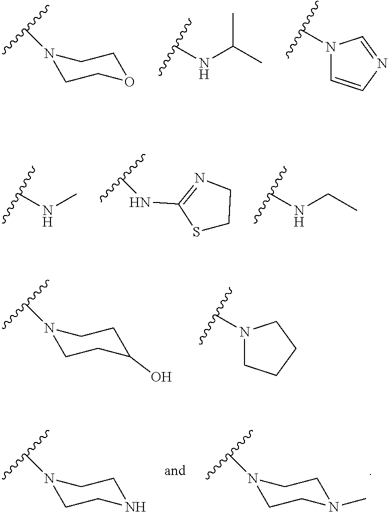

12. The method of claim 11 wherein R¹ is 3-F.

13. The method of claim 11 wherein R¹ is 3-Cl.

14. The method of claim 11 wherein R¹ is 3-NO₂.

15. The method of claim 12 wherein R² is MeHN—, EtHN—, or i-PrHN—.

16. The method of claim 13 wherein R² is MeHN—, EtHN—, or i-PrHN—.

17. The method of claim 14 wherein R² is MeHN—, EtHN—, or i-PrHN—.
